SEQUENCE LISTING (48 sequences)

SEQ ID NO: 1 - DNA, 5444 bp, Homo sapiens
ctacctccaaccatgggccttttgggaatactttgttttttaatcttcctggggaaaacctggggacaggagcaaacatatgtcatttcagcaccaaaaatattccgtgttggagcatctgaaaatattgtgattcaagtttatggatacactgaagcatttgatgcaacaatctctattaaaagttatcctgataaaaaatttagttactcctcaggccatgttcatttatcctcagagaataaattccaaaactctgcaatcttaacaatacaaccaaaacaattgcctggaggacaaaacccagtttcttatgtgtatttggaagttgtatcaaagcatttttcaaaatcaaaaagaatgccaataacctatgacaatggatttctcttcattcatacagacaaacctgtttatactccagaccagtcagtaaaagttagagtttattcgttgaatgacgacttgaagccagccaaaagagaaactgtcttaaccttcatagatcctgaaggatcagaagttgacatggtagaagaaattgatcatattggaattatctcttttcctgacttcaagattccgtctaatcctagatatggtatgtggacgatcaaggctaaatataaagaggacttttcaacaactggaaccgcatattttgaagttaaagaatatgtcttgccacatttttctgtctcaatcgagccagaatataatttcattggttacaagaactttaagaattttgaaattactataaaagcaagatatttttataataaagtagtcactgaggctgacgtttatatcacatttggaataagagaagacttaaaagatgatcaaaaagaaatgatgcaaacagcaatgcaaaacacaatgttgataaatggaattgctcaagtcacatttgattctgaaacagcagtcaaagaactgtcatactacagtttagaagatttaaacaacaagtacctttatattgctgtaacagtcatagagtctacaggtggattttctgaagaggcagaaatacctggcatcaaatatgtcctctctccctacaaactgaatttggttgctactcctcttttcctgaagcctgggattccatatcccatcaaggtgcaggttaaagattcgcttgaccagttggtaggaggagtcccagtaatactgaatgcacaaacaattgatgtaaaccaagagacatctgacttggatccaagcaaaagtgtaacacgtgttgatgatggagtagcttcctttgtgcttaatctcccatctggagtgacggtgctggagtttaatgtcaaaactgatgctccagatcttccagaagaaaatcaggccagggaaggttaccgagcaatagcatactcatctctcagccaaagttacctttatattgattggactgataaccataaggctttgctagtgggagaacatctgaatattattgttacccccaaaagcccatatattgacaaaataactcactataattacttgattttatccaagggcaaaattatccattttggcacgagggagaaattttcagatgcatcttatcaaagtataaacattccagtaacacagaacatggttccttcatcccgacttctggtctattatatcgtcacaggagaacagacagcagaattagtgtctgattcagtctggttaaatattgaagaaaaatgtggcaaccagctccaggttcatctgtctcctgatgcagatgcatattctccaggccaaactgtgtctcttaatatggcaactggaatggattcctgggtggcattagcagcagtggacagtgctgtgtatggagtccaaagaggagccaaaaagcccttggaaagagtatttcaattcttagagaagagtgatctgggctgtggggcaggtggtggcctcaacaatgccaatgtgttccacctagctggacttaccttcctcactaatgcaaatgcagatgactcccaagaaaatgatgaaccttgtaaagaaattctcaggccaagaagaacgctgcaaaagaagatagaagaaatagctgctaaatataaacattcagtagtgaagaaatgttgttacgatggagcctgcgttaataatgatgaaacctgtgagcagcgagctgcacggattagtttagggccaagatgcatcaaagctttcactgaatgttgtgtcgtcgcaagccagctccgtgctaatatctctcataaagacatgcaattgggaaggctacacatgaagaccctgttaccagtaagcaagccagaaattcggagttattttccagaaagctggttgtgggaagttcatcttgttcccagaagaaaacagttgcagtttgccctacctgattctctaaccacctgggaaattcaaggcattggcatttcaaacactggtatatgtgttgctgatactgtcaaggcaaaggtgttcaaagatgtcttcctggaaatgaatataccatattctgttgtacgaggagaacagatccaattgaaaggaactgtttacaactataggacttctgggatgcagttctgtgttaaaatgtctgctgtggagggaatctgcacttcggaaagcccagtcattgatcatcagggcacaaagtcctccaaatgtgtgcgccagaaagtagagggctcctccagtcacttggtgacattcactgtgcttcctctggaaattggccttcacaacatcaatttttcactggagacttggtttggaaaagaaatcttagtaaaaacattacgagtggtgccagaaggtgtcaaaagggaaagctattctggtgttactttggatcctaggggtatttatggtaccattagcagacgaaaggagttcccatacaggatacccttagatttggtccccaaaacagaaatcaaaaggattttgagtgtaaaaggactgcttgtaggtgagatcttgtctgcagttctaagtcaggaaggcatcaatatcctaacccacctccccaaagggagtgcagaggcggagctgatgagcgttgtcccagtattctatgtttttcactacctggaaacaggaaatcattggaacatttttcattctgacccattaattgaaaagcagaaactgaagaaaaaattaaaagaagggatgttgagcattatgtcctacagaaatgctgactactcttacagtgtgtggaagggtggaagtgctagcacttggttaacagcttttgctttaagagtacttggacaagtaaataaatacgtagagcagaaccaaaattcaatttgtaattctttattgtggctagttgagaattatcaattagataatggatctttcaaggaaaattcacagtatcaaccaataaaattacagggtaccttgcctgttgaagcccgagagaacagcttatatcttacagcctttactgtgattggaattagaaaggctttcgatatatgccccctggtgaaaatcgacacagctctaattaaagctgacaactttctgcttgaaaatacactgccagcccagagcacctttacattggccatttctgcgtatgctctttccctgggagataaaactcacccacagtttcgttcaattgtttcagctttgaagagagaagctttggttaaaggtaatccacccatttatcgtttttggaaagacaatcttcagcataaagacagctctgtacctaacactggtacggcacgtatggtagaaacaactgcctatgctttactcaccagtctgaacttgaaagatataaattatgttaacccagtcatcaaatggctatcagaagagcagaggtatggaggtggcttttattcaacccaggacaccatcaatgccattgagggcctgacggaatattcactcctggttaaacaactccgcttgagtatggacatcgatgtttcttacaagcataaaggtgccttacataattataaaatgacagacaagaatttccttgggaggccagtagaggtgcttctcaatgatgacctcattgtcagtacaggatttggcagtggcttggctacagtacatgtaacaactgtagttcacaaaaccagtacctctgaggaagtttgcagcttttatttgaaaatcgatactcaggatattgaagcatcccactacagaggctacggaaactctgattacaaacgcatagtagcatgtgccagctacaagcccagcagggaagaatcatcatctggatcctctcatgcggtgatggacatctccttgcctactggaatcagtgcaaatgaagaagacttaaaagcccttgtggaaggggtggatcaactattcactgattaccaaatcaaagatggacatgttattctgcaactgaattcgattccctccagtgatttcctttgtgtacgattccggatatttgaactctttgaagttgggtttctcagtcctgccactttcacagtttacgaataccacagaccagataaacagtgtaccatgttttatagcacttccaatatcaaaattcagaaagtctgtgaaggagccgcgtgcaagtgtgtagaagctgattgtgggcaaatgcaggaagaattggatctgacaatctctgcagagacaagaaaacaaacagcatgtaaaccagagattgcatatgcttataaagttagcatcacatccatcactgtagaaaatgtttttgtcaagtacaaggcaacccttctggatatctacaaaactggggaagctgttgctgagaaagactctgagattaccttcattaaaaaggtaacctgtactaacgctgagctggtaaaaggaagacagtacttaattatgggtaaagaagccctccagataaaatacaatttcagtttcaggtacatctaccctttagattccttgacctggattgaatactggcctagagacacaacatgttcatcgtgtcaagcatttttagctaatttagatgaatttgccgaagatatctttttaaatggatgctaaaattcctgaagttcagctgcatacagtttgcacttatggactcctgttgttgaagttcgtttttttgttttcttctttttttaaacattcatagctggtcttatttgtaaagctcactttacttagaattagtggcacttgcttttattagagaatgatttcaaatgctgtaactttctgaaataacatggccttggagggcatgaagacagatactcctccaaggttattggacaccggaaacaataaattggaacacctcctcaaacctaccactcaggaatgtttgctggggccgaaagaacagtccattgaaagggagtattacaaaaacatggcctttgcttgaaagaaaataccaaggaacaggaaactgatcattaaagcctgagtttgctttc

SEQ ID NO: 2 - protein, 1676 aa, Homo sapiens
MGLLGILCFLIFLGKTWGQEQTYVISAPKIFRVGASENIVIQVYGYTEAFDATISIKSYPDKKFSYSSGHVHLSSENKFQNSAILTIQPKQLPGGQNPVSYVYLEVVSKHFSKSKRMPITYDNGFLFIHTDKPVYTPDQSVKVRVYSLNDDLKPAKRETVLTFIDPEGSEVDMVEEIDHIGIISFPDFKIPSNPRYGMWTIKAKYKEDFSTTGTAYFEVKEYVLPHFSVSIEPEYNFIGYKNFKNFEITIKARYFYNKVVTEADVYITFGIREDLKDDQKEMMQTAMQNTMLINGIAQVTFDSETAVKELSYYSLEDLNNKYLYIAVTVIESTGGFSEEAEIPGIKYVLSPYKLNLVATPLFLKPGIPYPIKVQVKDSLDQLVGGVPVILNAQTIDVNQETSDLDPSKSVTRVDDGVASFVLNLPSGVTVLEFNVKTDAPDLPEENQAREGYRAIAYSSLSQSYLYIDWTDNHKALLVGEHLNIIVTPKSPYIDKITHYNYLILSKGKIIHFGTREKFSDASYQSINIPVTQNMVPSSRLLVYYIVTGEQTAELVSDSVWLNIEEKCGNQLQVHLSPDADAYSPGQTVSLNMATGMDSWVALAAVDSAVYGVQRGAKKPLERVFQFLEKSDLGCGAGGGLNNANVFHLAGLTFLTNANADDSQENDEPCKEILRPRRTLQKKIEEIAAKYKHSVVKKCCYDGACVNNDETCEQRAARISLGPRCIKAFTECCVVASQLRANISHKDMQLGRLHMKTLLPVSKPEIRSYFPESWLWEVHLVPRRKQLQFALPDSLTTWEIQGIGISNTGICVADTVKAKVFKDVFLEMNIPYSVVRGEQIQLKGTVYNYRTSGMQFCVKMSAVEGICTSESPVIDHQGTKSSKCVRQKVEGSSSHLVTFTVLPLEIGLHNINFSLETWFGKEILVKTLRVVPEGVKRESYSGVTLDPRGIYGTISRRKEFPYRIPLDLVPKTEIKRILSVKGLLVGEILSAVLSQEGINILTHLPKGSAEAELMSVVPVFYVFHYLETGNHWNIFHSDPLIEKQKLKKKLKEGMLSIMSYRNADYSYSVWKGGSASTWLTAFALRVLGQVNKYVEQNQNSICNSLLWLVENYQLDNGSFKENSQYQPIKLQGTLPVEARENSLYLTAFTVIGIRKAFDICPLVKIDTALIKADNFLLENTLPAQSTFTLAISAYALSLGDKTHPQFRSIVSALKREALVKGNPPIYRFWKDNLQHKDSSVPNTGTARMVETTAYALLTSLNLKDINYVNPVIKWLSEEQRYGGGFYSTQDTINAIEGLTEYSLLVKQLRLSMDIDVSYKHKGALHNYKMTDKNFLGRPVEVLLNDDLIVSTGFGSGLATVHVTTVVHKTSTSEEVCSFYLKIDTQDIEASHYRGYGNSDYKRIVACASYKPSREESSSGSSHAVMDISLPTGISANEEDLKALVEGVDQLFTDYQIKDGHVILQLNSIPSSDFLCVRFRIFELFEVGFLSPATFTVYEYHRPDKQCTMFYSTSNIKIQKVCEGAACKCVEADCGQMQEELDLTISAETRKQTACKPEIAYAYKVSITSITVENVFVKYKATLLDIYKTGEAVAEKDSEITFIKKVTCTNAELVKGRQYLIMGKEALQIKYNFSFRYIYPLDSLTWIEYWPRDTTCSSCQAFLANLDEFAEDIFLNGC

SEQ ID NO: 3 - protein, 1658 aa, Homo sapiens
QEQTYVISAPKIFRVGASENIVIQVYGYTEAFDATISIKSYPDKKFSYSSGHVHLSSENKFQNSAILTIQPKQLPGGQNPVSYVYLEVVSKHFSKSKRMPITYDNGFLFIHTDKPVYTPDQSVKVRVYSLNDDLKPAKRETVLTFIDPEGSEVDMVEEIDHIGIISFPDFKIPSNPRYGMWTIKAKYKEDFSTTGTAYFEVKEYVLPHFSVSIEPEYNFIGYKNFKNFEITIKARYFYNKVVTEADVYITFGIREDLKDDQKEMMQTAMQNTMLINGIAQVTFDSETAVKELSYYSLEDLNNKYLYIAVTVIESTGGFSEEAEIPGIKYVLSPYKLNLVATPLFLKPGIPYPIKVQVKDSLDQLVGGVPVILNAQTIDVNQETSDLDPSKSVTRVDDGVASFVLNLPSGVTVLEFNVKTDAPDLPEENQAREGYRAIAYSSLSQSYLYIDWTDNHKALLVGEHLNIIVTPKSPYIDKITHYNYLILSKGKIIHFGTREKFSDASYQSINIPVTQNMVPSSRLLVYYIVTGEQTAELVSDSVWLNIEEKCGNQLQVHLSPDADAYSPGQTVSLNMATGMDSWVALAAVDSAVYGVQRGAKKPLERVFQFLEKSDLGCGAGGGLNNANVFHLAGLTFLTNANADDSQENDEPCKEILRPRRTLQKKIEEIAAKYKHSVVKKCCYDGACVNNDETCEQRAARISLGPRCIKAFTECCVVASQLRANISHKDMQLGRLHMKTLLPVSKPEIRSYFPESWLWEVHLVPRRKQLQFALPDSLTTWEIQGIGISNTGICVADTVKAKVFKDVFLEMNIPYSVVRGEQIQLKGTVYNYRTSGMQFCVKMSAVEGICTSESPVIDHQGTKSSKCVRQKVEGSSSHLVTFTVLPLEIGLHNINFSLETWFGKEILVKTLRVVPEGVKRESYSGVTLDPRGIYGTISRRKEFPYRIPLDLVPKTEIKRILSVKGLLVGEILSAVLSQEGINILTHLPKGSAEAELMSVVPVFYVFHYLETGNHWNIFHSDPLIEKQKLKKKLKEGMLSIMSYRNADYSYSVWKGGSASTWLTAFALRVLGQVNKYVEQNQNSICNSLLWLVENYQLDNGSFKENSQYQPIKLQGTLPVEARENSLYLTAFTVIGIRKAFDICPLVKIDTALIKADNFLLENTLPAQSTFTLAISAYALSLGDKTHPQFRSIVSALKREALVKGNPPIYRFWKDNLQHKDSSVPNTGTARMVETTAYALLTSLNLKDINYVNPVIKWLSEEQRYGGGFYSTQDTINAIEGLTEYSLLVKQLRLSMDIDVSYKHKGALHNYKMTDKNFLGRPVEVLLNDDLIVSTGFGSGLATVHVTTVVHKTSTSEEVCSFYLKIDTQDIEASHYRGYGNSDYKRIVACASYKPSREESSSGSSHAVMDISLPTGISANEEDLKALVEGVDQLFTDYQIKDGHVILQLNSIPSSDFLCVRFRIFELFEVGFLSPATFTVYEYHRPDKQCTMFYSTSNIKIQKVCEGAACKCVEADCGQMQEELDLTISAETRKQTACKPEIAYAYKVSITSITVENVFVKYKATLLDIYKTGEAVAEKDSEITFIKKVTCTNAELVKGRQYLIMGKEALQIKYNFSFRYIYPLDSLTWIEYWPRDTTCSSCQAFLANLDEFAEDIFLNGC

SEQ ID NO: 4 - protein, 21 aa, Homo sapiens
VIDHQGTKSSKCVRQKVEGSS

SEQ ID NO: 5 - DNA, 95 bp, Homo sapiens
tatatccgtggtttcctgctacctccaaccatgggccttttgggaatactttgttttttaatcttcctggggaaaacctggggacaggagcaaac

SEQ ID NO: 6 - DNA, 193 bp, Homo sapiens
atatgtcatttcagcaccaaaaatattccgtgttggagcatctgaaaatattgtgattcaagtttatggatacactgaagcatttgatgcaacaatctctattaaaagttatcctgataaaaaatttagttactcctcaggccatgttcatttatcctcagagaataaattccaaaactctgcaatcttaaca

SEQ ID NO: 7 - DNA, 163 bp, Homo sapiens
atacaaccaaaacaattgcctggaggacaaaacccagtttcttatgtgtatttggaagttgtatcaaagcatttttcaaaatcaaaaagaatgccaataacctatgacaatggatttctcttcattcatacagacaaacctgtttatactccagaccagtcag

SEQ ID NO: 8 - DNA, 71 bp, Homo sapiens
taaaagttagagtttattcgttgaatgacgacttgaagccagccaaaagagaaactgtcttaactttcata

SEQ ID NO: 9 - DNA, 92 bp, Homo sapiens
gatcctgaaggatcagaagttgacatggtagaagaaattgatcatattggaattatctcttttcctgacttcaagattccgtctaatcctag

SEQ ID NO: 10 - DNA, 83 bp, Homo sapiens
atatggtatgtggacgatcaaggctaaatataaagaggacttttcaacaactggaaccgcatattttgaagttaaagaatatg

SEQ ID NO: 11 - DNA, 91 bp, Homo sapiens
aagtcaaaacctcatttcaattatttgctaacccaaacacaaaattaagtttatggggaagctttacgagaactgtcaagtttgtttaacc

SEQ ID NO: 12 - DNA, 115 bp, Homo sapiens
atatttttataataaagtagtcactgaggctgacgtttatatcacatttggaataagagaagacttaaaagatgatcaaaaagaaatgatgcaaacagcaatgcaaaacacaatg

SEQ ID NO: 13 - DNA, 127 bp, Homo sapiens
ttgataaatggaattgctcaagtcacatttgattctgaaacagcagtcaaagaactgtcatactacagtttagaagatttaaacaacaagtacctttatattgctgtaacagtcatagagtctacag

SEQ ID NO: 14 - DNA, 116 bp, Homo sapiens
gtggattttctgaagaggcagaaatacctggcatcaaatatgtcctctctccctacaaactgaatttggttgctactcctcttttcctgaagcctgggattccatatcccatcaag

SEQ ID NO: 15 - DNA, 186 bp, Homo sapiens
gtgcaggttaaagattcgcttgaccagttggtaggaggagtcccagtaacactgaatgcacaaacaattgatgtaaaccaagagacatctgacttggatccaagcaaaagtgtaacacgtgttgatgatggagtagcttcctttgtgcttaatctcccatctggagtgacggtgctggagtttaat

SEQ ID NO: 16 - DNA, 204 bp, Homo sapiens
gtcaaaactgatgctccagatcttccagaagaaaatcaggccagggaaggttaccgagcaatagcatactcatctctcagccaaagttacctttatattgattggactgataaccataaggctttgctagtgggagaacatctgaatattattgttacccccaaaagcccatatattgacaaaataactcactataattacttg

SEQ ID NO: 17 - DNA, 210 bp, Homo sapiens
attttatccaagggcaaaattatccactttggcacgagggagaaattttcagatgcatcttatcaaagtataaacattccagtaacacagaacatggttccttcatcccgacttctggtctattacatcgtcacaggagaacagacagcagaattagtgtctgattcagtctggttaaatattgaagaaaaatgtggcaaccagctccag

SEQ ID NO: 18 - DNA, 150 bp, Homo sapiens
catgcttgtaggttcttacatatatatactatatatagaaaataatatataacaaagtatcatataatgcatatacagtaatatataatatatatactaaaatatgtaaatatagcaatattatatagaacataagcttaaaatatgttt

SEQ ID NO: 19 - DNA, 130 bp, Homo sapiens
gtatttcaattcttagagaagagtgatctgggctgtggggcaggtggtggcctcaacaatgccaatgtgttccacctagctggacttaccttcctcactaatgcaaatgcagatgactcccaagaaaatg

SEQ ID NO: 20 - DNA, 63 bp, Homo sapiens
atgaaccttgtaaagaaattctcaggccaagaagaacgctgcaaaagaagatagaagaaatag

SEQ ID NO: 21 - DNA, 198 bp, Homo sapiens
ctgctaaatataaacattcagtagtgaagaaatgttgttacgatggagcctgcgttaataatgatgaaacctgtgagcagcgagctgcacggattagtttagggccaagatgcatcaaagctttcactgaatgttgtgtcgtcgcaagccagctccgtgctaatatctctcataaagacatgcaattgggaaggctac

SEQ ID NO: 22 - DNA, 91 bp, Homo sapiens
acatgaagaccctgttaccagtaagcaagccagaaattcggagttattttccagaaagctggttgtgggaagttcatcttgttcccagaag

SEQ ID NO: 23 - DNA, 74 bp, Homo sapiens
aaaacagttgcagtttgccctacctgattctctaaccacctgggaaattcaaggcgttggcatttcaaacactg

SEQ ID NO: 24 - DNA, 140 bp, Homo sapiens
gtatatgtgttgctgatactgtcaaggcaaaggtgttcaaagatgtcttcctggaaatgaatataccatattctgttgtacgaggagaacagatccaattgaaaggaactgtttacaactataggacttctgggatgcag

SEQ ID NO: 25 - DNA, 228 bp, Homo sapiens
ttctgtgttaaaatgtctgctgtggagggaatctgcacttcggaaagcccagtcattgatcatcagggcacaaagtcctccaaatgtgtgcgccagaaagtagagggctcctccagtcacttggtgacattcactgtgcttcctctggaaattggccttcacaacatcaatttttcactggagacttggtttggaaaagaaatcttagtaaaaacattacgagtggtg

SEQ ID NO: 26 - DNA, 61 bp, Homo sapiens
ccagaaggtgtcaaaagggaaagctattctggtgttactttggatcctaggggtatttatg

SEQ ID NO: 27 - DNA, 90 bp, Homo sapiens
gtaccattagcagacgaaaggagttcccatacaggatacccttagatttggtccccaaaacagaaatcaaaaggattttgagtgtaaaag

SEQ ID NO: 28 - DNA, 213 bp, Homo sapiens
gactgcttgtaggtgagatcttgtctgcagttctaagtcaggaaggcatcaatatcctaacccacctccccaaagggagtgcagaggcggagctgatgagcgttgtcccagtattctatgtttttcactacctggaaacaggaaatcattggaacatttttcattctgacccattaattgaaaagcagaaactgaagaaaaaattaaaagaag

SEQ ID NO: 29 - DNA, 76 bp, Homo sapiens
ggatgttgagcattatgtcctacagaaatgctgactactcttacagtgtgtggaagggtggaagtgctagcacttg

SEQ ID NO: 30 - DNA, 160 bp, Homo sapiens
gttaacagcttttgctttaagagtacttggacaagtaaataaatacgtagagcagaaccaaaattcaatttgtaattctttattgtggctagttgagaattatcaattagataatggatctttcaaggaaaattcacagtatcaaccaataaaattacag

SEQ ID NO: 31 - DNA, 96 bp, Homo sapiens
ggtaccttgcctgttgaagcccgagagaacagcttatatcttacagcctttactgtgattggaattagaaaggctttcgatatatgccccctggtg

SEQ ID NO: 32 - DNA, 172 bp, Homo sapiens
aaaatcgacacagctctaattaaagctgacaactttctgcttgaaaatacactgccagcccagagcacctttacattggccatttctgcgtatgctctttccctgggagataaaactcacccacagtttcgttcaattgtttcagctttgaagagagaagctttggttaaag

SEQ ID NO: 33 - DNA, 206 bp, Homo sapiens
gtaatccacccatttatcgtttttggaaagacaatcttcagcataaagacagctctgtacctaacactggtacggcacgtatggtagaaacaactgcctatgctttactcaccagtctgaacttgaaagatataaattatgttaacccagtcatcaaatggctatcagaagagcagaggtatggaggtggcttttattcaacccag

SEQ ID NO: 34 - DNA, 153 bp, Homo sapiens
gacacaatcaatgccattgagggcctgacggaatattcactcctggttaaacaactccgcttgagtatggacatcgatgtttcttacaagcataaaggtgccttacataattataaaatgacagacaagaatttccttgggaggccagtagag

SEQ ID NO: 35 - DNA, 63 bp, Homo sapiens
gtgcttctcaatgatgacctcattgtcagtacaggatttggcagtggcttggctacagtacat

SEQ ID NO: 36 - DNA, 82 bp, Homo sapiens
gtaacaactgtagttcacaaaaccagtacctctgaggaagtttgcagcttttatttgaaaatcgatactcaggatattgaag

SEQ ID NO: 37 - DNA, 58 bp, Homo sapiens
catcccactacagaggctacggaaactctgattacaaacgcatagtagcatgtgccag

SEQ ID NO: 38 - DNA, 103 bp, Homo sapiens
ctacaagcccagcagggaagaatcatcatctggatcctctcatgcggtgatggacatctccttgcctactggaatcagtgcaaatgaagaagacttaaaagcc

SEQ ID NO: 39 - DNA, 75 bp, Homo sapiens
cttgtggaaggggtggatcaactattcactgattaccaaatcaaagatggacatgttattctgcaactgaattcg

SEQ ID NO: 40 - DNA, 106 bp, Homo sapiens
attccctccagtgatttcctttgtgtacgattccggatatttgaactctttgaagttgggtttctcagtcctgccactttcacagtgtacgaataccacagaccag

SEQ ID NO: 41 - DNA, 84 bp, Homo sapiens
ataaacagtgtaccatgttttatagcacttccaatatcaaaattcagaaagtctgtgaaggagccgcgtgcaagtgtgtagaag

SEQ ID NO: 42 - DNA, 90 bp, Homo sapiens
ctgattgtgggcaaatgcaggaagaattggatctgacaatctctgcagagacaagaaaacaaacagcatgtaaaccagagattgcatatg

SEQ ID NO: 43 - DNA, 84 bp, Homo sapiens
cttataaagttagcatcacatccatcactgtagaaaatgtttttgtcaagtacaaggcaacccttctggatatctacaaaactg

SEQ ID NO: 44 - DNA, 139 bp, Homo sapiens
gggaagctgttgctgagaaagactctgagattaccttcattaaaaaggtaacctgtactaacgctgagctggtaaaaggaagacagtacttaattatgggtaaagaagccctccagataaaatacaatttcagtttcag

SEQ ID NO: 45 - DNA, 534 bp, Homo sapiens
gtacatctaccctttagattccttgacctggattgaatactggcctagagacacaacatgttcatcgtgtcaagcatttttagctaatttagatgaatttgccgaagatatctttttaaatggatgctaaaattcctgaagttcagctgcatacagtttgcacttatggactcctgttgttgaagttcgtttttttgttttcttctttttttaaacattcatagctggtcttatttgtaaagctcactttacttagaattagtggcacttgcttttattagagaatgatttcaaatgctgtaactttctgaaataacatggccttggagggcatgaagacagatactcctccaaggttattggacaccggaaacaataaattggaacacctcctcaaacctaccactcaggaatgtttgctggggccgaaagaacagtccattgaaagggagtattacaaaaacatggcctttgcttgaaagaaaataccaaggaacaggaaactgatcattaaagcctgagtttgctttcaaa

SEQ ID NO: 46 - protein, 5 aa, Homo sapiens
KSSKC

SEQ ID NO: 47 - protein, 1676 aa, Homo sapiens
MGLLGILCFLIFLGKTWGQEQTYVISAPKIFRVGASENIVIQVYGYTEAFDATISIKSYPDKKFSYSSGHVHLSSENKFQNSAILTIQPKQLPGGQNPVSYVYLEVVSKHFSKSKRMPITYDNGFLFIHTDKPVYTPDQSVKVRVYSLNDDLKPAKRETVLTFIDPEGSEVDMVEEIDHIGIISFPDFKIPSNPRYGMWTIKAKYKEDFSTTGTAYFEVKEYVLPHFSVSIEPEYNFIGYKNFKNFEITIKARYFYNKVVTEADVYITFGIREDLKDDQKEMMQTAMQNTMLINGIAQVTFDSETAVKELSYYSLEDLNNKYLYIAVTVIESTGGFSEEAEIPGIKYVLSPYKLNLVATPLFLKPGIPYPIKVQVKDSLDQLVGGVPVILNAQTIDVNQETSDLDPSKSVTRVDDGVASFVLNLPSGVTVLEFNVKTDAPDLPEENQAREGYRAIAYSSLSQSYLYIDWTDNHKALLVGEHLNIIVTPKSPYIDKITHYNYLILSKGKIIHFGTREKFSDASYQSINIPVTQNMVPSSRLLVYYIVTGEQTAELVSDSVWLNIEEKCGNQLQVHLSPDADAYSPGQTVSLNMATGMDSWVALAAVDSAVYGVQRGAKKPLERVFQFLEKSDLGCGAGGGLNNANVFHLAGLTFLTNANADDSQENDEPCKEILRPRRTLQKKIEEIAAKYKHSVVKKCCYDGACVNNDETCEQRAARISLGPRCIKAFTECCVVASQLRANISHKDMQLGRLHMKTLLPVSKPEIRSYFPESWLWEVHLVPRRKQLQFALPDSLTTWEIQGIGISNTGICVADTVKAKVFKDVFLEMNIPYSVVRGEQIQLKGTVYNYRTSGMQFCVKMSAVEGICTSESPVIDHQGTKSSKCVHQKVEGSSSHLVTFTVLPLEIGLHNINFSLETWFGKEILVKTLRVVPEGVKRESYSGVTLDPRGIYGTISRRKEFPYRIPLDLVPKTEIKRILSVKGLLVGEILSAVLSQEGINILTHLPKGSAEAELMSVVPVFYVFHYLETGNHWNIFHSDPLIEKQKLKKKLKEGMLSIMSYRNADYSYSVWKGGSASTWLTAFALRVLGQVNKYVEQNQNSICNSLLWLVENYQLDNGSFKENSQYQPIKLQGTLPVEARENSLYLTAFTVIGIRKAFDICPLVKIDTALIKADNFLLENTLPAQSTFTLAISAYALSLGDKTHPQFRSIVSALKREALVKGNPPIYRFWKDNLQHKDSSVPNTGTARMVETTAYALLTSLNLKDINYVNPVIKWLSEEQRYGGGFYSTQDTINAIEGLTEYSLLVKQLRLSMDIDVSYKHKGALHNYKMTDKNFLGRPVEVLLNDDLIVSTGFGSGLATVHVTTVVHKTSTSEEVCSFYLKIDTQDIEASHYRGYGNSDYKRIVACASYKPSREESSSGSSHAVMDISLPTGISANEEDLKALVEGVDQLFTDYQIKDGHVILQLNSIPSSDFLCVRFRIFELFEVGFLSPATFTVYEYHRPDKQCTMFYSTSNIKIQKVCEGAACKCVEADCGQMQEELDLTISAETRKQTACKPEIAYAYKVSITSITVENVFVKYKATLLDIYKTGEAVAEKDSEITFIKKVTCTNAELVKGRQYLIMGKEALQIKYNFSFRYIYPLDSLTWIEYWPRDTTCSSCQAFLANLDEFAEDIFLNGC

SEQ ID NO: 48 - protein, 1658 aa, Homo sapiens
QEQTYVISAPKIFRVGASENIVIQVYGYTEAFDATISIKSYPDKKFSYSSGHVHLSSENKFQNSAILTIQPKQLPGGQNPVSYVYLEVVSKHFSKSKRMPITYDNGFLFIHTDKPVYTPDQSVKVRVYSLNDDLKPAKRETVLTFIDPEGSEVDMVEEIDHIGIISFPDFKIPSNPRYGMWTIKAKYKEDFSTTGTAYFEVKEYVLPHFSVSIEPEYNFIGYKNFKNFEITIKARYFYNKVVTEADVYITFGIREDLKDDQKEMMQTAMQNTMLINGIAQVTFDSETAVKELSYYSLEDLNNKYLYIAVTVIESTGGFSEEAEIPGIKYVLSPYKLNLVATPLFLKPGIPYPIKVQVKDSLDQLVGGVPVILNAQTIDVNQETSDLDPSKSVTRVDDGVASFVLNLPSGVTVLEFNVKTDAPDLPEENQAREGYRAIAYSSLSQSYLYIDWTDNHKALLVGEHLNIIVTPKSPYIDKITHYNYLILSKGKIIHFGTREKFSDASYQSINIPVTQNMVPSSRLLVYYIVTGEQTAELVSDSVWLNIEEKCGNQLQVHLSPDADAYSPGQTVSLNMATGMDSWVALAAVDSAVYGVQRGAKKPLERVFQFLEKSDLGCGAGGGLNNANVFHLAGLTFLTNANADDSQENDEPCKEILRPRRTLQKKIEEIAAKYKHSVVKKCCYDGACVNNDETCEQRAARISLGPRCIKAFTECCVVASQLRANISHKDMQLGRLHMKTLLPVSKPEIRSYFPESWLWEVHLVPRRKQLQFALPDSLTTWEIQGIGISNTGICVADTVKAKVFKDVFLEMNIPYSVVRGEQIQLKGTVYNYRTSGMQFCVKMSAVEGICTSESPVIDHQGTKSSKCVHQKVEGSSSHLVTFTVLPLEIGLHNINFSLETWFGKEILVKTLRVVPEGVKRESYSGVTLDPRGIYGTISRRKEFPYRIPLDLVPKTEIKRILSVKGLLVGEILSAVLSQEGINILTHLPKGSAEAELMSVVPVFYVFHYLETGNHWNIFHSDPLIEKQKLKKKLKEGMLSIMSYRNADYSYSVWKGGSASTWLTAFALRVLGQVNKYVEQNQNSICNSLLWLVENYQLDNGSFKENSQYQPIKLQGTLPVEARENSLYLTAFTVIGIRKAFDICPLVKIDTALIKADNFLLENTLPAQSTFTLAISAYALSLGDKTHPQFRSIVSALKREALVKGNPPIYRFWKDNLQHKDSSVPNTGTARMVETTAYALLTSLNLKDINYVNPVIKWLSEEQRYGGGFYSTQDTINAIEGLTEYSLLVKQLRLSMDIDVSYKHKGALHNYKMTDKNFLGRPVEVLLNDDLIVSTGFGSGLATVHVTTVVHKTSTSEEVCSFYLKIDTQDIEASHYRGYGNSDYKRIVACASYKPSREESSSGSSHAVMDISLPTGISANEEDLKALVEGVDQLFTDYQIKDGHVILQLNSIPSSDFLCVRFRIFELFEVGFLSPATFTVYEYHRPDKQCTMFYSTSNIKIQKVCEGAACKCVEADCGQMQEELDLTISAETRKQTACKPEIAYAYKVSITSITVENVFVKYKATLLDIYKTGEAVAEKDSEITFIKKVTCTNAELVKGRQYLIMGKEALQIKYNFSFRYIYPLDSLTWIEYWPRDTTCSSCQAFLANLDEFAEDIFLNGC

(19) United States
(12) Patent Application Publication
(10) Pub. No.: US 2015/0247849 A1
(43) Pub. Date: Sep. 3, 2015

(54) SCREENING ASSAYS FOR COMPLEMENT COMPONENT C5 ANTAGONISTS

(71) Applicants: Paul P. TAMBURINI, (US); ALEXION PHARMACEUTICALS, INC., Cheshire, CT (US)

(72) Inventor: Paul P. Tamburini, Kensington, CT (US)

(21) Appl. No.: 14/428,050

(22) PCT Filed: Sep. 20, 2013
(86) PCT No.: PCT/US2013/061019

§ 371 Date: Mar. 13, 2015

Related U.S. Application Data
(60) Provisional application No. 61/775,156, filed on Mar. 8, 2013.
(60) Provisional application No. 61/704,392, filed on Sep. 21, 2012.

(51) Int. Cl. G01N 33/564

(52) U.S. Cl. CPC G01N 33/564; G01N 2500/04; G01N 2333/4716

(57) ABSTRACT

Disclosed herein are compositions and methods for screening for novel compounds that bind to polypeptides of therapeutic interest (e.g., polypeptides implicated in, or known to contribute to, the pathogenesis of human disease). In some embodiments, the compounds bind to a component of the human complement cascade such as human complement component C5. In some embodiments, the compounds so identified inhibit complement-mediated activity and are potential drug candidates for treating complement-associated disorders. This disclosure also provides compositions and methods for screening for novel compounds that inhibit complement-mediated activity and may be useful for identifying potential drug candidates for treating patients showing little or no response to the existing therapies for treating complement associated disorders.

### CROSS-REFERENCE TO RELATED APPLICATIONS

This application claims priority to and the benefit of U.S. provisional patent application Ser. Nos. 61/704,392 filed on Sep. 21, 2012, and 61/775,156, filed on Mar. 8, 2013, the disclosures of which are incorporated herein by reference in their entirety.

### BACKGROUND

While drug treatment remains a mainstay of medicine, in many cases a given therapeutically efficacious drug may have little or no effect in a portion of a given patient population. In the absence of an alternative therapeutic option, this portion of the patient population may have limited or no alternatives for therapy. Genetic variation in the affected patient population often underlies this lack of responsiveness. Indeed, there exist several examples of such correlations, for example variability in response to various drugs due to polymorphisms in the Cyt P450 gene are well known. Given that these genetic variations may be reflected in differences in regulatory functions of these genes, variability in the mRNAs and/or protein expressions of these genes would be expected. Pharmacogenomics holds the promise that one may soon be able to profile variations between individuals' genetic makeup that accurately predict responses to drugs, addressing both efficacy and safety issues.

### SUMMARY

The present disclosure relates to compositions (e.g., kits) and methods useful for identifying novel compounds that bind to polypeptides of therapeutic interest (e.g., polypeptides implicated in, or known to contribute to, the pathogenesis of human disease). In some embodiments, these novel compounds bind to complement component polypeptides (e.g., C1, C2, C3, C4, C5, C6, C7, C8, C9, MASP1, MASP2, properdin, factor D, factor H, factor I, or factor B) and, in some embodiments, inhibit complement activity (e.g., in vitro and/or in vivo). The novel compounds identified using the methods and compositions described herein are useful for, among other things, treating human disease. For example, complement inhibitory compounds identified using the compositions and methods provided herein are potential drug candidates useful for treating humans afflicted with complement-associated disorders such as, but not limited to, paroxysmal nocturnal hemoglobinuria (PNH) and atypical hemolytic uremic syndrome (aHUS).

The inventor became aware that a small number of patients with PNH did not respond to treatment with eculizumab, which is an antibody that binds to complement component C5 and inhibits cleavage of C5 into fragments C5a and C5b. The inventor reasoned that non-responsiveness may be due to reduced binding, or lack of binding, of eculizumab to C5 in the blood of the treated patients and that the reduced binding, or lack of binding, may be due to a variation in the nucleotide sequence encoding the C5 polypeptide. Variation in the nucleotide sequence of the C5 gene can result in modifications at the protein level within and/or overlapping with the binding pockets and/or epitopes of C5 protein recognized by eculizumab.

In view of this, the inventor conceived of a number of screening methods employing variant forms of wild-type polypeptides of therapeutic interest (e.g., naturally-occurring variant forms of wild-type polypeptides of therapeutic interest to which a known therapeutic agent does not bind or poorly binds) and useful for identifying one or more novel compounds that, e.g.: (a) bind to a wild-type polypeptide, e.g., at a region within or overlapping with the region of the wild-type polypeptide bound by a known therapeutic agent; (b) bind to a variant polypeptide that is not bound, or bound very poorly, by the known wild-type polypeptide antagonist compound; (c) bind to both a wild-type polypeptide and a variant polypeptide (e.g., at a region to which the known therapeutic agent does not bind); (d) bind preferentially to a wild-type polypeptide as compared to a variant polypeptide; (e) bind preferentially to a variant polypeptide as compared to a wild-type polypeptide; (f) bind a wild-type polypeptide, but do not bind to a variant polypeptide; or (g) bind to a variant polypeptide, but do not bind to a wild-type polypeptide. The known therapeutic agent can be a known antagonist or a known agonist of the wild-type polypeptide. In some embodiments the known therapeutic agent is an agent that has been approved by a governmental regulatory authority (e.g., the U.S. Food and Drug Administration or the European Medicines Agency) for the treatment of a human disease or condition or the amelioration of one or more symptoms of a human disease or condition.

The wild-type polypeptide can be any polypeptide of therapeutic interest, e.g., a human polypeptide. In some embodiments, the wild-type polypeptide is a growth factor (e.g., epidermal growth factor, a bone morphogenic protein, erythropoietin, fibroblast growth factor, glial cell-derived neurotrophic factor, granulocyte colony stimulating factor, insulin-like growth factor, myostatin, nerve growth factor, thrombopoietin, platelet-derived growth factor, or vascular endothelial growth factor), a cytokine (e.g., TGFα, TGFβ, IFNα, IFNβ, IFNγ, TNFα, TNFβ, an interleukin such as IL-1, IL-2, IL-3, IL-4, IL-5, IL-6, IL-10, IL-12), or a chemokine (e.g., CCL1, CCL2, CCL3, CCL4, CCL5, CCL6, CCL7, CCL8, CXCL1, CXCL2, CXCL3, CXCL4, CXCL5, CXCL6, CXCL7, CXCL8, CXCL9, CXCL10, etc.). Growth factors can include, e.g., vascular endothelial growth factor (VEGF), insulin-like growth factor (IGF), bone morphogenic protein (BMP), granulocyte-colony stimulating factor (G-CSF), granulocyte-macrophage colony stimulating factor (GM-CSF), nerve growth factor (NGF); a neurotrophin, platelet-derived growth factor (PDGF), erythropoietin (EPO), thrombopoietin (TPO), myostatin (GDF-8), growth differentiation factor-9 (GDF9), basic fibroblast growth factor (bFGF or FGF2), epidermal growth factor (EGF), hepatocyte growth factor (HGF), and a neuregulin (e.g., NRG1, NRG2, NRG3, or NRG4). Cytokines include, e.g., interferons (e.g., IFNγ), tumor necrosis factor (e.g., TNFα or TNFβ), and the interleukins (e.g., IL-1 to IL-33 (e.g., IL-1, IL-2, IL-3, IL-4, IL-5, IL-6, IL-7, IL-8, IL-9, IL-10, IL-12, IL-13, or IL-15)). Chemokines include, e.g., I-309, TCA-3, MCP-I, MIP-1α, MIP-1β, RANTES, C10, MRP-2, MARC, MCP-3, MCP-2, MRP-2, CCF18, Eotaxin, MCP-5, MCP-4, NCC-I, HCC-I, leukotactin-1, LEC, NCC-4, CCL21, TARC, PARC, or Eotaxin-2. In some embodiments, the wild-type polypeptide can be an antibody or portion thereof such as, e.g., the Fc portion of IgM, IgG (including IgG1, IgG2, IgG3, and IgG4), IgA, IgD, or IgE. In some embodiments, the wild-type polypeptide can be a cell surface protein such as, e.g., a G protein coupled receptor (GPCR), a chemokine receptor, a cytokine receptor, or a receptor tyrosine kinase (RTK). The chemokine receptor can be, e.g., CCR1, CCR2, CCR3, CCR4, CCR5, CCR6, CCR7, CCR8, CXCR1, CXCR2, CXCR3, CXCR4, or CCX-CKR2. The cytokine receptors include, e.g., IL-1R, IL-2R, IL-3R, IL-4R, IL-5R, IL-6R, IL-8R, TNFβR1, TNFβR2, c-kit receptor, interferon (IFNα or IFNβ) receptor, IFN gamma receptor, granulocyte macrophage colony stimulating factor (GM-CSF) receptor, granulocyte colony stimulating factor (G-CSF) receptor, and prolactin receptor. RTKs include, e.g., EGF receptor, insulin receptor, PDGF receptor, FGF receptor, VEGF receptor, and HGF receptor.

In some embodiments, the wild-type polypeptide is a complement protein such as, e.g., C1, C1q, C1r, C1s, C4, C4a, C4b, C3, C3a, C3b, C2, C2a, C2b, C5, C5a, C5b, C6, C7, C8, C9, properdin, complement factor B, complement factor D, MBL, MASP1, MASP2, or MASP3.

In some embodiments, the wild-type protein is selected from the group consisting of: ABCF1; ACVR1; ACVR1B; ACVR2; ACVR2B; ACVRL1; ADORA2A; Aggrecan; AGR2; AICDA; AIF1; AIG1; AKAP1; AKAP2; AMH; AMHR2; ANGPT1; ANGPT2; ANGPTL3; ANGPTL4; ANPEP; APC; APOC1; AR; AZGP1 (zinc-α-glycoprotein); B7.1; B7.2; BAD; BAFF; BAG1; BAI1; BCL2; BCL6; BDNF; BLNK; BLR1 (MDR15); BIyS; bone morphogenic protein (BMP) 1; BMP2; BMP3B (GDF10); BMP4; BMP6; BMP8; BMPR1A; BMPR1B; BMPR2; BPAG1 (plectin); BRCA1; BRCA2; C19orf10 (IL27w); complement component C3; complement component C3a; complement component C3b; complement component C4a; complement component C4b; complement component C5; complement component C5a; complement component C5b; complement component C6; complement component C7; complement component C8; complement component C9; complement factor D; complement factor B; C5aR1; CANT1; CASP1; CASP4; CAV1; CCBP2 (D6/JAB61); CCL1 (1-309); CCL11 (eotaxin); CCL13 (MCP-4); CCL15 (MIP-1d); CCL16 (HCC-4); CCL17 (TARC); CCL18 (PARC); CCL19 (MIP-3b); CCL2 (MCP-1); MCAF; CCL20 (MIP-3a); CCL21 (MEP-2); SLC; exodus-2; CCL22 (MDC/STC-I); CCL23 (MPIF-1); CCL24 (MPIF-2/eotaxin-2); CCL25 (TECK); CCL26 (eotaxin-3); CCL27 (CTACK/ILC); CCL28; CCL3 (MIP-1a); CCL4 (MIP-1b); CCL5 (RANTES); CCL7 (MCP-3); CCL8 (mcp-2); CCNA1; CCNA2; CCND1; CCNE1; CCNE2; CCR1 (CKR1/HM145); CCR2 (mcp-1RB); CCR3 (CKR3/CMKBR3); CCR4; CCR5 (CMKBR5/ChemR13); CCR6 (CMKBR6/CKR-L3/STRL22/DRY6); CCR7 (CKR7/EB11); CCR8 (CMKBR8/TER1/CKR-L1); CCR9 (GPR-9-6); CCRL1 (VSHK1); CCRL2 (L-CCR); CD164; CD19; CD1C; CD20; CD200 (OX-2); CD200R; CD-22; CD24; CD28; CD3; CD37; CD38; CD3E; CD3G; CD3Z; CD4; CD40; CD40L; CD44; CD45RB; CD52; CD69; CD72; CD74; CD79A; CD79B; CD8; CD80; CD81; CD83; CD86; CDH1 (E-cadherin); CDH10; CDH12; CDH13; CDH18; CDH19; CDH20; CDH5; CDH7; CDH8; CDH9; CDK2; CDK3; CDK4; CDK5; CDK6; CDK7; CDK9; CDKN1A (p21Wap1/Cip1); CDKN1B (p27Kip1); CDKN1C; CDKN2A (p16INK4a); CDKN2B; CDKN2C; CDKN3; CEBPB; CER1; CHGA; CHGB; chitinase; CHST10; CKLFSF2; CKLFSF3; CKLFSF4; CKLFSF5; CKLFSF6; CKLFSF7; CKLFSF8; CLDN3; CLDN7 (claudin-7); CLN3; CLU (clusterin); CMKLR1; CMKOR1 (RDC1); CNR1; COL18A1; COL1A1; COL4A3; COL6A1; CR2; CRP; CSF1 (M-CSF); CSF2 (GM-CSF); CSF3 (GCSF); CTLA4; CTNNB1 (β-catenin); CTSB (cathepsin B); CX3CL1 (SCYD1); CX3CR1 (V28); CXCL1 (GRO1); CXCL10; CXCL11 (I-TAC/IP-9); CXCL12 (SDF1); CXCL13; CXCL14; CXCL16; CXCL2 (GRO2); CXCL3 (GRO3); CXCL5 (ENA-78/LIX); CXCL6 (GCP-2); CXCL9 (MIG); CXCR3 (GPR9/CKR-L2); CXCR4; CXCR6 (TYMSTR/STRL33/Bonzo); CYB5; CYC1; CYSLTR1; DAB2IP; DES; DKFZp451J0118; DNCL1; DPP4; DR6; E2F1; ECGF1; EDG1; EFNA1; EFNA3; EFNB2; EGF; EGFR; ELAC2; endocan; ENG; ENO1; ENO2; ENO3; EPHB4; EPG; EPO; ERBB2 (Her-2); EREG; ERK8; ESR1; ESR2; F3 (TF); FADD; FasL; FASN; fFCER1A; FCER2; FCGR3A; FGF; FGF1 (aFGF); FGF10; FGF11; FGF12; FGF12B; FGF13; FGF14; FGF16; FGF17; FGF18; FGF19; FGF2 (bFGF); FGF20; FGF21; FGF22; FGF23; FGF3 (int-2); FGF4 (HST); FGF5; FGF6 (HST-2); FGF7 (KGF); FGF8; FGF9; FGFR3; FIGF (VEGFD); FIL1 (EPSILON); FIL1 (ZETA); FLJ12584; FLJ25530; FLRT1 (fibronectin); FLT1; FOS; FOSL1 (FRA-I); FY (DARC); GABRP (GABAa); GAGEB1; GAGEC1; GALNAC4S-6ST; GATA3; GDF5; GFI1; GGT1; GM-CSF; GNAS1; GNRH1; GPR2 (CCR10); GPR31; GPR44; GPR81 (FKSG80); GRCC10 (C10); GRP; GSN (Gelsolin); GSTP1; HAVCR1; HAVCR2; HDAC4; HDAC5; HDAC7A; HDAC9; HGF; HIF1A; HIP1; histamine and histamine receptors; HLA-A; HLA-DRA; HM74; HMOX1; HUMCYT2A; ICEBERG; ICOSL; ID2; IFN-α; IFNA1; IFNA2; IFNA4; IFNA5; IFNA6; IFNA7; IFNB1; IFNγ; IFNW1; IGBP1; IGF1; IGF1R; IGF2; IGFBP2; IGFBP3; IGFBP6; IL-1; IL-10; IL-10RA; IL-10RB; IL11; IL11RA; IL-12; IL12A; IL12B; IL12RB1; IL12RB2; DL13; IL13RA1; IL13RA2; IL14; IL15; IL15RA; IL16; IL17; IL17B; IL17C; IL17R; IL18; IL18BP; IL18R1; IL18RAP; IL19; IL1A; IL1B; IL1F10; IL1F5; IL1F6; IL1F7; IL1F8; IL1F9; IL1HY1; IL1R1; IL1R2; IL1RAP; IL1RAPL1; IL1RAPL2; IL1RL1; IL1RL2; IL1RN; IL2; IL20; IL20RA; IL21R; IL22; IL22R; IL22RA2; IL23; IL24; IL25; IL26; IL27; IL28A; IL28B; IL29; IL2RA; IL2RB; IL2RG; IL3; IL30; IL3RA; IL4; IL4R; IL5; IL5RA; IL6; IL6R; IL6ST (glycoprotein 130); IL7; IL7R; IL8; IL8RA; IL8RB; IL8RB; IL9; IL9R; ILK; INHA; INHBA; INSL3; INSL4; IRAK1; IRAK2; ITGA1; ITGA2; ITGA3; ITGA6 (α6 integrin); ITGAV; ITGB3; ITGB4 (β4 integrin); JAG1; JAK1; JAK3; JUN; K6HF; KAI1; KDR; KITLG; KLF5 (GC Box BP); KLF6; KLK10; KLK12; KLK13; KLK14; KLK15; KLK3; KLK4; KLK5; KLK6; KLK9; KRT1; KRT19 (Keratin 19); KRT2A; KRTHB6 (hair-specific type II keratin); LAMAS; LEP (leptin); Lingo-p75; Lingo-Troy; LPS; LTA (TNF-β); LTB; LTB4R (GPR16); LTB4R2; LTBR; MACMARCKS; MAG or Omgp; MAP2K7 (c-Jun); MDK; MIB1; midkine; MIF; MIP-2; MKI67 (Ki-67); MMP2; MMP9; MS4A1; MSMB; MT3 (metallothionectin-III); MTSS1; MUC1 (mucin); MYC; MYD88; NCK2; neurocan; NFKB1; NFKB2; NGFB (NGF); NGFR; NgR-Lingo; NgR-Nogo66 (Nogo); NgR-p75; NgR-Troy; NME1 (NM23A); NOX5; NPPB; NROB1; NROB2; NR1D1; NR1D2; NR1H2; NR1H3; NR1H4; NR1I2; NR1I3; NR2C1; NR2C2; NR2E1; NR2E3; NR2F1; NR2F2; NR2F6; NR3C1; NR3C2; NR4A1; NR4A2; NR4A3; NR5A1; NR5A2; NR6A1; NRP1; NRP2; NT5E; NTN4; ODZ1; OPRD1; P2RX7; PAP; PART1; PATE; PAWR; PCA3; PCNA; PDGFA; PDGFB; PECAM1; PF4 (CXCL4); PGF; PGR; phosphacan; PIAS2; PIK3CG; PLAU (uPA); PLG; PLXDC1; PPBP (CXCL7); PPID; PR1; PRKCQ; PRKD1; PRL; PROC; PROK2; properdin; PSAP; PSCA; PTAFR; PTEN; PTGS2 (COX-2); PTN; RAC2 (P21Rac2); RARB; RGS1; RGS13; RGS3; RNF110 (ZNF144); ROBO2; S100A2; SCGB1D2 (lipophilin B); SCGB2A1 (mammaglobin 2); SCGB2A2 (mammaglobin 1); SCYE1 (endothelial Monocyte-activating cytokine); SDF2; SERPINA1; SERPINA3; SERPINB5 (maspin); SERPINE1 (PAI-1); SERPINF1; SHBG; SfcAZ; SLA2; SLC2A2; SLC33A1; SLC43A1; SLIT2; SPP1; SPRR1B (Spr1); ST6GAL1; STAB1; STATE; STEAP; STEAP2; TB4R2; TBX21; TCP10; TDGF1; TEK; TGFA; TGFB1; TGFB1I1; TGFB2; TGFB3; TGFBI; TGFBR1; TGFBR2; TGFBR3; TH1L; THBS1 (thrombospondin-1); THBS2; THBS4; THPO; TIE (Tie-1); TIMP3; tissue factor; TLR10; TLR2; TLR3; TLR4; TLR5; TLR6; TLR7; TLR8; TLR9; TNF; TNF-α; TNFAIP2 (B94); TNFAIP3; TNFRSF11A; TNFRSF1A; TNFRSF1B; TNFRSF21; TNFRSF5; TNFRSF6 (Fas); TNFRSF7; TNFRSF8; TNFRSF9; TNFSF10 (TRAIL); TNFSF11 (TRANCE); TNFSF12 (APO3L); TNFSF13 (April); TNFSF13B; TNFSF14 (HVEM-L); TNFSF15 (VEGI); TNFSF18; TNFSF4 (OX40 ligand); TNFSF5 (CD40 ligand); TNFSF6 (FasL); TNFSF7 (CD27 ligand); TNFSF8 (CD30 ligand); TNFSF9 (4-1BB ligand); TOLLIP; a Toll-like receptor; TOP2A (topoisomerase IIa); p53; TPM1; TPM2; TRADD; TRAF1; TRAF2; TRAF3; TRAF4; TRAF5; TRAF6; TREM1; TREM2; TRPC6; TSLP; TWEAK; VEGF; VEGFB; VEGFC; versican; VHL C5; VLA-4; XCL1 (lymphotactin); XCL2; XCR1 (GPR5/CCXCR1); YY1; and ZFPM2.

In some embodiments, the wild-type polypeptide is one from a microbial pathogen (e.g., virus, bacterium, protozoon, or parasite), which proteins can include, e.g., tetanus toxin, diphtheria toxin, or any of a variety of viral surface proteins (e.g., cytomegalovirus (CMV) glycoproteins B, H and gCIII; human immunodeficiency virus 1 (HIV-I) envelope glycoproteins; Rous sarcoma virus (RSV) envelope glycoproteins; herpes simplex virus (HSV) envelope glycoproteins; Epstein Barr virus (EBV) envelope glycoproteins; varicella-zoster virus (VZV) envelope glycoproteins; human papilloma virus (HPV) envelope glycoproteins; influenza virus glycoproteins; and Hepatitis virus family surface antigens).

The known therapeutic agent (e.g., agonist or antagonist) can be, without limitation, a peptide, a protein (e.g., an antibody), a small molecule, a nucleic acid, or any combination thereof. In some embodiments, the wild-type polypeptide can be the target of any one of the following known therapeutic agents: abagovomab, afelimomab, anatumomab mafenatox, arcitumomab, bectumomab, besilesomab, capromab, edobacomab, edrecolomab, elsilimomab, enlimomab, enlimomab pegol, epitumomab cituxetan, ibritumomab tiuxetan, imciromab, inolimomab, mitumomab, oregovmab, satumomab, sulesomab, technetium (^{99m}Tc) nofetumomab merpentan, tositurnomab, vepalimomab, zolimomab aritox, adalimumab, adecatumumab, belimumab, bertilimumab, denosumab, efungumab, golimumab, ipilimumab, iratumumab, lerdelimumab, lexatumumab, mapatumumab, metelimumab, ofatumumab, panitumumab, pritumumab, raxibacumab, sevirumab, stamulumab, ticilimumab, tuvirumab, votumumab, zalutumumab, zanolimumab, abciximab, basiliximab, bavituximab, cetuximab, ecromeximab, galiximab, infliximab, keliximab, lumiliximab, pagibaximab, priliximab, rituximab, teneliximab, volociximab, alemtuzumab, apolizumab, aselizumab, bapineuzumab, bevacizumab, bivatuzumab, cantuzumab mertansine, certolizumab pegol, daclizumab, eculizumab, efalizurnab, epratuzumab), fontolizumab, gemtuzumab, inotuzumab ozogamicin, labetuzumab, lintuzumab, matuzumab, mepolizumab, motavizumab, natalizumab, nimotuzumab, ocrelizumab, omalizumab, palivizumab, pascolizumab, pertuzumab, pexelizumab, ranibizumab, reslizumab, rovelizumab, ruplizumab, sibrotuzumab, siplizumab, sontuzumab, tadocizumab, talizumab, tefibazumab, tocilizumab, toralizumab, trastuzumab, tucotuzumab celmoleukin, urtoxazumab, visilizumab, or yttrium 90Y tacatuzumab tetraxetan.

In some embodiments, the known therapeutic agent can be, e.g., one selected from the group consisting of, e.g., rituximab (Rituxan®, IDEC/Genentech/Roche), a chimeric anti-CD20 antibody approved to treat Non-Hodgkin's lymphoma; HuMax-CD20, an anti-CD20 currently being developed by Genmab; AME-133 (Applied Molecular Evolution); hA20 (Immunomedics, Inc.); HumaLYM (Intracel); PRO70769 (International patent application no. PCT/US2003/040426); trastuzumab (Herceptin®, Genentech), a humanized anti-Her2/neu antibody approved to treat breast cancer; pertuzumab (rhuMab-2C4, Omnitarg®), currently being developed by Genentech; cetuximab (Erbitux®, Imclone); ABX-EGF currently being developed by Abgenix-Immunex-Amgen; HuMax-EGFr, currently being developed by Genmab; 425, EMD55900, EMD62000, and EMD72000 (Merck KGaA) (see U.S. Pat. No. 5,558,864; Murthy et al. (1987) Arch Biochem Biophys 252(2):549-60; Rodeck et al. (1987) J Cell Biochem 35(4):315-20; and Kettleborough et al. (1991) Protein Eng 4(7):773-83); ICR62 (Institute of Cancer Research) (International publication no. WO 95/20045; Modjtahedi et al. (1993) J Cell Biophys 22(1-3):129-46; Modjtahedi et al. (1993) Br J Cancer 67(2):247-53; Modjtahedi et al. (1996) Br J Cancer 73(2):228-35; Modjtahedi et al. (2003) Int J Cancer 105(2):273-80); TheraCIM hR3 (YM Biosciences, Canada and Centro de Immunologia Molecular, Cuba (U.S. Pat. No. 5,891,996; U.S. Pat. No. 6,506,883; Mateo et al. (1997) Immunotechnology 3(1):71-81)); mAb-806 (Ludwig Institute for Cancer Research, Memorial Sloan-Kettering) (Jungbluth et al. (2003) Proc Natl Acad Sci USA 100(2):639-44); KSB-102 (KS Biomedix); MR1-I (IVAX, National Cancer Institute) (PCT WO 0162931A2); alemtuzumab (Campath®, Millenium), a humanized monoclonal antibody currently approved for treatment of B-cell chronic lymphocytic leukemia; muromonab-CD3 (Orthoclone OKT3®), an anti-CD3 antibody developed by Ortho Biotech/Johnson & Johnson; ibritumomab tiuxetan (Zevalin®), an anti-CD20 antibody developed by IDEC/Schering AG; gemtuzumab ozogamicin (Mylotarg®), an anti-CD33 (p67 protein) antibody developed by Celltech/Wyeth; alefacept (Amevive®), an anti-LFA-3 Fc fusion developed by Biogen; abciximab (ReoPro®), developed by Centocor/Lilly; basiliximab (Simulect®), developed by Novartis; palivizumab (Synagis®), developed by Medimmune; infliximab (Remicade®), an anti-TNFα antibody developed by Centocor; adalimumab (Humira®), an anti-TNFα antibody developed by Abbott; Humicade®, an anti-TNFα antibody developed by Celltech; golimumab (CNTO-148), a fully human anti-TNF antibody developed by Centocor; an anti-CD147 antibody being developed by Abgenix; ABX-IL8, an anti-IL8 antibody being developed by Abgenix; ABX-MA1, an anti-MUC18 antibody being developed by Abgenix; pemtumomab (R1549, ⁹⁰Y-muHMFG1), an anti-MUC1 in development by Antisoma; Therex (R1550), an anti-MUC1 antibody being developed by Antisoma; AngioMab (AS 1405), being developed by Antisoma; HuBC-I, being developed by Antisoma; Thioplatin (AS 1407) being developed by Antisoma; Antegren® (natalizumab) being developed by Biogen Idec and Elan; CAT-152, an anti-TGF-β2 antibody being developed by Cambridge Antibody Technology; ABT 874 (J695), an anti-IL-12 p40 antibody being developed by Abbott; CAT-192, an anti-TGFβ1 antibody being developed by Cambridge Antibody Technology and Genzyme; CAT-213, an anti-Eotaxin1 antibody being developed by Cambridge Antibody Technology; LymphoStat-B®, an anti-Blys antibody being developed by Cambridge Antibody Technology and Human Genome Sciences Inc.; TRAIL-RI mAb, an anti-TRAIL-R1 antibody being developed by Cambridge Antibody Technology and Human Genome Sciences, Inc.; Avastin® (bevacizumab, rhuMAb-VEGF), an anti-VEGF antibody being developed by Genentech; Xolair® (Omalizumab), an anti-IgE antibody being developed by Genentech; Raptiva® (Efalizumab), an anti-CD11a antibody being developed by Genentech and Xoma; MLN-02 Antibody (formerly LDP-02), being developed by Genentech and Millennium Pharmaceuticals; HuMax CD4, an anti-CD4 antibody being developed by Genmab; HuMax-EL15, an anti-IL-15 antibody being developed by Genmab and Amgen; HuMax-Inflam, being developed by Genmab and Medarex, HuMax-Cancer; HuMax-Lymphoma, being developed by Genmab and Amgen; HuMax-TAC, being developed by Genmab; DDEC-131, an anti-CD40L antibody being developed by IDEC Pharmaceuticals; IDEC-151 (Clenoliximab), an anti-CD4 antibody being developed by IDEC Pharmaceuticals; BDEC-114, an anti-CD80 antibody being developed by IDEC Pharmaceuticals; IDEC-152, an anti-CD23 being developed by IDEC Pharmaceuticals; BEC2, an anti-idiotypic antibody being developed by Imclone; IMC-1C11, an anti-KDR antibody being developed by Imclone; DC101, an anti-flk-1 antibody being developed by Imclone; anti-VE cadherin antibodies being developed by Imclone; CEA-Cide® (labetuzumab), an anti-carcinoembryonic antigen (CEA) antibody being developed by Immunomedics; LymphoCide® (Epratuzumab), an anti-CD22 antibody being developed by Immunomedics; AFP-Cide, being developed by Immunomedics; MyelomaCide, being developed by Immunomedics; LkoCide, being developed by Immunomedics; ProstaCide, being developed by Immunomedics; MDX-010, an anti-CTLA4 antibody being developed by Medarex; MDX-060, an anti-CD30 antibody being developed by Medarex; MDX-070 being developed by Medarex; MDX-018 being developed by Medarex; Osidem® (IDM-I), an anti-Her2 antibody being developed by Medarex and Immuno-Designed Molecules; HuMax®-CD4, an anti-CD4 antibody being developed by Medarex and Genmab; HuMax-IL15, an anti-EL15 antibody being developed by Medarex and Genmab; CNTO 148, an anti-TNFα antibody being developed by Medarex and Centocor/Johnson & Johnson; CNTO 1275, an anti-cytokine antibody being developed by Centocor/Johnson & Johnson; MOR101 and MOR102, anti-intercellular adhesion molecule-1 (ICAM-I) (CD54) antibodies being developed by MorphoSys; MOR201, an anti-fibroblast growth factor receptor 3 (FGFR-3) antibody being developed by MorphoSys; Nuvion® (visilizumab), an anti-CD3 antibody being developed by Protein Design Labs; HuZAF®, an anti-gamma interferon antibody being developed by Protein Design Labs; Anti-α5β1 Integrin antibody, being developed by Protein Design Labs; ING-I, an anti-EpCAM antibody being developed by Xoma; Xolair® (Omalizumab) a humanized anti-IgE antibody developed by Genentech and Novartis; and MLNO1, an anti-β2 integrin antibody being developed by Xoma.

As noted above, the inventor conceived of numerous screening methods that employ variant forms of wild-type polypeptides of therapeutic interest and are useful for, among other things, identifying one or more novel compounds that bind to a wild-type polypeptide at a region within or overlapping with the region of the wild-type polypeptide bound by a known therapeutic agent. The inventor recognized that such a method is particularly useful for obtaining one or more compounds that bind to the same site (or a site that substantially overlaps with the site) in human complement component C5 that is bound by eculizumab. In particular, the inventor appreciated that through such methods one could identify compounds that bind to the same site as eculizumab (and thus enjoy the benefits of targeting the eculizumab binding site and emulates the eculizumab activity), but offer greater ease of administration and oral bioavailability, e.g., as in the case of small molecule compounds. Thus, the methods described herein are useful for identifying new compounds that bind to the same site (or a site substantially overlapping with the site) bound by known a therapeutic agent (e.g., a therapeutic antibody such as any of those described herein), which compounds possess one or more improved properties as compared to the known therapeutic agents (e.g., ease of administration, oral bioavailability, improved pharmacokinetics, higher therapeutic index, increased solubility). In other words, the methods can identify improved agonists or antagonists that bind to the same site or a site overlapping with the site bound by known agonists or antagonists of the wild-type form of the polypeptide.

Accordingly, in one aspect, the disclosure features a method for identifying a compound that binds to a wild-type polypeptide at a region within or overlapping with the region of the wild-type polypeptide bound by a known agonist or antagonist of the wild-type polypeptide. The method includes: (i) providing a wild-type polypeptide to which a known agonist or antagonist compound binds; (ii) providing a variant form of the wild-type polypeptide (variant polypeptide) to which the agonist or antagonist: (a) does not bind or (b) binds with lower affinity as compared to the affinity of the known agonist or antagonist for the wild-type polypeptide; (iii) determining whether a test compound binds to the variant polypeptide; and (iv) determining whether the test compound binds to the wild-type polypeptide; wherein a test compound that binds to the wild-type polypeptide, but not to the variant polypeptide, or a test compound that preferentially binds to the wild-type polypeptide as compared to the variant polypeptide is indicative of a compound that binds to the wild-type polypeptide at a region within or overlapping with the region of the wild-type polypeptide bound by the known agonist or antagonist.

In another aspect, the disclosure features a method of screening for a compound that binds to a wild-type polypeptide at a region within or overlapping with the region of the wild-type polypeptide bound by a known agonist or antagonist of the wild-type polypeptide, which method comprises: (i) providing a wild-type polypeptide to which a known agonist or antagonist compound binds; (ii) providing a variant form of the wild-type polypeptide (variant polypeptide) to which the agonist or antagonist: (a) does not bind or (b) binds with lower affinity as compared to the affinity of the known agonist or antagonist for the wild-type polypeptide; (iii) providing a library of test compounds; (iv) screening a plurality of the test compounds for binding to the variant polypeptide; (v) screening a plurality of the test compounds for binding to the wild-type polypeptide; and (vi) selecting one or more test compounds that bind to the wild-type polypeptide, but not to the variant polypeptide or that preferentially bind to the wild-type polypeptide as compared to the variant polypeptide, wherein such compounds are indicative of compounds that bind to the wild-type polypeptide at a region within or overlapping with the region of the wild-type polypeptide bound by the known agonist or antagonist.

Novel compounds identified using methods described herein can be useful as therapeutics, e.g., in patients for which the known therapeutic agent is not effective (e.g., the known therapeutic agent does not bind to its polypeptide target because of a naturally-occurring variation in the amino acid sequence of the target biological polypeptide in those patients). For example, the method and compositions described herein can be used to identify novel therapeutic compounds that bind to variant forms of wild-type polypeptides or bind to both the variant polypeptides and the wild-type polypeptides.

Accordingly, in yet another aspect, the disclosure features a method of identifying a compound that binds to a variant polypeptide at a region within or overlapping with the region of the wild-type form of the polypeptide bound by a known agonist or antagonist of the wild-type polypeptide, the method comprising: (i) providing a wild-type polypeptide to which a known agonist or antagonist compound binds; (ii) providing a variant form of the wild-type polypeptide (variant polypeptide) to which the agonist or antagonist: (a) does not bind or (b) binds with lower affinity as compared to the affinity of the known agonist or antagonist for the wild-type polypeptide; (iii) determining whether a test compound binds to the variant polypeptide; and (iv) determining whether the test compound binds to the wild-type polypeptide; wherein a test compound that binds to the variant polypeptide, but not to the wild-type polypeptide, or a test compound that preferentially binds to the variant polypeptide as compared to the wild-type polypeptide is indicative of a compound that binds to the variant polypeptide at a region within or overlapping with the region of the wild-type polypeptide bound by the known agonist or antagonist.

In yet another aspect, the disclosure features a method of selecting a compound that binds to a wild-type polypeptide and a variant form of the wild-type polypeptide, the method comprising: (i) providing a wild-type polypeptide to which a known agonist or antagonist compound binds; (ii) providing a variant form of the wild-type polypeptide (variant polypeptide) to which the agonist or antagonist: (a) does not bind or (b) binds with lower affinity as compared to the affinity of the known agonist or antagonist for the wild-type polypeptide; (iii) determining whether a test compound binds to the variant polypeptide; (iv) determining whether the test compound binds to the wild-type polypeptide; and (v) selecting the test compound if it binds to the wild-type polypeptide and the variant polypeptide.

In another aspect, the disclosure features a method of screening for a compound that binds to a wild-type polypeptide and a variant form of the wild-type polypeptide. The method includes: (i) providing a wild-type polypeptide to which a known agonist or antagonist compound binds; (ii) providing a variant form of the wild-type polypeptide (variant polypeptide) to which the agonist or antagonist: (a) does not bind or (b) binds with lower affinity as compared to the affinity of the known agonist or antagonist for the wild-type polypeptide; (iii) providing a library of test compounds; (iv) screening a plurality of the test compounds for binding to the variant polypeptide; (v) screening a plurality of the test compounds for binding to the wild-type polypeptide; and (vi) selecting one or more test compounds that bind to the wild-type polypeptide and the variant polypeptide.

In yet another aspect, the disclosure features a method of screening for a compound that binds to a variant form of a wild-type polypeptide at a region within or overlapping with the region of the wild-type polypeptide bound by a known agonist or antagonist of the wild-type polypeptide, the method comprising: (i) providing a wild-type polypeptide to which a known agonist or antagonist compound binds; (ii) providing a variant form of the wild-type polypeptide (variant polypeptide) to which the agonist or antagonist: (a) does not bind or (b) binds with lower affinity as compared to the affinity of the known agonist or antagonist for the wild-type polypeptide; (iii) providing a library of test compounds; (iv) screening a plurality of the test compounds for binding to the variant polypeptide; (v) screening a plurality of the test compounds for binding to the wild-type polypeptide; and (vi) selecting one or more test compounds that bind to the variant polypeptide, but not to the wild-type polypeptide or that preferentially bind to the variant polypeptide as compared to the wild-type polypeptide, wherein such compounds are indicative of compounds that bind to the variant polypeptide at a region within or overlapping with the region of the wild-type polypeptide bound by the known agonist or antagonist.

In some embodiments of any of the methods described herein, the methods further comprise generating the variant polypeptide. Generation of the variant polypeptide can include, e.g., molecular biology techniques in which one or more amino acids of the wild-type form of the polypeptide are substituted for another amino acid, deleted, or inserted into the wild-type polypeptide. In some embodiments, the variant polypeptide comprises no more than 10 (e.g., no more than nine, eight, seven, six, five, four, three, two, or one) amino acid substitution, deletion, or insertion relative to the wild-type form of the polypeptide.

In some embodiments of any of the methods described herein, determining whether the test compound binds to the variant polypeptide comprises determining the binding affinity of the test compound for the variant polypeptide. In some embodiments of any of the methods described herein, determining whether the test compound binds to the wild-type polypeptide comprises determining the binding affinity of the test compound for the wild-type polypeptide. The binding affinity can be determined, e.g., by surface plasmon resonance (SPR), biolayer interferometry, or mass spectrometry.

In some embodiments of any of the methods described herein, the wild-type polypeptide can be a full-length, mature, processed form of the polypeptide (e.g., the mature, processed form of C5) or a fragment of the polypeptide that retains at least 60 (e.g., at least 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99) % of the full-length, mature, processed form of the polypeptide to bind to the known agonist or antagonist.

In some embodiments of any of the methods described herein, the test compound is a small molecule. In some embodiments of any of the methods described herein, more than one test compound is subjected to the method. For example, a library of small molecule test compounds can be subjected to the method, e.g., concurrently (e.g., in various alternative wells of a multi-well assay plate) or successively (e.g., one after another or in small groups).

In some embodiments of any of the methods described herein, the wild-type polypeptide is a human polypeptide. The wild-type polypeptide can be, e.g., a growth factor, a cytokine, or a chemokine. The wild-type polypeptide can be, e.g., a growth factor receptor polypeptide or a growth factor-binding fragment thereof, a cytokine receptor polypeptide or a cytokine-binding fragment thereof, or a chemokine receptor polypeptide or a chemokine-binding fragment thereof. The wild-type polypeptide can be, e.g., a cell surface receptor overexpressed or deregulated in cancer (e.g., HER2/neu). In some embodiments, the wild-type polypeptide can be a polypeptide implicated in or known to be associated with human disease (e.g., transthyretin). In some embodiments of any of the methods described herein, the wild-type polypeptide is a component of the complement cascade. The component of the complement cascade can be, e.g., one selected from the group consisting of C1, C1r, C1s, C1q, C2, C3, C3a, C3b, C4, C4a, C4b, C5, C5a, C5b, C6, C7, C8, C9, MASP1, MASP2, properdin, factor D, factor B, factor H, and factor I.

In some embodiments of any of the methods described herein, the known agonist or antagonist is an approved drug for treating human disease. The known agonist or antagonist can be, e.g., a small molecule, a polypeptide (an antibody or antigen-binding fragment thereof), a polypeptide analog, a peptidomimetic, or an aptamer.

In some embodiments of any of the methods described herein (e.g., in embodiments in which the wild-type polypeptide is a component of the complement cascade), the test compound inhibits cleavage of C5 into fragments C5a and C5b. In some embodiments of any of the methods described herein, the methods can include determining whether the test compound inhibits the cleavage of C5 into fragments C5a and C5b, e.g., using a hemolytic assay.

In some embodiments of any of the methods described herein, the wild-type polypeptide is a wild-type C5 polypeptide. The wild-type C5 polypeptide can comprise, e.g., an amino acid sequence set forth in SEQ ID NO:2 or a fragment thereof. The variant form of the wild-type C5 polypeptide can comprise one or more deletions, insertions, or substitutions relative to the wild-type polypeptide. The variant C5 polypeptide can, e.g., comprise a deletion, insertion, or substitution at a C5 convertase-binding site. The deletion, insertion, or substitution can be present, e.g., between amino acid residues 872 and 892 of SEQ ID NO:2. The deletion, insertion, or substitution can be, in some embodiments of any of the methods described herein, present at the eculizumab-binding epitope.

In some embodiments of any of the methods described herein, the variant C5 polypeptide comprises, or consists of, the amino acid sequence depicted in SEQ ID NO:2 in which the arginine at position 885 is substituted by histidine (R885H). In some embodiments, the variant C5 polypeptide comprises or consists of the amino acid sequence depicted in SEQ ID NO:47 or 48. In some embodiments of any of the methods described herein, the variant C5 polypeptide comprises at least five (e.g., at least six, seven, eight, nine, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 125, 150, 175, 200, 250, 300, 350, 400, 450, 500, 550, 600, 650, 700, 750, 800, or 850) consecutive amino acids of SEQ ID NO:47 inclusive of histidine 885. In some embodiments, the variant C5 polypeptide: (a) comprises at least 20 (e.g., at least 21, 22, 23, 24, 25, 26, 27, 28, 29, 30, 35, 40, 45, 50, 55, 60, 65, 70, 75, 80, 85, 90, 95, 100, 125, 150, 175, 200, 250, 300, 350, 400, 450, 500, 550, 600, 650, 700, 750, 800, or 850) amino acids, (b) is at least 80% identical to a corresponding at least 20 amino acid sequence of SEQ ID NO:47, and (c) comprises histidine 885 of SEQ ID NO:47.

In some embodiments of any of the methods described herein, the variant form of the wild-type polypeptide can be present in subjects non-responsive to treatment with the known antagonist or agonist. For example, in some embodiments, the variant C5 polypeptide is present in a population of patients non-responsive to eculizumab.

In some embodiments of any of the methods described herein, the known antagonist is a known complement component C5 antagonist. The known wild-type C5 antagonist can be, e.g., eculizumab, pexelizumab, MB12/22, MB12/22-RGD, ARC187, ARC1905, SSL7, or OmCI.

In some embodiments of any of the methods described herein, determining whether the test compound binds to the variant polypeptide or the wild-type polypeptide is performed by surface plasmon resonance, biolayer interferometry, mass spectrometry, or immunoassay such as an enzyme-linked immunosorbent assay (ELISA) or a radioimmunoassay (RIA).

In some embodiments of any of the methods described herein, the test compound can be, e.g., one selected from the group consisting of an antibody (or an antigen-binding fragment thereof), a small molecule, a polypeptide, a polypeptide analog, a peptidomimetic, a nucleic acid, a nucleic acid analog, and an aptamer.

In some embodiments of any of the methods described herein, the test compound can be one rationally designed to bind to the wild-type polypeptide, e.g., at a site bound by a known agonist or antagonist. In some embodiments, the test compound is rationally designed to bind the wild-type C5 polypeptide. The test compound can be rationally designed to bind a C5 convertase-binding site of C5, e.g., the test compound is rationally designed to bind an epitope of C5 set forth between or comprising residues 872 and 892 of SEQ ID NO:2 and/or a C5 convertase-cleavage site of C5. In some embodiments, the test compound is rationally designed to bind to an epitope of C5 set forth between or comprising residues 872 and 892 of SEQ ID NO: 2 or 47, e.g., at least five (e.g., at least six, seven, eight, nine, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 125, 150, 175, 200, 250, 300, 350, 400, 450, 500, 550, 600, 650, 700, 750, 800, or 850) consecutive amino acids of SEQ ID NO:2 or 47, inclusive of amino acid 885.

In another aspect, the disclosure features a method of identifying a compound that binds to a wild-type polypeptide at a region within or overlapping with the region of the wild-type polypeptide bound by a known therapeutic agent (e.g., an antagonist or agonist), which method includes: (i) providing a variant polypeptide to which a known therapeutic agent: (a) does not bind or (b) binds with lower affinity as compared to the affinity of the known therapeutic agent for the corresponding wild-type form of the polypeptide (the wild-type polypeptide); (ii) determining whether a test compound binds to the variant polypeptide; and (iii) determining whether the test compound binds to the wild-type polypeptide; wherein a test compound that binds to the wild-type polypeptide, but not to the variant polypeptide or a test compound that preferentially binds to the wild-type polypeptide as compared to the variant polypeptide, is indicative of a compound that binds to the wild-type polypeptide at a region within or overlapping with the region of the wild-type polypeptide bound by the known therapeutic agent.

In another aspect, the disclosure features a method of identifying a compound that binds to a variant polypeptide at a region within or overlapping with the corresponding region of the wild-type polypeptide bound by a known therapeutic agent (e.g., an antagonist or agonist). The method includes: (i) providing a variant polypeptide to which a known therapeutic agent: (a) does not bind or (b) binds with lower affinity as compared to the affinity of the known therapeutic agent for the corresponding wild-type form of the polypeptide (the wild-type polypeptide); (ii) determining whether a test compound binds to the variant polypeptide; and (iii) determining whether the test compound binds to the wild-type polypeptide; wherein a test compound that binds to the variant polypeptide, but not to the wild-type polypeptide or a test compound that preferentially binds to the variant polypeptide as compared to the wild-type polypeptide, is indicative of a compound that binds to the variant polypeptide at a region within or overlapping with the corresponding region of the wild-type polypeptide bound by the known therapeutic agent.

In yet another aspect, the disclosure features a method of identifying a compound that binds to polypeptide of interest at a region that is not within or overlapping with the region of a wild-type polypeptide of therapeutic interest bound by a known therapeutic agent (e.g., an antagonist or agonist). The method includes: (i) providing a variant polypeptide to which a known therapeutic agent: (a) does not bind or (b) binds with lower affinity as compared to the affinity of the known therapeutic agent for the corresponding wild-type form of the polypeptide (the wild-type polypeptide); (ii) determining whether a test compound binds to the variant polypeptide; and (iii) determining whether the test compound binds to the wild-type polypeptide; wherein a test compound that binds to the variant polypeptide and the wild-type polypeptide is indicative of a compound that binds to the wild-type polypeptide and variant polypeptide at a region that is not within or overlapping with the region of the wild-type polypeptide bound by the known therapeutic agent.

In some embodiments, the polypeptide of therapeutic interest is a complement component polypeptide (e.g., C5). In some embodiments, the known therapeutic agent is eculizumab.

In some embodiments, preferential binding of a test compound to one polypeptide over another is at least a two fold difference in affinity. In some embodiments, preferential binding is at least a three (e.g., at least a four, five, six, seven, eight, nine, 10, 20, 40, 50, 100, 500, 1000, 2000, 5000, 10000) fold difference in affinity between the test compound for one polypeptide and the test compound for another polypeptide.

In certain aspects, the disclosure provides a method of identifying a compound that inhibits cleavage of C5 into C5a and C5b, the method comprising (i) determining the binding affinity of a test compound to a wild-type C5 polypeptide, (ii) determining the binding affinity of the test compound to a variant C5 polypeptide, and (iii) comparing the binding affinity of the test compound to the wild-type C5 polypeptide to the binding affinity of the test compound to the variant C5 polypeptide, wherein greater affinity of the test compound for the wild-type C5 polypeptide is indicative of a compound that inhibits cleavage of the wild-type C5 polypeptide.

In some embodiments, the method further comprises testing the test compound in a complement-mediated hemolysis assay to determine if it inhibits complement mediated hemolysis wherein a test compound that further inhibits complement-mediated hemolysis is identified as a compound that inhibits cleavage of C5 into C5a and C5b.

In some embodiments, the wild-type C5 polypeptide comprises an amino acid sequence set forth in SEQ ID NO:2 or a fragment thereof.

In some embodiments, the variant C5 polypeptide comprises a deletion, an insertion, or a substitution. In some embodiments, the deletion, insertion, or substitution is at a C5 convertase-binding site. In some embodiments, the deletion, insertion, or substitution is present between or comprising residues 872 and 892 of SEQ ID NO:2, e.g., amino acid residue 885 of SEQ ID NO:2. In some embodiments, the arginine at amino acid position 885 is substituted for a histidine. In some embodiments, the variant C5 polypeptide comprises all or at least five (e.g., at least six, seven, eight, nine, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 125, 150, 175, 200, 250, 300, 350, 400, 450, 500, 550, 600, 650, 700, 750, 800, or 850) consecutive amino acids of SEQ ID NO:47, inclusive of amino acid residue 885. In some embodiments, the deletion, insertion, or substitution is present at the eculizumab-binding epitope. In some embodiments, the variant C5 polypeptide is present in subjects non-responsive to treatment with a known C5 antagonist.

In some embodiments, the known C5 antagonist is an anti-C5 antibody or an antigen binding fragment thereof, a small molecule, a polypeptide, a polypeptide analog, a peptidomimetic, or an aptamer. In some embodiments, the known C5 antagonist is eculizumab. In some embodiments, the known C5 antagonist is pexelizumab. In some embodiments, the known C5 antagonist is selected from the group consisting of MB12/22, MB12/22-RGD, ARC187, ARC1905, SSL7, and OmCI.

In some embodiments, the binding affinity is determined by surface plasmon resonance. In some embodiments, the binding affinity is determined by biolayer interferometry. In some embodiments, the binding affinity is determined by mass spectrometry. In some embodiments, the binding affinity is determined by an immunoassay. In some embodiments, the immunoassay is an enzyme-linked immunosorbent assay (ELISA). In some embodiments, the immunoassay is a radioimmunoassay (RIA).

In some embodiments, the test compound is selected from: an antibody, a small molecule, a polypeptide, a polypeptide analog, a peptidomimetic, a nucleic acid, a nucleic acid analog, and an aptamer. In some embodiments, the test compound is present in a library. In some embodiments, the test compound is rationally designed to bind the wild-type C5 polypeptide. In some embodiments, the test compound is rationally designed to bind a C5 convertase-binding site of C5. In some embodiments, the test compound is rationally designed to bind an epitope of C5 set forth between or comprising residues 872 and 892 of SEQ ID NO:2 or 47, e.g., a polypeptide comprising at least five (e.g., at least six, seven, eight, nine, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 125, 150, 175, 200, 250, 300, 350, 400, 450, 500, 550, 600, 650, 700, 750, 800, or 850) consecutive amino acids of SEQ ID NO:2 or 47, inclusive of amino acid 885 In some embodiments, the test compound is designed to bind a C5 convertase-cleavage site of C5. In some embodiments, the test compound is designed to bind to a site on C5 known to be bound by an inhibitor of cleavage of C5.

In certain aspects, the disclosure provides an assay for identifying a potential complement inhibitor for treating C5-associated disorders, the assay comprising (i) determining binding of a test compound to a wild-type C5 polypeptide, and (ii) determining binding of the test compound to a variant C5 polypeptide, wherein a test compound that binds the wild-type C5 polypeptide but not the variant C5 polypeptide is a potential complement inhibitor for treating a C5-associated disorder.

In some embodiments, the method further comprises (iii) testing the test compound in a complement-mediated hemolysis assay to determine if it inhibits complement mediated hemolysis, wherein a test compound that inhibits complement-mediated hemolysis is identified as a complement inhibitor.

In some embodiments, the variant polypeptide has a mutation in the range of residues 872-892 of SEQ ID NO:2, e.g., amino acid position 885 of SEQ ID NO:2.

In some embodiments, the C5-associated disorder is selected from the group comprising paroxysmal nocturnal hemoglobinuria (PNH), atypical hemolytic-uremic syndrome (aHUS), shiga toxin E. coli-related hemolytic uremic syndrome (STEC-HUS), dense deposit disease (DDD), C3 nephropathy, myasthenia gravis, neuromyelitis optica, cold agglutinin disease (CAD), antineutrophil cytoplasm antibody (ANCA)-associated vasculitis (AAV), asthma, age-related macular degeneration (AMD), transplant rejection, Goodpasture's syndrome, glomerulonephritis, vasculitis, rheumatoid arthritis, dermatitis, systemic lupus erythematosus (SLE), Guillain-Barr syndrome (GBS), dermatomyositis, psoriasis, Graves' disease, Hashimoto's thyroiditis, type I diabetes, pemphigus, autoimmune hemolytic anemia (AIHA), idiopathic thrombocytopenic purpura (ITP), lupus nephritis, ischemia-reperfusion injury, thrombotic thrombocytopenic purpura (TTP), Pauci-immune vasculitis, epidermolysis bullosa, multiple sclerosis, spontaneous fetal loss, recurrent fetal loss, traumatic brain injury, injury resulting from myocardial infarction, cardiopulmonary bypass and hemodialysis, and hemolysis, elevated liver enzymes, and low platelets (HELLP) syndrome. In some embodiments, the C5-associated disorder is PNH. In some embodiments, the C5-associated disorder is aHUS.

In some embodiments, the wild-type C5 polypeptide comprises an amino acid sequence set forth in SEQ ID NO:2 or a fragment thereof.

In some embodiments, the variant C5 polypeptide comprises a deletion, an insertion, or a substitution. In some embodiments, the deletion, insertion, or substitution is at a C5 convertase-binding site. In some embodiments, the deletion, insertion, or substitution is present between or inclusive of residues 872 and 892 of SEQ ID NO:2, e.g., the variant polypeptide can comprise or consist of at least five (e.g., at least six, seven, eight, nine, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 125, 150, 175, 200, 250, 300, 350, 400, 450, 500, 550, 600, 650, 700, 750, 800, or 850) consecutive amino acids of SEQ ID NO:47, inclusive of amino acid 885.

In some embodiments, the deletion, insertion, or substitution is present at the eculizumab-binding epitope. In some embodiments, the variant C5 polypeptide is present in subjects non-responsive to treatment with a known C5 antagonist.

In some embodiments, the known C5 antagonist is an anti-C5 antibody or an antigen binding fragment thereof, a small molecule, a polypeptide, a polypeptide analog, a peptidomimetic, or an aptamer. In some embodiments, the known C5 antagonist is eculizumab. In some embodiments, the known C5 antagonist is pexelizumab. In some embodiments, the known C5 antagonist is selected from the group consisting of MB12/22, MB12/22-RGD, ARC187, ARC1905, SSL7, and OmCI.

In some embodiments, binding is determined by surface plasmon resonance. In some embodiments, binding is determined by biolayer interferometry. In some embodiments, binding is determined by mass spectrometry. In some embodiments, binding is determined by an immunoassay. In some embodiments, the immunoassay is an enzyme-linked immunosorbent assay (ELISA). In some embodiments, the immunoassay is a radioimmunoassay (RIA).

In some embodiments, the test compound is selected from: an antibody, a small molecule, a polypeptide, a polypeptide analog, a peptidomimetic, a nucleic acid, a nucleic acid analog, and an aptamer. In some embodiments, the test compound is present in a library. In some embodiments, the test compound is rationally designed to bind the wild-type C5 polypeptide. In some embodiments, the test compound is rationally designed to bind a C5 convertase-binding site of C5. In some embodiments, the test compound is rationally designed to bind an epitope of C5 set forth between or comprising residues 872 and 892 of SEQ ID NO:2. In some embodiments, the test compound is designed to bind a C5 convertase-cleavage site of C5. In some embodiments, the test compound is designed to bind to a site on C5 known to be bound by an inhibitor of cleavage of C5.

In certain aspects, the disclosure provides an assay for identifying a potential drug candidate for treating subjects non-responsive to a known C5 antagonist therapy, the assay comprising determining inhibition of a C5 polypeptide-dependent activity of a variant C5 polypeptide using a test compound, wherein inhibition by the test compound is indicative of a potential drug candidate for treating said non-responders.

In some embodiments, the subject has a C5-associated disorder is selected from the group comprising paroxysmal nocturnal hemoglobinuria (PNH), atypical hemolytic-uremic syndrome (aHUS), shiga toxin E. coli-related hemolytic uremic syndrome (STEC-HUS), dense deposit disease (DDD), C3 nephropathy, myasthenia gravis, neuromyelitis optica, cold agglutinin disease (CAD), antineutrophil cytoplasm antibody (ANCA)-associated vasculitis (AAV), asthma, age-related macular degeneration (AMD), transplant rejection, Goodpasture's syndrome, glomerulonephritis, vasculitis, rheumatoid arthritis, dermatitis, systemic lupus erythematosus (SLE), Guillain-Barr syndrome (GBS), dermatomyositis, psoriasis, Graves' disease, Hashimoto's thyroiditis, type I diabetes, pemphigus, autoimmune hemolytic anemia (AIHA), idiopathic thrombocytopenic purpura (ITP), lupus nephritis, ischemia-reperfusion injury, thrombotic thrombocytopenic purpura (TTP), Pauci-immune vasculitis, epidermolysis bullosa, multiple sclerosis, spontaneous fetal loss, recurrent fetal loss, traumatic brain injury, injury resulting from myocardial infarction, cardiopulmonary bypass and hemodialysis, and hemolysis, elevated liver enzymes, and low platelets (HELLP) syndrome. In some embodiments, the subject has PNH. In some embodiments, the subject has aHUS.

In some embodiments, the variant C5 polypeptide is obtained from a subject non-responsive to treatment with a known C5 antagonist.

In some embodiments, the known C5 antagonist is an anti-C5 antibody or an antigen binding fragment thereof, a small molecule, a polypeptide, a polypeptide analog, a peptidomimetic, or an aptamer. In some embodiments, the known C5 antagonist is eculizumab. In some embodiments, the known C5 antagonist is pexelizumab. In some embodiments, the known C5 antagonist is selected from the group consisting of MB12/22, MB12/22-RGD, ARC187, ARC1905, SSL7, and OmCI.

In some embodiments, the test compound is selected from: an antibody, a small molecule, a polypeptide, a polypeptide analog, a peptidomimetic, a nucleic acid, a nucleic acid analog, and an aptamer. In some embodiments, the test compound is present in a library. In some embodiments, the test compound is rationally designed to bind the wild-type C5 polypeptide. In some embodiments, the test compound is rationally designed to bind a C5 convertase-binding site of C5. In some embodiments, the test compound is rationally designed to bind an epitope of C5 set forth between or comprising residues 872 and 892 of SEQ ID NO:2 or 47, e.g., an epitope comprising at least five (e.g., at least six, seven, eight, nine, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 125, 150, 175, 200, 250, 300, 350, 400, 450, 500, 550, 600, 650, 700, 750, 800, or 850) consecutive amino acids of SEQ ID NO:2 or 47, inclusive of amino acid 885. In some embodiments, the test compound is designed to bind a C5 convertase-cleavage site of C5. In some embodiments, the test compound is designed to bind to a site on C5 known to be bound by an inhibitor of cleavage of C5.

In some embodiments, the C5 polypeptide-dependent activity comprises complement-mediated hemolysis. In some embodiments, hemolysis is mediated by the classical complement pathway. In some embodiments, the hemolysis is mediated by the alternative complement pathway. In some embodiments, complement-mediated hemolysis is measured using a hemolytic assay. In some embodiments, the hemolytic assay comprises reconstitution of C5-depleted serum with the C5-polypeptide.

In some embodiments, the C5 polypeptide-dependent activity comprises generation of biologically active products of C5. In some embodiments, the C5 polypeptide-dependent activity comprises generation of C5a and/or C5b. In some embodiments, C5 or biologically active fragments thereof are detected by an immunoassay. In some embodiments, the immunoassay is an enzyme-linked immunosorbent assay (ELISA). In some embodiments, C5 or biologically active fragments thereof are detected by immunoblotting.

In some embodiments, the C5 polypeptide-dependent activity is generation of C5b-9. In some embodiments, C5b-9 formation is assayed using an immunoassay. In some embodiments, C5b-9 formation is assayed using a CH50eq assay.

In certain aspects, the disclosure provides an assay for identifying compounds that potentiate or inhibit cleavage of C5 into C5a and C5b, the assay comprising (i) forming a first reaction mixture comprising: (a) a C5 polypeptide, (b) a C5 convertase, and (c) a test compound, (ii) allowing the first reaction mixture to react, (iii) detecting the amount of C5a or C5b formed in the first reaction mixture, (iv) forming a second reaction mixture comprising: (a) the C5 polypeptide and (b) the C5 convertase, (v) allowing the second reaction mixture to react, (vi) detecting the amount of C5a or C5b formed in the second reaction mixture, and (vii) comparing the amounts of the C5a or C5b formed in steps (ii) and (v), wherein if more of the C5a or C5b is formed in step (ii) than in step (v) then the compound potentiates cleavage of C5 and if less of the C5a or C5b is formed in step (ii) than in step (v) then the test compound inhibits cleavage of C5.

In some embodiments, the C5 polypeptide comprises an amino acid sequence set forth in SEQ ID NO:2 or a fragment thereof. In some embodiments, the C5 polypeptide is obtained from a subject non-responsive to treatment with a known C5 antagonist.

In some embodiments, the known C5 antagonist is an anti-C5 antibody or an antigen binding fragment thereof, a small molecule, a polypeptide, a polypeptide analog, a peptidomimetic, or an aptamer. In some embodiments, the known C5 antagonist is eculizumab. In some embodiments, the known C5 antagonist is pexelizumab. In some embodiments, the known C5 antagonist is selected from the group consisting of MB12/22, MB12/22-RGD, ARC187, ARC1905, SSL7, and OmCI.

In some embodiments, the reaction mixture comprises a cellular preparation. In some embodiments, the reaction mixture is a cell-free polypeptide preparation.

In some embodiments, the test compound is selected from: an antibody, a small molecule, a polypeptide, a polypeptide analog, a peptidomimetic, a nucleic acid, a nucleic acid analog, and an aptamer. In some embodiments, the test compound is present in a library. In some embodiments, the test compound is rationally designed to bind the wild-type C5 polypeptide. In some embodiments, the test compound is rationally designed to bind a C5 convertase-binding site of C5. In some embodiments, the test compound is rationally designed to bind an epitope of C5 set forth between or comprising residues 872 and 892 of SEQ ID NO:2 or 47, e.g., an epitope comprising at least five (e.g., at least six, seven, eight, nine, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 125, 150, 175, 200, 250, 300, 350, 400, 450, 500, 550, 600, 650, 700, 750, 800, or 850) consecutive amino acids of SEQ ID NO:2 or 47, inclusive of amino acid 885. In some embodiments, the test compound is designed to bind a C5 convertase-cleavage site of C5. In some embodiments, the test compound is designed to bind to a site on C5 known to be bound by an inhibitor of cleavage of C5.

In certain aspects, the disclosure provides a method of identifying compounds that inhibit cleavage of C5 into C5a and C5b, the method comprising (i) determining inhibition by a test compound of a wild-type C5 polypeptide activity, and (ii) determining inhibition by the test compound of a variant C5 polypeptide activity, wherein inhibition by the test compound of the wild-type and variant C5 polypeptide activities is indicative of a compound that inhibits cleavage of both wild-type and variant C5 polypeptides.

In some embodiments, the wild-type C5 polypeptide comprises an amino acid sequence set forth in SEQ ID NO:2 or a fragment thereof.

In some embodiments, the variant C5 polypeptide comprises a deletion, an insertion, or a substitution. In some embodiments, the deletion, insertion, or substitution is at a C5 convertase-binding site. In some embodiments, the deletion, insertion, or substitution is present between or inclusive of residues 872 and 892 of SEQ ID NO:2, e.g., the variant polypeptide can comprise or consist of at least five (e.g., at least six, seven, eight, nine, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 125, 150, 175, 200, 250, 300, 350, 400, 450, 500, 550, 600, 650, 700, 750, 800, or 850) consecutive amino acids of SEQ ID NO:47, inclusive of amino acid 885. In some embodiments, the deletion, insertion, or substitution is present at the eculizumab-binding epitope. In some embodiments, the deletion, insertion, or substitution is present at a binding site of a known inhibitor of cleavage of C5 into C5a and C5b. In some embodiments, the variant C5 polypeptide is present in subjects non-responsive to treatment with a known C5 antagonist.

In some embodiments, the known C5 antagonist is an anti-C5 antibody or an antigen binding fragment thereof, a small molecule, a polypeptide, a polypeptide analog, a peptidomimetic, or an aptamer. In some embodiments, the known C5 antagonist is eculizumab. In some embodiments, the known C5 antagonist is pexelizumab. In some embodiments, the known C5 antagonist is selected from the group consisting of MB12/22, MB12/22-RGD, ARC187, ARC1905, SSL7, and OmCI.

In some embodiments, the cleavage of C5 into C5a and C5b is measured using a hemolytic assay. In some embodiments, the hemolytic assay measures classical complement pathway activity. In some embodiments, the hemolytic assay measures alternative complement pathway activity. In some embodiments, the hemolytic assay comprises reconstitution of C5-depleted serum with the C5-polypeptide. In some embodiments, C5, C5a, or C5b is detected by an immunoassay. In some embodiments, the immunoassay is an enzyme-linked immunosorbent assay (ELISA). In some embodiments, C5, C5a, or C5b is detected by immunoblotting. In some embodiments, the cleavage of C5 into C5a and C5b is determined by assaying for the formation of C5b-9. In some embodiments, C5b-9 formation is assayed using an immunoassay. In some embodiments, C5b-9 formation is assayed using a CH50eq assay.

In some embodiments, the test compound is selected from: an antibody, a small molecule, a polypeptide, a polypeptide analog, a peptidomimetic, a nucleic acid, a nucleic acid analog, and an aptamer. In some embodiments, the test compound is present in a library. In some embodiments, the test compound is rationally designed to bind the wild-type C5 polypeptide. In some embodiments, the test compound is rationally designed to bind a C5 convertase-binding site of C5. In some embodiments, the test compound is rationally designed to bind an epitope of C5 set forth between or comprising residues 872 and 892 of SEQ ID NO:2 or 47, e.g., an epitope comprising at least five (e.g., at least six, seven, eight, nine, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 125, 150, 175, 200, 250, 300, 350, 400, 450, 500, 550, 600, 650, 700, 750, 800, or 850) amino acids of SEQ ID NO:2 or 47, inclusive of amino acid 885. In some embodiments, the test compound is designed to bind a C5 convertase-cleavage site of C5. In some embodiments, the test compound is designed to bind to a site on C5 known to be bound by an inhibitor of cleavage of C5.

In certain aspects, the disclosure provides an assay for identifying a potential drug candidate for treating subjects non-responsive to a known C5 antagonist therapy, the assay comprising (i) determining binding affinity of a test compound to a wild-type C5 polypeptide, and (ii) determining binding affinity of a test compound to a variant C5 polypeptide, wherein a test compound that binds to the variant C5 polypeptide with greater affinity than the wild-type C5 polypeptide is a potential drug candidate for treating subjects non-responsive to the known C5 antagonist therapy.

In some embodiments, the method further comprises testing the test compound in a complement-mediated hemolysis assay to determine if it inhibits complement mediated hemolysis wherein a test compound that further inhibits complement-mediated hemolysis is identified as a compound that inhibits cleavage of C5 into C5a and C5b.

In some embodiments, the wild-type C5 polypeptide comprises an amino acid sequence set forth in SEQ ID NO:2 or a fragment thereof.

In some embodiments, the variant C5 polypeptide comprises a deletion, an insertion, or a substitution. In some embodiments, the deletion, insertion, or substitution is at a C5 convertase-binding site. In some embodiments, the deletion, insertion, or substitution is present between or inclusive of residues 872 and 892 of SEQ ID NO:2, e.g., the variant polypeptide can comprise or consist of at least five (e.g., at least six, seven, eight, nine, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 125, 150, 175, 200, 250, 300, 350, 400, 450, 500, 550, 600, 650, 700, 750, 800, or 850) consecutive amino acids of SEQ ID NO:47 inclusive of amino acid 885. In some embodiments, the deletion, insertion, or substitution is present at the eculizumab-binding epitope. In some embodiments, the variant C5 polypeptide is present in subjects non-responsive to treatment with a known C5 antagonist.

In some embodiments, the known C5 antagonist is an anti-C5 antibody or an antigen binding fragment thereof, a small molecule, a polypeptide, a polypeptide analog, a peptidomimetic, or an aptamer. In some embodiments, the known C5 antagonist is eculizumab. In some embodiments, the known C5 antagonist is pexelizumab. In some embodiments, the known C5 antagonist is selected from the group consisting of MB12/22, MB12/22-RGD, ARC187, ARC1905, SSL7, and OmCI.

In some embodiments, the binding affinity is determined by surface plasmon resonance. In some embodiments, the binding affinity is determined by biolayer interferometry. In some embodiments, the binding affinity is determined by mass spectrometry. In some embodiments, the binding affinity is determined by an immunoassay. In some embodiments, the immunoassay is an enzyme-linked immunosorbent assay (ELISA). In some embodiments, the immunoassay is a radioimmunoassay (RIA).

In some embodiments, the test compound is selected from: an antibody, a small molecule, a polypeptide, a polypeptide analog, a peptidomimetic, a nucleic acid, a nucleic acid analog, and an aptamer. In some embodiments, the test compound is present in a library. In some embodiments, the test compound is rationally designed to bind the wild-type C5 polypeptide. In some embodiments, the test compound is rationally designed to bind a C5 convertase-binding site of C5. In some embodiments, the test compound is rationally designed to bind an epitope of C5 set forth between or comprising residues 872 and 892 of SEQ ID NO:2. In some embodiments, the test compound is designed to bind a C5 convertase-cleavage site of C5. In some embodiments, the test compound is designed to bind to a site on C5 known to be bound by an inhibitor of cleavage of C5.

In certain aspects, the disclosure provides a method of identifying a compound that binds a wild-type C5 polypeptide at a region within or overlapping with the region of the wild-type C5 polypeptide bound by a known wild-type C5 antagonist, the method comprising (i) providing a variant C5 polypeptide to which a known wild-type C5 antagonist compound (a) does not bind or (b) binds with lower affinity as compared to the affinity of the known wild-type C5 antagonist for a wild-type C5 polypeptide, (ii) determining whether a test compound binds to the variant C5 polypeptide, and (iii) determining whether the test compound binds to the wild-type C5 polypeptide, wherein a test compound that binds to the wild-type C5 polypeptide, but not to the variant C5 polypeptide or a test compound that preferentially binds to a wild-type C5 polypeptide as compared to the variant C5 polypeptide, is indicative of a compound that binds to the wild-type C5 polypeptide at a region within or overlapping with the region of the wild-type C5 polypeptide bound by the known wild-type C5 antagonist.

In some embodiments, the test compound inhibits cleavage of C5 into fragments C5a and C5b. In some embodiments, the method further comprises determining whether the test compound inhibits the cleavage of C5 into fragments C5a and C5b.

In some embodiments, the wild-type C5 polypeptide comprises an amino acid sequence set forth in SEQ ID NO:2 or a fragment thereof.

In some embodiments, the variant C5 polypeptide comprises a deletion, an insertion, or a substitution. In some embodiments, the deletion, insertion, or substitution is at a C5 convertase-binding site. In some embodiments, the deletion, insertion, or substitution is present between or inclusive of residues 872 and 892 of SEQ ID NO:2, e.g., the variant polypeptide can comprise or consist of at least five (e.g., at least six, seven, eight, nine, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 125, 150, 175, 200, 250, 300, 350, 400, 450, 500, 550, 600, 650, 700, 750, 800, or 850) consecutive amino acids of SEQ ID NO:47 inclusive of amino acid 885. In some embodiments, the deletion, insertion, or substitution is present at the eculizumab-binding epitope. In some embodiments, the variant C5 polypeptide is present in subjects non-responsive to treatment with a known C5 antagonist.

In some embodiments, the known wild-type C5 antagonist is an anti-C5 antibody or an antigen binding fragment thereof, a small molecule, a polypeptide, a polypeptide analog, a peptidomimetic, or an aptamer. In some embodiments, the known wild-type C5 antagonist is eculizumab. In some embodiments, the known wild-type C5 antagonist is pexelizumab. In some embodiments, the known wild-type C5 antagonist is selected from the group consisting of MB12/22, MB12/22-RGD, ARC187, ARC1905, SSL7, and OmCI.

In some embodiments, the determining of whether the test compound binds to the variant C5 polypeptide or the wild-type polypeptide is performed by surface plasmon resonance, biolayer interferometry, or mass spectrometry. In some embodiments, the determining of whether the test compound binds to the variant C5 polypeptide or the wild-type polypeptide is performed using an immunoassay. In some embodiments, the immunoassay is an enzyme-linked immunosorbent assay (ELISA) or a radioimmunoassay (RIA).

In some embodiments, the determining of whether the test compound binds to the variant C5 polypeptide comprises determining the binding affinity of the test compound for the variant C5 polypeptide. In some embodiments, the determining whether the test compound binds to the wild-type C5 polypeptide comprises determining the binding affinity of the test compound for the wild-type C5 polypeptide. In some embodiments, the binding affinity is determined by surface plasmon resonance, biolayer interferometry, or mass spectrometry.

In some embodiments, the test compound is selected from: an antibody, a small molecule, a polypeptide, a polypeptide analog, a peptidomimetic, a nucleic acid, a nucleic acid analog, and an aptamer. In some embodiments, the test compound is present in a library. In some embodiments, the test compound is rationally designed to bind the wild-type C5 polypeptide. In some embodiments, the test compound is rationally designed to bind a C5 convertase-binding site of C5. In some embodiments, the test compound is rationally designed to bind an epitope of C5 set forth between or comprising residues 872 and 892 of SEQ ID NO:2 or 47, e.g., an epitope comprising at least five (e.g., at least six, seven, eight, nine, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 125, 150, 175, 200, 250, 300, 350, 400, 450, 500, 550, 600, 650, 700, 750, 800, or 850) consecutive amino acids of SEQ ID NO:2 or 47, inclusive of amino acid 885. In some embodiments, the test compound is designed to bind a C5 convertase-cleavage site of C5. In some embodiments, the test compound is designed to bind to a site on C5 known to be bound by an inhibitor of cleavage of C5.

In certain aspects, the disclosure provides a method of identifying a compound that binds to a variant C5 polypeptide at a region within or overlapping with the region of the wild-type C5 polypeptide bound by a known wild-type C5 antagonist, the method comprising (i) providing a variant C5 polypeptide to which a known wild-type C5 antagonist compound: (a) does not bind or (b) binds with lower affinity as compared to the affinity of the known wild-type C5 antagonist for a wild-type C5 polypeptide, (ii) determining whether a test compound binds to the variant C5 polypeptide, and (iii) determining whether the test compound binds to the wild-type C5 polypeptide, wherein a test compound that binds to the variant C5 polypeptide, but not to the wild-type C5 polypeptide or a test compound that preferentially binds to a variant C5 polypeptide as compared to the wild-type C5 polypeptide, is indicative of a compound that binds to the variant C5 polypeptide at a region within or overlapping with the region of the wild-type C5 polypeptide bound by the known wild-type C5 antagonist.

In some embodiments, the wild-type C5 polypeptide comprises an amino acid sequence set forth in SEQ ID NO:2 or a fragment thereof.

In some embodiments, the variant C5 polypeptide comprises a deletion, an insertion, or a substitution. In some embodiments, the deletion, insertion, or substitution is at a C5 convertase-binding site. In some embodiments, the deletion, insertion, or substitution is present between or inclusive of residues 872 and 892 of SEQ ID NO:2, e.g., the variant polypeptide can comprise or consist of at least five (e.g., at least six, seven, eight, nine, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 125, 150, 175, 200, 250, 300, 350, 400, 450, 500, 550, 600, 650, 700, 750, 800, or 850) consecutive amino acids of SEQ ID NO:47 inclusive of amino acid 885. In some embodiments, the deletion, insertion, or substitution is present at the eculizumab-binding epitope. In some embodiments, the variant C5 polypeptide is present in subjects non-responsive to treatment with a known C5 antagonist.

In some embodiments, the known C5 antagonist is an anti-C5 antibody or an antigen binding fragment thereof, a small molecule, a polypeptide, a polypeptide analog, a peptidomimetic, or an aptamer. In some embodiments, the known C5 antagonist is eculizumab. In some embodiments, the known C5 antagonist is pexelizumab. In some embodiments, the known C5 antagonist is selected from the group consisting of MB12/22, MB12/22-RGD, ARC187, ARC1905, SSL7, and OmCI.

In some embodiments, the binding affinity is determined by surface plasmon resonance. In some embodiments, the binding affinity is determined by biolayer interferometry. In some embodiments, the binding affinity is determined by mass spectrometry. In some embodiments, the binding affinity is determined by an immunoassay. In some embodiments, the immunoassay is an enzyme-linked immunosorbent assay (ELISA). In some embodiments, the immunoassay is a radioimmunoassay (RIA).

In some embodiments, the test compound is selected from: an antibody, a small molecule, a polypeptide, a polypeptide analog, a peptidomimetic, a nucleic acid, a nucleic acid analog, and an aptamer. In some embodiments, the test compound is present in a library. In some embodiments, the test compound is rationally designed to bind the wild-type C5 polypeptide. In some embodiments, the test compound is rationally designed to bind a C5 convertase-binding site of C5. In some embodiments, the test compound is rationally designed to bind an epitope of C5 set forth between or comprising residues 872 and 892 of SEQ ID NO:2 or 47, e.g., an epitope comprising at least five (e.g., at least six, seven, eight, nine, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 125, 150, 175, 200, 250, 300, 350, 400, 450, 500, 550, 600, 650, 700, 750, 800, or 850) consecutive amino acids of SEQ ID NO:2 or 47, inclusive of amino acid 885. In some embodiments, the test compound is designed to bind a C5 convertase-cleavage site of C5. In some embodiments, the test compound is designed to bind to a site on C5 known to be bound by an inhibitor of cleavage of C5.

In certain aspects, the disclosure provides a method of identifying a compound that binds to a wild-type C5 polypeptide at a region within or overlapping with the region of the wild-type C5 polypeptide bound by a known wild-type C5 antagonist, the method comprising (i) providing a variant C5 polypeptide to which the known wild-type C5 antagonist compound (a) does not bind or (b) binds with lower affinity as compared to the affinity of the known wild-type C5 antagonist for wild-type C5 polypeptide, (ii) determining the binding affinity of a test compound to the variant C5 polypeptide, (iii) determining the binding affinity of the test compound to the wild-type C5 polypeptide, and (iv) comparing the binding affinity from step (ii) to the binding affinity from step (iii), wherein greater affinity of the test compound for the wild-type C5 polypeptide, as compared to the affinity of the test compound for the variant C5 polypeptide, is indicative of a compound that binds to the wild-type C5 polypeptide at a region within or overlapping with the region of the wild-type C5 polypeptide bound by the known wild-type C5 antagonist.

In some embodiments, the wild-type C5 polypeptide comprises an amino acid sequence set forth in SEQ ID NO:2 or a fragment thereof.

In some embodiments, the variant C5 polypeptide comprises a deletion, an insertion, or a substitution. In some embodiments, the deletion, insertion, or substitution is at a C5 convertase-binding site. In some embodiments, the deletion, insertion, or substitution is present between or inclusive of residues 872 and 892 of SEQ ID NO:2, e.g., the variant polypeptide can comprise or consist of at least five (e.g., at least six, seven, eight, nine, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 125, 150, 175, 200, 250, 300, 350, 400, 450, 500, 550, 600, 650, 700, 750, 800, or 850) consecutive amino acids of SEQ ID NO:47 inclusive of amino acid 885. In some embodiments, the deletion, insertion, or substitution is present at the eculizumab-binding epitope. In some embodiments, the variant C5 polypeptide is present in subjects non-responsive to treatment with a known C5 antagonist.

In some embodiments, the known C5 antagonist is an anti-C5 antibody or an antigen binding fragment thereof, a small molecule, a polypeptide, a polypeptide analog, a peptidomimetic, or an aptamer. In some embodiments, the known C5 antagonist is eculizumab. In some embodiments, the known C5 antagonist is pexelizumab. In some embodiments, the known C5 antagonist is selected from the group consisting of MB12/22, MB12/22-RGD, ARC187, ARC1905, SSL7, and OmCI.

In some embodiments, the binding affinity is determined by surface plasmon resonance. In some embodiments, the binding affinity is determined by biolayer interferometry. In some embodiments, the binding affinity is determined by mass spectrometry. In some embodiments, the binding affinity is determined by an immunoassay. In some embodiments, the immunoassay is an enzyme-linked immunosorbent assay (ELISA). In some embodiments, the immunoassay is a radioimmunoassay (RIA).

In some embodiments, the test compound is selected from: an antibody, a small molecule, a polypeptide, a polypeptide analog, a peptidomimetic, a nucleic acid, a nucleic acid analog, and an aptamer. In some embodiments, the test compound is present in a library. In some embodiments, the test compound is rationally designed to bind the wild-type C5 polypeptide. In some embodiments, the test compound is rationally designed to bind a C5 convertase-binding site of C5. In some embodiments, the test compound is rationally designed to bind an epitope of C5 set forth between or comprising residues 872 and 892 of SEQ ID NO:2 or 47, e.g., an epitope comprising at least five (e.g., at least six, seven, eight, nine, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 125, 150, 175, 200, 250, 300, 350, 400, 450, 500, 550, 600, 650, 700, 750, 800, or 850) consecutive amino acids of SEQ ID NO:2 or 47, inclusive of amino acid 885. In some embodiments, the test compound is designed to bind a C5 convertase-cleavage site of C5. In some embodiments, the test compound is designed to bind to a site on C5 known to be bound by an inhibitor of cleavage of C5.

In certain aspects, the disclosure provides a method of identifying a compound that binds to a variant C5 polypeptide at a region within or overlapping with the region of the wild-type polypeptide bound by a known wild-type antagonist, the method comprising (i) providing a variant C5 polypeptide to which the known wild-type C5 antagonist compound (a) does not bind or (b) binds with lower affinity as compared to the affinity of the known wild-type C5 antagonist for wild-type C5 polypeptide, (ii) determining the binding affinity of a test compound to the variant C5 polypeptide, (iii) determining the binding affinity of the test compound to the wild-type C5 polypeptide, and (iv) comparing the binding affinity from step (ii) to the binding affinity from step (iii), wherein greater affinity of the test compound for the variant C5 polypeptide, as compared to the affinity of the test compound for the wild-type C5 polypeptide, is indicative of a compound that binds to the variant C5 polypeptide at a region within or overlapping with the region of the wild-type C5 polypeptide bound by the known wild-type C5 antagonist.

In some embodiments, the wild-type C5 polypeptide comprises an amino acid sequence set forth in SEQ ID NO:2 or a fragment thereof.

In some embodiments, the variant C5 polypeptide comprises a deletion, an insertion, or a substitution. In some embodiments, the deletion, insertion, or substitution is at a C5 convertase-binding site. In some embodiments, the deletion, insertion, or substitution is present between or inclusive of residues 872 and 892 of SEQ ID NO:2, e.g., the variant polypeptide can comprise or consist of at least five (e.g., at least six, seven, eight, nine, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 125, 150, 175, 200, 250, 300, 350, 400, 450, 500, 550, 600, 650, 700, 750, 800, or 850) consecutive amino acids of SEQ ID NO:47 inclusive of amino acid 885. In some embodiments, the deletion, insertion, or substitution is present at the eculizumab-binding epitope. In some embodiments, the variant C5 polypeptide is present in subjects non-responsive to treatment with a known C5 antagonist.

In some embodiments, the known C5 antagonist is an anti-C5 antibody or an antigen binding fragment thereof, a small molecule, a polypeptide, a polypeptide analog, a peptidomimetic, or an aptamer. In some embodiments, the known C5 antagonist is eculizumab. In some embodiments, the known C5 antagonist is pexelizumab. In some embodiments, the known C5 antagonist is selected from the group consisting of MB12/22, MB12/22-RGD, ARC187, ARC1905, SSL7, and OmCI.

In some embodiments, the binding affinity is determined by surface plasmon resonance. In some embodiments, the binding affinity is determined by biolayer interferometry. In some embodiments, the binding affinity is determined by mass spectrometry. In some embodiments, the binding affinity is determined by an immunoassay. In some embodiments, the immunoassay is an enzyme-linked immunosorbent assay (ELISA). In some embodiments, the immunoassay is a radioimmunoassay (RIA).

In some embodiments, the test compound is selected from: an antibody, a small molecule, a polypeptide, a polypeptide analog, a peptidomimetic, a nucleic acid, a nucleic acid analog, and an aptamer. In some embodiments, the test compound is present in a library. In some embodiments, the test compound is rationally designed to bind the wild-type C5 polypeptide. In some embodiments, the test compound is rationally designed to bind a C5 convertase-binding site of C5. In some embodiments, the test compound is rationally designed to bind an epitope of C5 set forth between or comprising residues 872 and 892 of SEQ ID NO:2 or 47, e.g., an epitope comprising at least five (e.g., at least six, seven, eight, nine, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 125, 150, 175, 200, 250, 300, 350, 400, 450, 500, 550, 600, 650, 700, 750, 800, or 850) consecutive amino acids of SEQ ID NO:2 or 47, inclusive of amino acid 885. In some embodiments, the test compound is designed to bind a C5 convertase-cleavage site of C5. In some embodiments, the test compound is designed to bind to a site on C5 known to be bound by an inhibitor of cleavage of C5.

In certain aspects, the disclosure provides a method of selecting a compound that binds to a wild-type C5 polypeptide and a variant C5 polypeptide, the method comprising (i) providing a variant C5 polypeptide to which a known wild-type C5 antagonist compound: (a) does not bind or (b) binds with lower affinity as compared to the affinity of the known wild-type C5 antagonist for a wild-type C5 polypeptide, (ii) determining whether a test compound binds to the variant C5 polypeptide, (iii) determining whether the test compound binds to the wild-type C5 polypeptide; and (v) selecting the test compound if it binds to the wild-type C5 polypeptide and the variant C5 polypeptide.

In some embodiments, the wild-type C5 polypeptide comprises an amino acid sequence set forth in SEQ ID NO:2 or a fragment thereof.

In some embodiments, the variant C5 polypeptide comprises a deletion, an insertion, or a substitution. In some embodiments, the deletion, insertion, or substitution is at a C5 convertase-binding site. In some embodiments, the deletion, insertion, or substitution is present between or inclusive of residues 872 and 892 of SEQ ID NO:2, e.g., the variant polypeptide can comprise or consist of at least five (e.g., at least six, seven, eight, nine, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 125, 150, 175, 200, 250, 300, 350, 400, 450, 500, 550, 600, 650, 700, 750, 800, or 850) consecutive amino acids of SEQ ID NO:47 inclusive of amino acid 885. In some embodiments, the deletion, insertion, or substitution is present at the eculizumab-binding epitope. In some embodiments, the variant C5 polypeptide is present in subjects non-responsive to treatment with a known C5 antagonist.

In some embodiments, the known C5 antagonist is an anti-C5 antibody or an antigen binding fragment thereof, a small molecule, a polypeptide, a polypeptide analog, a peptidomimetic, or an aptamer. In some embodiments, the known C5 antagonist is eculizumab. In some embodiments, the known C5 antagonist is pexelizumab. In some embodiments, the known C5 antagonist is selected from the group consisting of MB12/22, MB12/22-RGD, ARC187, ARC1905, SSL7, and OmCI.

In some embodiments, the binding affinity is determined by surface plasmon resonance. In some embodiments, the binding affinity is determined by biolayer interferometry. In some embodiments, the binding affinity is determined by mass spectrometry. In some embodiments, the binding affinity is determined by an immunoassay. In some embodiments, the immunoassay is an enzyme-linked immunosorbent assay (ELISA). In some embodiments, the immunoassay is a radioimmunoassay (RIA).

In some embodiments, the test compound is selected from: an antibody, a small molecule, a polypeptide, a polypeptide analog, a peptidomimetic, a nucleic acid, a nucleic acid analog, and an aptamer. In some embodiments, the test compound is present in a library. In some embodiments, the test compound is rationally designed to bind the wild-type C5 polypeptide. In some embodiments, the test compound is rationally designed to bind a C5 convertase-binding site of C5. In some embodiments, the test compound is rationally designed to bind an epitope of C5 set forth between or comprising residues 872 and 892 of SEQ ID NO:2 or 47, e.g., an epitope comprising at least five (e.g., at least six, seven, eight, nine, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 125, 150, 175, 200, 250, 300, 350, 400, 450, 500, 550, 600, 650, 700, 750, 800, or 850) consecutive amino acids of SEQ ID NO:2 or 47, inclusive of amino acid 885. In some embodiments, the test compound is designed to bind a C5 convertase-cleavage site of C5. In some embodiments, the test compound is designed to bind to a site on C5 known to be bound by an inhibitor of cleavage of C5.

In certain aspects, the disclosure provides a method of screening for a compound that binds to a wild-type C5 polypeptide and a variant C5 polypeptide, the method comprising (i) providing a variant C5 polypeptide to which a known wild-type C5 antagonist compound: (a) does not bind or (b) binds with lower affinity as compared to the affinity of the known wild-type C5 antagonist for a wild-type C5 polypeptide, (ii) providing a library of test compounds, (iii) screening a plurality of the test compounds for binding to the variant C5 polypeptide, (iv) screening a plurality of the test compounds for binding to the wild-type C5 polypeptide, and (v) selecting one or more test compounds that bind to the wild-type C5 polypeptide and the variant C5 polypeptide.

In some embodiments, the wild-type C5 polypeptide comprises an amino acid sequence set forth in SEQ ID NO:2 or a fragment thereof.

In some embodiments, the variant C5 polypeptide comprises a deletion, an insertion, or a substitution. In some embodiments, the deletion, insertion, or substitution is at a C5 convertase-binding site. In some embodiments, the deletion, insertion, or substitution is present between or inclusive of residues 872 and 892 of SEQ ID NO:2, e.g., the variant polypeptide can comprise or consist of at least five (e.g., at least six, seven, eight, nine, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 125, 150, 175, 200, 250, 300, 350, 400, 450, 500, 550, 600, 650, 700, 750, 800, or 850) consecutive amino acids of SEQ ID NO:47 inclusive of amino acid 885. In some embodiments, the deletion, insertion, or substitution is present at the eculizumab-binding epitope. In some embodiments, the variant C5 polypeptide is present in subjects non-responsive to treatment with a known C5 antagonist.

In some embodiments, the known C5 antagonist is an anti-C5 antibody or an antigen binding fragment thereof, a small molecule, a polypeptide, a polypeptide analog, a peptidomimetic, or an aptamer. In some embodiments, the known C5 antagonist is eculizumab. In some embodiments, the known C5 antagonist is pexelizumab. In some embodiments, the known C5 antagonist is selected from the group consisting of MB12/22, MB12/22-RGD, ARC187, ARC1905, SSL7, and OmCI.

In some embodiments, the binding affinity is determined by surface plasmon resonance. In some embodiments, the binding affinity is determined by biolayer interferometry. In some embodiments, the binding affinity is determined by mass spectrometry. In some embodiments, the binding affinity is determined by an immunoassay. In some embodiments, the immunoassay is an enzyme-linked immunosorbent assay (ELISA). In some embodiments, the immunoassay is a radioimmunoassay (RIA).

In some embodiments of any of the methods described herein, the test compound is selected from: an antibody, a small molecule, a polypeptide, a polypeptide analog, a peptidomimetic, a nucleic acid, a nucleic acid analog, and an aptamer. In some embodiments, the test compound is present in a library. In some embodiments, the test compound is rationally designed to bind the wild-type C5 polypeptide. In some embodiments, the test compound is rationally designed to bind a C5 convertase-binding site of C5. In some embodiments, the test compound is rationally designed to bind an epitope of C5 set forth between or comprising residues 872 and 892 of SEQ ID NO:2 or 47, e.g., an epitope comprising at least five (e.g., at least six, seven, eight, nine, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 125, 150, 175, 200, 250, 300, 350, 400, 450, 500, 550, 600, 650, 700, 750, 800, or 850) consecutive amino acids of SEQ ID NO:2 or 47, inclusive of amino acid 885. In some embodiments, the test compound is designed to bind a C5 convertase-cleavage site of C5. In some embodiments, the test compound is designed to bind to a site on C5 known to be bound by an inhibitor of cleavage of C5.

In certain aspects, the disclosure provides a method of screening for a compound that preferentially binds to a wild-type C5 polypeptide as compared to the binding of the compound to a variant C5 polypeptide, the method comprising (i) providing a variant C5 polypeptide to which a known wild-type C5 antagonist compound: (a) does not bind or (b) binds with lower affinity as compared to the affinity of the known wild-type C5 antagonist for a wild-type C5 polypeptide, (ii) providing a library of test compounds, (iii) screening a plurality of the test compounds for binding to the wild-type C5 polypeptide to identify test compounds that bind to the wild-type C5 polypeptide, (iv) screening one or more of the test compounds identified in (iii) for binding to the variant C5 polypeptide, and (v) selecting at least one test compound that binds to the wild-type C5 polypeptide but does not bind to the variant C5 polypeptide or preferentially binds to the wild-type C5 polypeptide as compared to the binding of the test compound to the variant C5 polypeptide.

In some embodiments, the wild-type C5 polypeptide comprises an amino acid sequence set forth in SEQ ID NO:2 or a fragment thereof.

In some embodiments, the variant C5 polypeptide comprises a deletion, an insertion, or a substitution. In some embodiments, the deletion, insertion, or substitution is at a C5 convertase-binding site. In some embodiments, the deletion, insertion, or substitution is present between or inclusive of residues 872 and 892 of SEQ ID NO:2, e.g., the variant polypeptide can comprise or consist of at least five (e.g., at least six, seven, eight, nine, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 125, 150, 175, 200, 250, 300, 350, 400, 450, 500, 550, 600, 650, 700, 750, 800, or 850) consecutive amino acids of SEQ ID NO:47 inclusive of amino acid 885. In some embodiments, the deletion, insertion, or substitution is present at the eculizumab-binding epitope. In some embodiments, the variant C5 polypeptide is present in subjects non-responsive to treatment with a known C5 antagonist.

In some embodiments, the known C5 antagonist is an anti-C5 antibody or an antigen binding fragment thereof, a small molecule, a polypeptide, a polypeptide analog, a peptidomimetic, or an aptamer. In some embodiments, the known C5 antagonist is eculizumab. In some embodiments, the known C5 antagonist is pexelizumab. In some embodiments, the known C5 antagonist is selected from the group consisting of MB12/22, MB12/22-RGD, ARC187, ARC1905, SSL7, and OmCI.

In some embodiments, the binding affinity is determined by surface plasmon resonance. In some embodiments, the binding affinity is determined by biolayer interferometry. In some embodiments, the binding affinity is determined by mass spectrometry. In some embodiments, the binding affinity is determined by an immunoassay. In some embodiments, the immunoassay is an enzyme-linked immunosorbent assay (ELISA). In some embodiments, the immunoassay is a radioimmunoassay (RIA).

In some embodiments, the test compound is selected from: an antibody, a small molecule, a polypeptide, a polypeptide analog, a peptidomimetic, a nucleic acid, a nucleic acid analog, and an aptamer. In some embodiments, the test compound is present in a library. In some embodiments, the test compound is rationally designed to bind the wild-type C5 polypeptide. In some embodiments, the test compound is rationally designed to bind a C5 convertase-binding site of C5. In some embodiments, the test compound is rationally designed to bind an epitope of C5 set forth between or comprising residues 872 and 892 of SEQ ID NO:2 or 47, e.g., an epitope comprising at least five (e.g., at least six, seven, eight, nine, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 125, 150, 175, 200, 250, 300, 350, 400, 450, 500, 550, 600, 650, 700, 750, 800, or 850) consecutive amino acids of SEQ ID NO:2 or 47, inclusive of amino acid 885. In some embodiments, the test compound is designed to bind a C5 convertase-cleavage site of C5. In some embodiments, the test compound is designed to bind to a site on C5 known to be bound by an inhibitor of cleavage of C5.

In some embodiments, the disclosure provides a method of screening for a compound that preferentially binds to a variant C5 polypeptide as compared to the binding of the compound to a wild-type C5 polypeptide, the method comprising (i) providing a variant C5 polypeptide to which a known wild-type C5 antagonist compound: (a) does not bind or (b) binds with lower affinity as compared to the affinity of the known wild-type C5 antagonist for a wild-type C5 polypeptide, (ii) providing a library of test compounds, (iii) screening a plurality of the test compounds for binding to the variant C5 polypeptide to identify test compounds that bind to the variant C5 polypeptide, (iv) screening one or more of the test compounds identified in (iii) for binding to the wild-type C5 polypeptide, and (v) selecting at least one test compound that binds to the variant C5 polypeptide but does not bind to the wild-type C5 polypeptide or preferentially binds to the variant C5 polypeptide as compared to the binding of the test compound to the wild-type C5 polypeptide.

In some embodiments, the wild-type C5 polypeptide comprises an amino acid sequence set forth in SEQ ID NO:2 or a fragment thereof.

In some embodiments, the variant C5 polypeptide comprises a deletion, an insertion, or a substitution. In some embodiments, the deletion, insertion, or substitution is at a C5 convertase-binding site. In some embodiments, the deletion, insertion, or substitution is present between or inclusive of residues 872 and 892 of SEQ ID NO:2. In some embodiments, the deletion, insertion, or substitution is present at the eculizumab-binding epitope. In some embodiments, the variant C5 polypeptide is present in subjects non-responsive to treatment with a known C5 antagonist.

In some embodiments, the known C5 antagonist is an anti-C5 antibody or an antigen binding fragment thereof, a small molecule, a polypeptide, a polypeptide analog, a peptidomimetic, or an aptamer. In some embodiments, the known C5 antagonist is eculizumab. In some embodiments, the known C5 antagonist is pexelizumab. In some embodiments, the known C5 antagonist is selected from the group consisting of MB12/22, MB12/22-RGD, ARC187, ARC1905, SSL7, and OmCI.

In some embodiments, the binding affinity is determined by surface plasmon resonance. In some embodiments, the binding affinity is determined by biolayer interferometry. In some embodiments, the binding affinity is determined by mass spectrometry. In some embodiments, the binding affinity is determined by an immunoassay. In some embodiments, the immunoassay is an enzyme-linked immunosorbent assay (ELISA). In some embodiments, the immunoassay is a radioimmunoassay (RIA).

In some embodiments, the test compound is selected from: an antibody, a small molecule, a polypeptide, a polypeptide analog, a peptidomimetic, a nucleic acid, a nucleic acid analog, and an aptamer. In some embodiments, the test compound is present in a library. In some embodiments, the test compound is rationally designed to bind the wild-type C5 polypeptide. In some embodiments, the test compound is rationally designed to bind a C5 convertase-binding site of C5. In some embodiments, the test compound is rationally designed to bind an epitope of C5 set forth between or comprising residues 872 and 892 of SEQ ID NO:2 or 47, e.g., an epitope comprising at least five (e.g., at least six, seven, eight, nine, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 125, 150, 175, 200, 250, 300, 350, 400, 450, 500, 550, 600, 650, 700, 750, 800, or 850) consecutive amino acids of SEQ ID NO:2 or 47, inclusive of amino acid 885. In some embodiments, the test compound is designed to bind a C5 convertase-cleavage site of C5. In some embodiments, the test compound is designed to bind to a site on C5 known to be bound by an inhibitor of cleavage of C5.

In certain aspects, the disclosure provides a method of screening for a compound that binds to a wild-type C5 polypeptide and a variant C5 polypeptide, the method comprising (i) providing a variant C5 polypeptide to which a known wild-type C5 antagonist compound: (a) does not bind or (b) binds with lower affinity as compared to the affinity of the known wild-type C5 antagonist for a wild-type C5 polypeptide, (ii) providing a library of test compounds, (iii) screening a plurality of the test compounds for binding to the wild-type C5 polypeptide to identify test compounds that bind to the wild-type C5 polypeptide, (iv) screening one or more of the test compounds identified in (iii) for binding to the variant C5 polypeptide, and (v) selecting at least one test compound that binds to the wild-type C5 polypeptide and binds to the variant C5 polypeptide.

In some embodiments, the wild-type C5 polypeptide comprises an amino acid sequence set forth in SEQ ID NO:2 or a fragment thereof.

In some embodiments, the variant C5 polypeptide comprises a deletion, an insertion, or a substitution. In some embodiments, the deletion, insertion, or substitution is at a C5 convertase-binding site. In some embodiments, the deletion, insertion, or substitution is present between or inclusive of residues 872 and 892 of SEQ ID NO:2, e.g., the variant polypeptide can comprise or consist of at least five (e.g., at least six, seven, eight, nine, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 125, 150, 175, 200, 250, 300, 350, 400, 450, 500, 550, 600, 650, 700, 750, 800, or 850) consecutive amino acids of SEQ ID NO:47 inclusive of amino acid 885. In some embodiments, the deletion, insertion, or substitution is present at the eculizumab-binding epitope. In some embodiments, the variant C5 polypeptide is present in subjects non-responsive to treatment with a known C5 antagonist.

In some embodiments, the known C5 antagonist is an anti-C5 antibody or an antigen binding fragment thereof, a small molecule, a polypeptide, a polypeptide analog, a peptidomimetic, or an aptamer. In some embodiments, the known C5 antagonist is eculizumab. In some embodiments, the known C5 antagonist is pexelizumab. In some embodiments, the known C5 antagonist is selected from the group consisting of MB12/22, MB12/22-RGD, ARC187, ARC1905, SSL7, and OmCI.

In some embodiments, the binding affinity is determined by surface plasmon resonance. In some embodiments, the binding affinity is determined by biolayer interferometry. In some embodiments, the binding affinity is determined by mass spectrometry. In some embodiments, the binding affinity is determined by an immunoassay. In some embodiments, the immunoassay is an enzyme-linked immunosorbent assay (ELISA). In some embodiments, the immunoassay is a radioimmunoassay (RIA).

In some embodiments, the test compound is selected from: an antibody, a small molecule, a polypeptide, a polypeptide analog, a peptidomimetic, a nucleic acid, a nucleic acid analog, and an aptamer. In some embodiments, the test compound is present in a library. In some embodiments, the test compound is rationally designed to bind the wild-type C5 polypeptide. In some embodiments, the test compound is rationally designed to bind a C5 convertase-binding site of C5. In some embodiments, the test compound is rationally designed to bind an epitope of C5 set forth between or comprising residues 872 and 892 of SEQ ID NO:2 or 47, e.g., an epitope comprising at least five (e.g., at least six, seven, eight, nine, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 125, 150, 175, 200, 250, 300, 350, 400, 450, 500, 550, 600, 650, 700, 750, 800, or 850) consecutive amino acids of SEQ ID NO:2 or 47, inclusive of amino acid 885. In some embodiments, the test compound is designed to bind a C5 convertase-cleavage site of C5. In some embodiments, the test compound is designed to bind to a site on C5 known to be bound by an inhibitor of cleavage of C5.

One of skill in the art will appreciate that the above-described methods can be used in conjunction with known agonists or antagonists of variant polypeptides, in a manner similar to that described above for known agonists or antagonists of wild-type polypeptides. For example, relying on information about known agonists or antagonists of variant polypeptides, the methods can be used to identify compounds that bind to the corresponding wild-type polypeptide at a region within or overlapping with the region of a variant form of the wild-type polypeptide bound by a known agonist or antagonist of the variant polypeptide. Such a method can comprise: (i) providing a variant polypeptide to which a known agonist or antagonist compound binds; (ii) providing the wild-type form of the variant polypeptide to which the known agonist or antagonist: (a) does not bind or (b) binds with lower affinity as compared to the affinity of the known agonist or antagonist for the variant polypeptide; (iii) determining whether a test compound binds to the variant polypeptide; and (iv) determining whether the test compound binds to the wild-type polypeptide; wherein a test compound that binds to the wild-type polypeptide, but not to the variant polypeptide, or a test compound that preferentially binds to the wild-type polypeptide as compared to the variant polypeptide is indicative of a compound that binds to the wild-type polypeptide at a region within or overlapping with the region of the variant polypeptide bound by the known agonist or antagonist.

In another aspect, the disclosure features a method of identifying a compound that binds to a variant form of a wild-type polypeptide at a region within or overlapping with the region of the variant form of the polypeptide bound by a known agonist or antagonist of the variant form of the wild-type polypeptide, which method includes: (i) providing a variant polypeptide to which a known agonist or antagonist compound binds; (ii) providing the wild-type form of the variant polypeptide to which the known agonist or antagonist: (a) does not bind or (b) binds with lower affinity as compared to the affinity of the known agonist or antagonist for the variant polypeptide; (iii) determining whether a test compound binds to the variant polypeptide; and (iv) determining whether the test compound binds to the wild-type polypeptide; wherein a test compound that binds to the variant polypeptide, but not to the wild-type polypeptide, or a test compound that preferentially binds to the variant polypeptide as compared to the wild-type polypeptide is indicative of a compound that binds to the variant polypeptide at a region within or overlapping with the region of the variant polypeptide bound by the known agonist or antagonist.

In some embodiments, any of the above methods can further include the step of selecting a test compound that binds to the variant polypeptide, but not to the wild-type polypeptide, or a test compound that preferentially binds to the variant polypeptide as compared to the wild-type polypeptide. In some embodiments, any of the above methods can further include the step of selecting a test compound that binds to the wild-type polypeptide, but not to the variant polypeptide, or a test compound that preferentially binds to the wild-type polypeptide as compared to the variant polypeptide.

In yet another aspect, the disclosure features a method of screening for a compound that binds to a wild-type polypeptide at a region within or overlapping with the region of a variant form of the wild-type polypeptide bound by a known agonist or antagonist of the variant form of the wild-type polypeptide. This method comprises: (i) providing the variant form of a wild-type polypeptide to which a known agonist or antagonist compound binds; (ii) providing the wild-type polypeptide to which the agonist or antagonist: (a) does not bind or (b) binds with lower affinity as compared to the affinity of the known agonist or antagonist for the variant form of the polypeptide; (iii) providing a library of test compounds; (iv) screening a plurality of the test compounds for binding to the variant polypeptide; (v) screening a plurality of the test compounds for binding to the wild-type polypeptide; and (vi) selecting one or more test compounds that bind to the wild-type polypeptide, but not to the variant polypeptide or that preferentially bind to the wild-type polypeptide as compared to the variant polypeptide, wherein such compounds are indicative of compounds that bind to the wild-type polypeptide at a region within or overlapping with the region of the variant polypeptide bound by the known agonist or antagonist.

Furthermore, in another aspect, the disclosure features a method of screening for a compound that binds to a variant form of a wild-type polypeptide at a region within or overlapping with the region of the variant polypeptide bound by a known agonist or antagonist of the variant polypeptide. The method can include: (i) providing a variant polypeptide to which a known agonist or antagonist compound binds; (ii) providing the wild-type form of the variant polypeptide to which the known agonist or antagonist: (a) does not bind or (b) binds with lower affinity as compared to the affinity of the known agonist or antagonist for the variant polypeptide; (iii) providing a library of test compounds; (iv) screening a plurality of the test compounds for binding to the variant polypeptide; (v) screening a plurality of the test compounds for binding to the wild-type polypeptide; and (vi) selecting one or more test compounds that bind to the variant polypeptide, but not to the wild-type polypeptide or that preferentially bind to the variant polypeptide as compared to the wild-type polypeptide, wherein such compounds are indicative of compounds that bind to the variant polypeptide at a region within or overlapping with the region of the variant polypeptide bound by the known agonist or antagonist.

For use in such methods, suitable wild-type and variant polypeptides and test compounds include, but are not limited to, any of those described herein.

Unless otherwise defined, all technical and scientific terms used herein have the same meaning as commonly understood by one of ordinary skill in the art to which this disclosure pertains. In case of conflict, the present document, including definitions, will control. Preferred methods and materials are described below, although methods and materials similar or equivalent to those described herein can also be used in the practice or testing of the presently disclosed methods and compositions. All publications, patent applications, patents, and other references mentioned herein are incorporated by reference in their entirety.

Other features and advantages of the present disclosure, e.g., screening methods, will be apparent from the following description, the examples, and from the claims.

### BRIEF DESCRIPTION OF THE DRAWINGS

FIG. 1 shows the amino acid sequence for human C5 (SEQ ID NO:2). One portion of the conformational epitope bound by eculizumab is indicated by a box and is the sequence of SEQ ID NO:4.

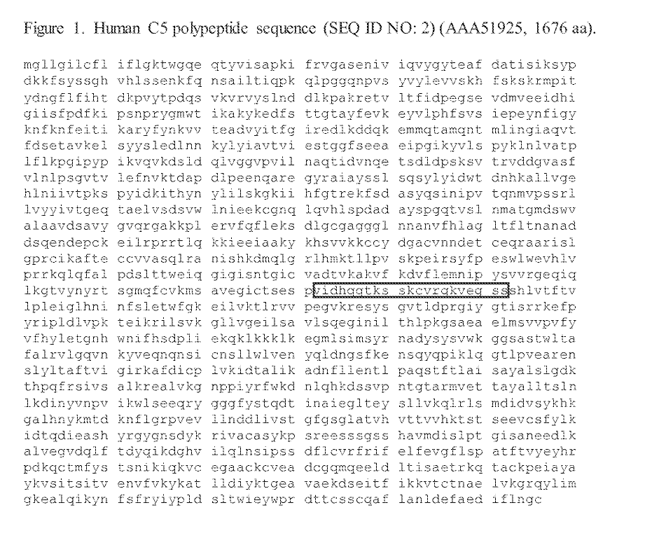

FIG. 2 shows a three-dimensional structure of human C5. The epitope bound by eculizumab is indicated on the structure.

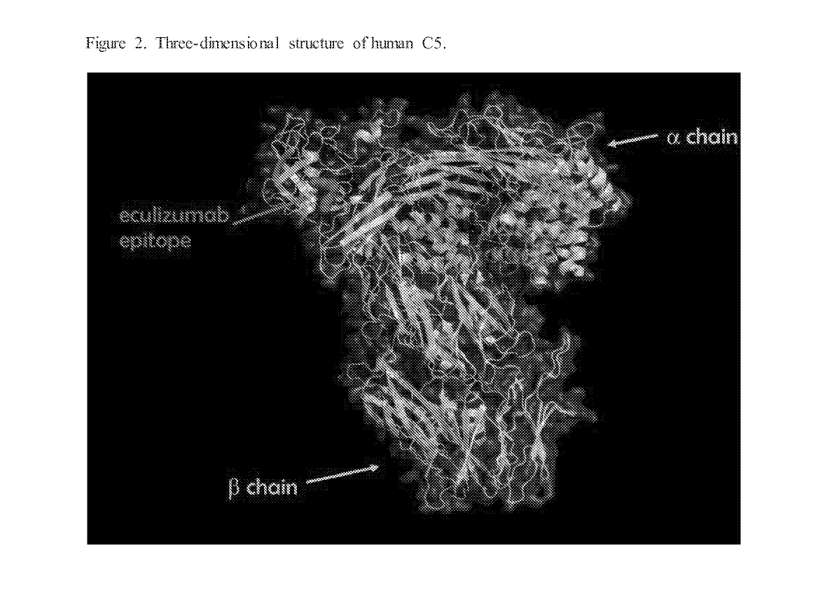

### DETAILED DESCRIPTION

The present disclosure provides compositions, kits and methods for screening for novel compounds that bind to polypeptides of therapeutic interest (e.g., polypeptides implicated in, or known to contribute to, the pathogenesis of human disease). These compounds can, in some embodiments, bind to a polypeptide of therapeutic interest at a region that is bound by a known agonist or antagonist of the polypeptide. In some embodiments, these compounds bind to components of the complement cascade and, in some embodiments, such compounds inhibit complement-mediated activity. The present disclosure also provides kits and methods for predicting the responsiveness of a subject to treatment with a complement inhibitor.

### Overview of the Complement System

The complement system acts in conjunction with other immunological systems of the body to defend against intrusion of cellular and viral pathogens. There are at least 25 complement proteins, which are found as a complex collection of plasma proteins and membrane cofactors. The plasma proteins make up about 10% of the globulins in vertebrate serum. Complement components achieve their immune defensive functions by interacting in a series of intricate but precise enzymatic cleavage and membrane binding events. The resulting complement cascade leads to the production of products with opsonic, immunoregulatory, and lytic functions. A concise summary of the biologic activities associated with complement activation is provided, for example, in The Merck Manual, 16^{th} Edition.

The complement cascade progresses via the classical pathway, the alternative pathway, or the lectin pathway. These pathways share many components, and while they differ in their initial steps, they converge and share the same “terminal complement” components (C5 through C9) responsible for the activation and destruction of target cells.

The classical pathway (CP) is typically initiated by antibody recognition of, and binding to, an antigenic site on a target cell. The alternative pathway (AP) can be antibody independent, and can be initiated by certain molecules on pathogen surfaces. Additionally, the lectin pathway is typically initiated with binding of mannose-binding lectin (MBL) to high mannose substrates. These pathways converge at the point where complement component C3 is cleaved by an active protease to yield C3a and C3b. Other pathways activating complement attack can act later in the sequence of events leading to various aspects of complement function. C3a is an anaphylatoxin. C3b binds to bacterial and other cells, as well as to certain viruses and immune complexes, and tags them for removal from the circulation. This opsonic function of C3b is generally considered to be the most important anti-infective action of the complement system. C3b also forms a complex with other components unique to each pathway to form classical or alternative C5 convertase, which cleaves complement component C5 (hereinafter referred to as “C5”) into C5a and C5b.

Cleavage of C5 releases biologically active species such as for example C5a, a potent anaphylatoxin and chemotactic factor, and C5b which through a series of protein interactions leads to the formation of the lytic terminal complement complex, C5b-9. C5a and C5b-9 also have pleiotropic cell activating properties, by amplifying the release of downstream inflammatory factors, such as hydrolytic enzymes, reactive oxygen species, arachidonic acid metabolites and various cytokines.

C5b combines with C6, C7, and C8 to form the C5b-8 complex at the surface of the target cell. Upon binding of several C9 molecules, the membrane attack complex (MAC, C5b-9, terminal complement complex—TCC) is formed. When sufficient numbers of MACs insert into target cell membranes the openings they create (MAC pores) mediate rapid osmotic lysis of the target cells. Lower, non-lytic concentrations of MACs can produce other effects. In particular, membrane insertion of small numbers of the C5b-9 complexes into endothelial cells and platelets can cause deleterious cell activation. In some cases activation may precede cell lysis.

As mentioned above, C3a and C5a, activated complement components, can trigger mast cell degranulation, which releases histamine from basophils and mast cells, and other mediators of inflammation, resulting in smooth muscle contraction, increased vascular permeability, leukocyte activation, and other inflammatory phenomena including cellular proliferation resulting in hypercellularity. C5a also functions as a chemotactic peptide that serves to attract pro-inflammatory granulocytes to the site of complement activation. C5a receptors are found on the surfaces of bronchial and alveolar epithelial cells and bronchial smooth muscle cells. C5a receptors have also been found on eosinophils, mast cells, monocytes, neutrophils, and activated lymphocytes.

Specific modulators, e.g., inhibitors of complement component C5 that do not block the functions of early complement components will not substantially impair the opsonization functions associated with C3b and are particularly useful as therapeutic agents in the treatment of disorders characterized by the deleterious effects of complement activation.

As discussed above, in many cases a given drug, such as an inhibitor of complement component C5 may have little or no effect on a sub-section of the population. Genetic variation often underlies the lack of or poor response to the inhibitors of C5. In fact, it has been observed that certain sections of the populations suffering from C5-associated disorders show poor or lack of responsiveness when treated with known C5 antagonists. While this disclosure is not bound by any particular theory or mechanism of action, the inventors believe that genetic variation in the C5 component is the likely cause of such non-responsiveness. For example, the non-responders may have a mutation(s), such as an insertion, deletion, or substitution, in the C5 gene which results in modifications and/or alterations in the binding pocket, i.e., in the amino acid sequence of the region of the C5 component involved in interacting with or binding to the C5 antagonist(s). This could potentially alter the binding/binding affinity of the C5 antagonist(s) such that the C5 antagonist(s) do not effectively bind and/or inhibit the expression and/or activities of the mutant or variant C5 polypeptide or do not inhibit the cleavage of C5. The present disclosure provides methods of using a wild-type C5 polypeptide and/or a variant C5 polypeptide to screen for novel compounds that can inhibit C5 activity and/or its conversion into biologically active products. The present disclosure also provides methods for predicting the responsiveness of a subject to treatment with a C5 antagonist.

### DEFINITIONS

For convenience, certain terms employed in the specification, examples, and appended claims are collected here.

As used herein, the term “complement inhibitor” refers to any agent that interacts with, inhibits, or downregulates an activity of the complement cascade. One of skill in the art would appreciate that complete inhibition is not required. For example, it is sufficient that the inhibitor has an IC₅₀ of less than 1 μM in a hemolysis assay.

As used herein, the term “C5 antagonist” refers to any agent that inhibits the cleavage of a human C5 protein into C5a and C5b.

The complement inhibitor can be in the form of a pharmaceutically acceptable salt, free-base, solvate, hydrate, stereoisomer, clathrate or prodrug thereof. Such inhibitory activity can be determined by an assay or animal model well known in the art including those set forth in greater detail herein.

As used herein, the term “conversion of C5” refers to the conversion of C5 into the biologically active species C5a and C5b as a result of cleavage of C5.

As used herein, the term “C5 convertase” can refer to either the classical pathway C5 convertase C3bC4bC2a or the alternative pathway convertase (C3b)₂Bb.

As used herein, the term “C5 convertase-binding site” refers to any protein determinant on the surface of the C5 polypeptide that is involved in the recognition and/or binding of C5 convertases.

As used herein, the term “C5 convertase-cleavage site” refers to the proteolytic cleavage site located between Arg733 and Leu734 of the wild-type C5 polypeptide (SEQ ID NO:3), which is identical to residues 751 and 752 of SEQ ID NO:2.

As used herein, the term “eculizumab-binding epitope” refers to the region of the C5 polypeptide that is capable of specific binding to eculizumab. The epitope is a conformational epitope, one portion of which is contained within the sequence set forth in SEQ ID NO:4 and comprises at least the KSSKC (SEQ ID NO:46) peptide (residues 861-865 of the wild-type C5 polypeptide (SEQ ID NO:3), which are identical to residues 879-883 of SEQ ID NO:2.

As used herein, the term “C5-associated disorder” refers to any condition characterized by C5-mediated complement dysfunction such as, but not limited to, paroxysmal nocturnal hemoglobinuria (PNH), atypical hemolytic-uremic syndrome (aHUS), shiga toxin E. coli-related hemolytic uremic syndrome (STEC-HUS), dense deposit disease (DDD), C3 nephropathy, myasthenia gravis, neuromyelitis optica, cold agglutinin disease (CAD), antineutrophil cytoplasm antibody (ANCA)-associated vasculitis (AAV), asthma, age-related macular degeneration (AMD), transplant rejection, Goodpasture's syndrome, glomerulonephritis, vasculitis, rheumatoid arthritis, dermatitis, systemic lupus erythematosus (SLE), Guillain-Barr syndrome (GBS), dermatomyositis, psoriasis, Graves' disease, Hashimoto's thyroiditis, type I diabetes, pemphigus, autoimmune hemolytic anemia (AIHA), idiopathic thrombocytopenic purpura (ITP), lupus nephritis, ischemia-reperfusion injury, thrombotic thrombocytopenic purpura (TTP), Pauci-immune vasculitis, epidermolysis bullosa, multiple sclerosis, spontaneous fetal loss, recurrent fetal loss, traumatic brain injury, injury resulting from myocardial infarction, cardiopulmonary bypass and hemodialysis, and hemolysis, elevated liver enzymes, and low platelets (HELLP) syndrome.

As used herein, the term “non-responder” refers to any subject showing little or no response to treatment with a known C5 antagonist or any subject who is identified as carrying a mutation that is linked to non-responsiveness to treatment with a C5 antagonist.

As used herein, the term “rational drug design” refers to a method of drug design wherein biologically active compounds are designed based on the three-dimensional structure of the target or are designed based on known modulators of the target.

### Complement Component C5 and Variants Thereof

C5 is a 190 kDa beta globulin found in normal serum at a concentration of approximately 75 μg/mL (0.4 μM). C5 is glycosylated, with about 1.5 to 3 percent of its mass attributed to carbohydrates. Mature C5 is a heterodimer of a 999 amino acid 115 kDa alpha chain that is disulfide linked to a 655 amino acid 75 kDa beta chain. C5 is synthesized as a single chain precursor protein product of a single copy gene (Haviland et al. (1991) J Immunol 146:362-368). The C5 gene comprises 41 exons, the corresponding nucleotide sequences of which are listed in Table 1. The cDNA sequence of the transcript of this gene, set forth in SEQ ID NO:1, predicts a secreted pro-C5 precursor of 1658 amino acids along with an 18 amino acid leader sequence (see, e.g., U.S. Pat. No. 6,355,245) as set forth in SEQ ID NO:2. The amino acid sequence of the pro-C5 precursor is set forth in SEQ ID NO:3.

The pro-C5 precursor (SEQ ID NO:3) is cleaved after amino acids 655 and 659, to yield the beta chain as an amino terminal fragment (amino acid residues +1 to 655 of the above sequence) and the alpha chain as a carboxyl terminal fragment (amino acid residues 660 to 1658 of the above sequence), with four amino acids (amino acid residues 656-659 of the above sequence) deleted between the two.

C5a is cleaved from the alpha chain of C5 by either alternative or classical C5 convertase as an amino terminal fragment comprising the first 74 amino acids of the alpha chain (i.e., amino acid residues 660-733 of SEQ ID NO:3). Approximately 20 percent of the 11 kDa mass of C5a is attributed to carbohydrate. The cleavage site for convertase action is at, or immediately adjacent to, amino acid residue 733 of SEQ ID NO:3.

C5 can also be activated by means other than C5 convertase activity. Limited trypsin digestion (see, e.g., Minta and Man (1997) J Immunol 119:1597-1602 and Wetsel and Kolb (1982) J Immunol 128:2209-2216), thrombin, and acid treatment (Yamamoto and Gewurz (1978) J Immunol 120:2008 and Damerau et al. (1989) Molec Immunol 26:1133-1142) can also cleave C5 and produce active C5b.

A compound that would bind C5 at, or adjacent to, the cleavage site for convertase action (located between amino residues 733 and 734 of SEQ ID NO:3) would have the potential to block access of the C5 convertase enzymes to the cleavage site and thereby act as a complement inhibitor. A compound that would bind at, or adjacent to, any of the sites that recognize and/or bind the C5 convertase enzymes would also block the interaction of C5 with the C5 convertase enzymes and thereby act as a complement inhibitor.

The use of a C5 antagonist, such as eculizumab (Soliris®; Alexion Pharmaceuticals, Inc., Cheshire, Conn.) (see, e.g., Kaplan (2002) Curr Opin Investig Drugs 3(7):1017-23; Hill (2005) Clin Adv Hematol Oncol 3(11):849-50; and Rother et al. (2007) Nature Biotechnology 25(11):1256-1488), an antibody which inhibits conversion of C5 into the biologically active species C5a and C5b, by binding to C5 with high affinity and blocking the binding of C5 to the C5 convertases, has been approved for the treatment of patients with paroxysmal nocturnal hemoglobinuria (PNH) and atypical hemolytic uremic syndrome (aHUS). PNH is a progressive and life-threatening disease characterized by the excessive destruction of red blood cells (hemolysis). aHUS is an ultra-rare, life-threatening genetic disease that can progressively damage vital organs, leading to stroke, heart attack, kidney failure and death. While treatment with eculizumab has been very successful, a small number of patients have been identified to be non-responsive to treatment with eculizumab.

Variations in the nucleotide sequence of the C5 gene (Table 1) can result in modifications at the protein level in or around any of the binding pockets and/or epitopes recognized by the C5 antagonists (e.g., eculizumab) such that a subject carrying such a variant C5 gene would be non-responsive to treatment with a C5 antagonist. One of skill in the art would recognize that such variations can occur in other polypeptides of therapeutic interest and thus render inactive or less effective therapeutic agents (e.g., known agonists or antagonists) that bind to the polypeptides at regions containing the variations. The term “variation” is used interchangeably with the term “mutation”. For example, a mutation in or around the epitope recognized by a C5 antagonist such as eculizumab, which includes the sequence set forth in SEQ ID NO:4, could result in a decreased affinity of the variant C5 polypeptide for eculizumab such that treatment with eculizumab would not effectively inhibit complement-mediated hemolysis. The variations in the nucleotide sequence of C5 can comprise a deletion, an insertion or a substitution. Such variations in the nucleotide sequence, when present in a coding region of the gene, result in corresponding changes in the C5 protein. In one embodiment, the variation is present in the alpha chain of the C5 molecule. In a further embodiment, the variation is present in or around the epitope recognized by eculizumab, for example, within the sequence set forth in SEQ ID NO:4. In another embodiment, the variation is present in the region around the proteolytic cleavage site between residues 733 and 734 of SEQ ID NO:3. For example, the variation is present between residues 727 and 744 of SEQ ID NO:3. In one embodiment, the variation is present in the beta chain of the C5 molecule. The presence of the variation can cause a loss of binding, or decrease in binding affinity, to a C5 antagonist such as, but not limited to, those described above, leading to loss of effective inhibition by the C5 antagonist. The term variant is intended to include a DNA mutant obtained by in vitro mutagenesis of the wild-type DNA (Table 1) according to methods known in the art.

In some embodiments, the variation in C5 is at amino acid position 885 of SEQ ID NO:2, e.g., in which the arginine at position 885 is substituted by histidine (R885H). In some embodiments, the variant C5 polypeptide comprises or consists of the amino acid sequence depicted in SEQ ID NO:47 or 48. In some embodiments, the variant C5 polypeptide comprises at least five (e.g., at least six, seven, eight, nine, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 125, 150, 175, 200, 250, 300, 350, 400, 450, 500, 550, 600, 650, 700, 750, 800, or 850) amino acids of SEQ ID NO:47 inclusive of histidine 885. In some embodiments, the variant C5 polypeptide: (a) comprises at least 20 (e.g., at least 21, 22, 23, 24, 25, 26, 27, 28, 29, 30, 35, 40, 45, 50, 55, 60, 65, 70, 75, 80, 85, 90, 95, 100, 125, 150, 175, 200, 250, 300, 350, 400, 450, 500, 550, 600, 650, 700, 750, 800, or 850) amino acids, (b) is at least 80% identical to a corresponding at least 20 amino acid sequence of SEQ ID NO:47, and (c) comprises histidine 885 of SEQ ID NO:47.

The presence of a mutation, as described above, in the C5 gene can be indicative of non-responsiveness to treatment with a C5 antagonist. The presence or absence of a mutation in the C5 gene can be determined using methods described herein to predict the responsiveness of a subject to treatment with a C5 antagonist.

In some embodiments, any of the methods described herein can include the step of generating a variant form of a wild-type polypeptide, i.e., a variant form of a wild-type polypeptide which no longer binds or binds with lower affinity to a known agonist or antagonist of the wild-type form of the polypeptide. Methods for introducing one or more amino acid substitutions, deletions, or insertions into a wild-type polypeptide are well-known in the art. See, e.g., Sambrook et al. (1989) “Molecular Cloning: A Laboratory Manual, 2^{nd} Edition,” Cold Spring Harbor Laboratory Press, Cold Spring Harbor, N.Y. Methods for monitoring whether a modification of a wild-type polypeptide results in loss of affinity for a known agonist or antagonist of the wild-type protein are also well-known in the art and include, e.g., routine binding assays such as an ELISA, SPR assays, immunoprecipitation assay, affinity chromatography, and equilibrium dialysis as described above. Immunoassays which can be used to analyze immunospecific binding and cross-reactivity of the antibodies include, but are not limited to, competitive and non-competitive assay systems using techniques such as Western blots, radioimmunoassays, ELISA, “sandwich” immunoassays, immunoprecipitation assays, immunodiffusion assays, agglutination assays, complement-fixation assays, immunoradiometric assays, fluorescent immunoassays, and protein A immunoassays. Briefly, a variant form of the wild-type polypeptide can be generated using well-known molecular biology techniques and assayed for its ability to bind to an agonist or antagonist known to bind to the wild-type form of the polypeptide. A loss of binding, or a reduction (e.g., a substantial reduction), in the ability of the known agonist or antagonist to bind to the variant polypeptide, as compared to the wild-type polypeptide, indicates that the one or more substitutions, deletions, or insertions in the variant polypeptide have affected the binding site of the known agonist or antagonist. Such variants are suitable for use in the screening methods described herein.

In some embodiments, the variant polypeptides or portions thereof (e.g., variant C5 polypeptides) can be isolated from populations that are non-responsive to treatment with a known agonist or antagonist, or they can be designed based on binding pockets and/or epitopes that are known to be recognized by existing antagonists or agonists. Full length variant polypeptides or fragments thereof may be used as immunogens to produce antibodies specific to the variant polypeptides.

The nucleic acid(s) encoding a variant polypeptide (or a wild-type polypeptide) can be inserted into an expression vector that comprises transcriptional and translational regulatory sequences, which include, e.g., promoter sequences, ribosomal binding sites, transcriptional start and stop sequences, translational start and stop sequences, transcription terminator signals, polyadenylation signals, and enhancer or activator sequences. The regulatory sequences include a promoter and transcriptional start and stop sequences. In addition, the expression vector can include more than one replication system such that it can be maintained in two different organisms, for example in mammalian or insect cells for expression and in a prokaryotic host for cloning and amplification.

Several possible vector systems are available for the expression of polypeptides from nucleic acids in mammalian cells. One class of vectors relies upon the integration of the desired gene sequences into the host cell genome. Cells which have stably integrated DNA can be selected by simultaneously introducing drug resistance genes such as E. coli gpt (Mulligan and Berg (1981) Proc Natl Acad Sci USA 78:2072) or Tn5 neo (Southern and Berg (1982) Mol Appl Genet 1:327). The selectable marker gene can be either linked to the DNA gene sequences to be expressed, or introduced into the same cell by co-transfection (Wigler et al. (1979) Cell 16:77). A second class of vectors utilizes DNA elements which confer autonomously replicating capabilities to an extrachromosomal plasmid. These vectors can be derived from animal viruses, such as bovine papillomavirus (Sarver et al. (1982) Proc Natl Acad Sci USA, 79:7147), polyoma virus (Deans et al. (1984) Proc Natl Acad Sci USA 81:1292), or SV40 virus (Lusky and Botchan (1981) Nature 293:79).

The expression vectors can be introduced into cells in a manner suitable for subsequent expression of the nucleic acid. The method of introduction is largely dictated by the targeted cell type, discussed below. Exemplary methods include CaPO₄ precipitation, liposome fusion, lipofectin, electroporation, viral infection, dextran-mediated transfection, polybrene-mediated transfection, and direct microinjection.

Appropriate host cells for the expression of variant polypeptides include, e.g., yeast, bacteria, insect, and mammalian cells. Of particular interest are bacteria such as E. coli, fungi such as Saccharomyces cerevisiae and Pichia pastoris, insect cells such as SF9, mammalian cell lines (e.g., human cell lines), as well as primary cell lines. The type of host cell selected for expression of a polypeptide will depend in part on the particular type of polypeptide to be expressed as well as the intended use of the expressed antibody.

The variant polypeptides (or wild-type polypeptides) are produced from cells by culturing a host cell transformed with the expression vector comprising nucleic acid encoding the polypeptides under conditions, and for an amount of time, sufficient to allow expression of the polypeptides. Such conditions for protein expression will vary with the choice of the expression vector and the host cell, and can be easily ascertained by one skilled in the art through routine experimentation. For example, polypeptides expressed in E. coli can be refolded from inclusion bodies (see, e.g., Hou et al. (1998) Cytokine 10:319-30). Bacterial expression systems and methods for their use are well known in the art. The choice of codons, suitable expression vectors and suitable host cells will vary depending on a number of factors, and may be easily optimized as needed. Variant or wild-type polypeptides can be expressed in mammalian cells or in other expression systems including but not limited to yeast, baculovirus, and in vitro expression systems (see, e.g., Kaszubska et al. (2000) Protein Expression and Purification 18:213-220).

Following expression, the polypeptides can be isolated. The term “isolated” or “purified” as applied to any of the polypeptides described herein refers to a polypeptide that has been separated or purified from components (e.g., proteins or other naturally-occurring biological or organic molecules) which naturally accompany it, e.g., other proteins, lipids, and nucleic acid in a prokaryote expressing the proteins. Typically, a polypeptide is purified when it constitutes at least 60 (e.g., at least 65, 70, 75, 80, 85, 90, 92, 95, 97, or 99) %, by weight, of the total protein in a sample.

The polypeptides can be isolated or purified in a variety of ways known to those skilled in the art depending on what other components are present in the sample. Standard purification methods include electrophoretic, molecular, immunological, and chromatographic techniques, including ion exchange, hydrophobic, affinity, and reverse-phase HPLC chromatography. Ultrafiltration and diafiltration techniques, in conjunction with protein concentration, are also useful. See, e.g., Scopes (1994) “Protein Purification, 3^{rd} edition,” Springer-Verlag, New York City, N.Y. The degree of purification necessary will vary depending on the desired use. In some instances, no purification of the expressed polypeptides will be necessary.

Methods for determining the yield or purity of an isolated polypeptide are known in the art and include, e.g., Bradford assay, UV spectroscopy, Biuret protein assay, Lowry protein assay, amido black protein assay, high pressure liquid chromatography (HPLC), mass spectrometry (MS), and gel electrophoretic methods (e.g., using a protein stain such as Coomassie Blue or colloidal silver stain).

### Modulators of C5 Activity

Inhibitors of human complement component C5 have been described. The terms “inhibitor” and “antagonist” are used interchangeably. As used herein, an “inhibitor of complement component C5” is any agent that inhibits the cleavage of a human C5 protein to forms C5a and C5b.

Exemplary C5 antagonists bind complement component C5 and inhibit complement-mediated activity by inhibiting the conversion of C5 into C5a and C5b. One such exemplary C5 antagonist is eculizumab. This anti-C5 monoclonal antibody recognizes a conformational epitope, one portion of which maps around amino acids 861-865 of the C5 polypeptide (SEQ ID NO:3), corresponding to the peptide KSSKC (SEQ ID NO:46), and is able to inhibit the binding of C5 convertases to C5. Pexelizumab (Alexion Pharmaceuticals, Inc., Cheshire, Conn.) is a single chain variable fragment (scFv) derived from eculizumab (see, e.g., Whiss (2002) Curr Opin Investig Drugs 3(6):870-7; Patel et al. (2005) Drugs Today (Barc) 41(3):165-70; and Thomas et al. (1996) Mol Immunol. 33(17-18):1389-401). Other exemplary antagonists include the anti-C5 minibody MB12/22 (Mubodina®; Adienne Pharma & Biotech, Bergamo, Italy) and a variant form of the minibody fused with RGD peptide, MB12/22-RGD (Ergidina®; Adienne Pharma & Biotech, Bergamo, Italy). MB12/22 and MB12/22-RGD are derived from an anti-C5 scFv, Ts-a12/22, which is described in patent application WO 2004/007553. MB-12/22 and MB-12/22-RGD recognize an epitope comprising the C5 convertase cleavage site located between amino acids 733 and 734 of the C5 polypeptide (SEQ ID NO:3). Other anti-C5 antibodies that variously recognize epitopes on either the alpha chain or the beta chain of the C5 molecule and inhibit complement mediated hemolytic activity, are described in the patent application WO 2010/015608. C5 binding aptamers, ARC187 and ARC1905 (commercially available from Archemix/Ophthotech Corp., Princeton, N.J.), are described in the patent application US 20070048248. OmCI, a protein excreted by the soft tick Ornithodoros moubata, is a naturally occurring inhibitor of C5 activity. A recombinant variant of OmCI, rev576, has also been described (Hepburn et al. (2007) J Biol Chem 282:8292-8299 and Soltys et al. (2009) Ann Neurol 65:67-75). Another naturally occurring inhibitor of C5 activity is the Staphylococcus aureus secreted protein SSL7 (Laursen et al. (2010) PNAS 107:3681-3686).

Other compounds which may be utilized to bind to or otherwise block the activity of complement component C5 include, but are not limited to, proteins, protein fragments, peptides, small molecules, RNA aptamers including L-RNA aptamers, or spiegelmers.

### Screening Methods and Assays

The present disclosure provides methods of using a wild-type C5 polypeptide and/or a variant C5 polypeptide to screen for novel compounds that can inhibit C5 activity and/or conversion into biologically active products. The objective of such a screening strategy is two-fold. One objective is to identify compounds that are more efficacious and/or potent and/or more convenient for dosing (e.g., screening for compounds that can be taken orally) than existing therapies for treating complement-associated disorders and the second objective is to identify compounds that may be used to treat patients who do not respond to existing therapies.

A variety of screening assays are provided below and such assays may be used to identify and evaluate potential C5 antagonists.

In one embodiment, the method of identifying potential C5 antagonists comprises screening for compounds that bind the wild-type C5 polypeptide (SEQ ID NO:3) but do not bind a variant C5 polypeptide. The methods this embodiment will yield compounds that bind wild-type C5 at or around the site of the mutation present in the variant polypeptide, thus enabling the easy identification of agents that target an epitope of interest on the C5 molecule. In a preferred embodiment, the variant C5 polypeptide is one to which a known wild-type C5 antagonist does not bind or binds poorly, i.e., the known antagonist binds to wild-type C5 but binds less well or not at all to the variant C5. Compounds that similarly bind wild-type C5 but do not bind to the variant C5 or bind only poorly to the variant C5 as compared to their binding to wild-type C5 are likely to bind to the same region or an overlapping region of wild-type C5 as does the known C5 antagonist. This is assumed to be true because the known antagonist and the potential antagonist being tested have similar binding characteristics in that they both bind wild-type C5 and bind the same variant of C5 less well than they bind the wild-type C5 or they do not bind at all to the variant C5. Although it may not be true that 100% of compounds selected from such a screening will bind to the same or overlapping region as the known antagonist, further assays can be performed to test this, e.g., competitive binding experiments. This type of screening is especially useful in the case that one has a known antagonist that works extremely well, likely because it binds to an epitope involved in the binding of C5 to a C5 convertase, and one wants to screen for other compounds that bind to that same epitope or overlapping epitopes. For example, if one has a known antagonist that is an antibody and one wants to screen a library of small molecules to find an antagonist that can be administered orally, such a screen will be useful to screen for small molecules that bind to the same epitope or overlapping epitope (region) as does the known antibody antagonist. This will distinguish from merely screening for compounds in the library that bind to C5 but bind to unknown regions, e.g., if one screens merely by testing for which compounds bind to wild-type C5. It would be expected that many such compounds found from merely screening for those that bind to C5, although binding to C5, would not be active in inhibiting C5 from being cleaved into C5a and C5b by C5 convertase because they may bind to epitopes (binding sites) not involved in the binding interaction with a C5 convertase. Adding the step of further screening against a mutated (variant) version of C5 to which the known antagonist does not bind greatly increases the chances that the screening process will yield molecules that bind to the same epitope or overlapping epitope as does the known antagonist since the molecules will have similar binding properties and therefore will increase the chances that the compound will also be an antagonist. This method can be extremely useful when one finds a patient who does not respond to the known antagonist and the patient is found to have a mutation in C5. That mutated C5 can be used in the screening method. Although this particular screen will not find an antagonist that will help the patient with the mutation, it will be useful for screening for compounds as potential antagonists for patients who have the wild-type C5.

In a second embodiment, the method comprises screening for compounds that have a greater binding affinity for the variant C5 polypeptide compared to a wild-type C5 polypeptide, wherein the variant C5 polypeptide is of a sequence found in a patient who does not respond to a known C5 antagonist. A test compound is selected for further characterization if its binding affinity for the variant C5 polypeptide is at least 2-fold, 3-fold, 4-fold, 5-fold, 10-fold, 15-fold, 25-fold, or 50-fold greater than its binding affinity for the wild-type C5 polypeptide. A test compound is selected for further characterization if its binding affinity for the variant C5 polypeptide is at least 2-10 fold greater, 10-15 fold greater, 15-20 fold greater, 20-25 fold greater, 25-30 fold greater, or 30-35 fold greater than its binding affinity for the wild-type C5 polypeptide. The method of the above embodiment will yield compounds that bind to the variant C5 at or around the site of the mutation, thus enabling the easy identification of agents that target a region of inhibitory potential on the variant C5 polypeptide. Such compounds can be used for treating non-responder populations (those who do not respond to the known C5 antagonist) carrying a gene for the variant C5 polypeptide. Methods of determining binding and/or binding affinity of a particular agent to a C5 polypeptide as described herein are well known in the art.

In a third embodiment, the method comprises screening for compounds that bind and/or inhibit the activity of both wild-type and variant C5 polypeptides. A test compound is selected for further characterization if it inhibits the cleaving of C5 into C5a and C5b by at least 70%, at least 60%, at least 50%, at least 40%, at least 30% or at least 25%. A test compound is selected for further characterization if it inhibits the activity of a C5 polypeptide resulting from such cleavage step, including but not limited to fragments C5a or C5b or the C5b-9 terminal complement complex, by 30-40%, 40-50%, 50-60%, 60-70%, 70-80%, 80-90%, or 90-100%. Methods for determining whether a particular agent is an inhibitor of complement component C5 activity as described herein are known in the art.

A variety of assay formats may be used and, in light of the present disclosure, those not expressly described herein will nevertheless be understood by one of ordinary skill in the art. In some embodiments, the disclosure provides cell-free assay systems using purified proteins or protein fragments. Moreover, in place of purified proteins, cellular extracts and lysates may be used to render a suitable cell-free assay system. In some embodiments, the disclosure provides cell-based assay systems. In some embodiments, fragments comprising equivalent portions of the wild-type and variant C5 polypeptides can be used in lieu of full-length polypeptides. An exemplary fragment of the wild-type C5 polypeptide is set forth in SEQ ID NO:4. Additional exemplary fragments of C5 are disclosed in, e.g., U.S. Pat. No. 6,355,245. In some embodiments, a control assay using a known inhibitor of C5 can also be performed to provide a baseline for comparison.

Compounds to be tested can be produced, for example, by bacteria, yeast or other organisms (e.g., natural products), produced chemically (e.g., small molecules, including peptidomimetics), or produced recombinantly. Rational drug design may be employed to design potential C5 antagonists. For example, rational drug design can employ the use of crystal or solution structural information on the human complement component C5 protein. See, e.g., the structures described in Hagemann et al. (2008) J Biol Chem 283(12):7763-75; Zuiderweg et al. (1989) Biochemistry 28(1):172-85 and Laursen et al. (2011) EMBO J 30(3):606-616. Rational drug design can also be achieved based on known compounds, e.g., a known inhibitor of C5 (e.g., an antibody, or antigen-binding fragment thereof, that binds to a human complement component C5 protein). Rational drug design can be further based on the identity and location of mutated residues in variant C5 polypeptides that confer resistance to C5 antagonists. Test compounds contemplated by the present invention include small molecules, polypeptides, polypeptide analogs, peptidomimetics, nucleic acids, nucleic acid analogs, aptamers, and antibodies. In a preferred embodiment, the test agent is a small organic molecule having a molecular weight of less than about 2,500 Daltons.

The test agents can be provided as single, discrete entities, or provided in libraries of greater complexity, such as made by combinatorial chemistry. These libraries can comprise, for example, alcohols, alkyl halides, amines, amides, esters, aldehydes, ethers and other classes of organic compounds. Presentation of test compounds to the test system can be in either an isolated form or as mixtures of compounds, especially in initial screening steps.

In many drug screening programs which test libraries of compounds and natural extracts, high throughput assays are desirable in order to maximize the number of compounds surveyed in a given period of time. Assays that are performed in cell-free systems, such as may be developed with purified or semi-purified proteins or with lysates, are often preferred as “primary” screens in that they can be generated to permit rapid development and relatively easy detection of an alteration in a molecular target which is mediated by a test compound. Moreover, the effects of cellular toxicity and/or bioavailability of the test compound can be generally ignored in the in vitro system, the assay instead being focused primarily on the effect of the drug on the molecular target as may be manifest in an alteration of binding affinity with other proteins or changes in functional properties of the molecular target. These compounds can further be tested in animal models to assess their ability to modulate complement activity in vivo. After identifying an agent using a cell-free system, or any other agent that is expected to modulate complement-mediated activity, the subject test agents can be tested in whole cells or tissues, in vitro or in vivo, to confirm their ability to modulate complement activity. The efficacy of the compound can be assessed by generating dose response curves from data obtained using various concentrations of the test compound. In some embodiments, a control assay can also be performed to provide a baseline for comparison. In the control assay, the activity of the C5 polypeptide is quantitated in the absence of the test compound.

In some embodiments, test compounds identified from these assays may be used in a therapeutic method for treating a C5-associated disorder. C5-associated disorders include, but are not limited to, paroxysmal nocturnal hemoglobinuria (PNH), atypical hemolytic-uremic syndrome (aHUS), shiga toxin E. coli-related hemolytic uremic syndrome (STEC-HUS), dense deposit disease (DDD), C3 nephropathy, myasthenia gravis, neuromyelitis optica, cold agglutinin disease (CAD), antineutrophil cytoplasm antibody (ANCA)-associated vasculitis (AAV), asthma, age-related macular degeneration (AMD), transplant rejection, Goodpasture's syndrome, glomerulonephritis, vasculitis, rheumatoid arthritis, dermatitis, systemic lupus erythematosus (SLE), Guillain-Barr syndrome (GBS), dermatomyositis, psoriasis, Graves' disease, Hashimoto's thyroiditis, type I diabetes, pemphigus, autoimmune hemolytic anemia (AIHA), idiopathic thrombocytopenic purpura (ITP), lupus nephritis, ischemia-reperfusion injury, thrombotic thrombocytopenic purpura (TTP), Pauci-immune vasculitis, epidermolysis bullosa, multiple sclerosis, spontaneous fetal loss, recurrent fetal loss, traumatic brain injury, injury resulting from myocardial infarction, cardiopulmonary bypass and hemodialysis, and hemolysis, elevated liver enzymes, and low platelets (HELLP) syndrome.

### Binding Assays

Methods for determining whether an agent binds to a target protein and/or the affinity for an agent for a target protein are known in the art. For example, the binding of an agent to a target protein can be detected and/or quantified using a variety of techniques such as, but not limited to, BioLayer Interferometry (BLI), Western blot, dot blot, surface plasmon resonance method (SPR), enzyme-linked immunosorbent assay (ELISA), AlphaScreen® or AlphaLISA® assays, or mass spectrometry based methods.

In some embodiments, agents can be assayed using any SPR-based assays known in the art for characterizing the kinetic parameters of the interaction of the agent with C5. Any SPR instrument commercially available including, but not limited to, BIAcore Instruments (Biacore AB; Uppsala, Sweden); 1Asys instruments (Affinity Sensors; Franklin, Mass.); IBIS system (Windsor Scientific Limited; Berks, UK), SPR-CELLIA systems (Nippon Laser and Electronics Lab; Hokkaido, Japan), and SPR Detector Spreeta (Texas Instruments; Dallas, Tex.) can be used in the methods described herein. See, e.g., Mullett et al. (2000) Methods 22: 77-91; Dong et al. (2002) Reviews in Mol Biotech 82: 303-323; Fivash et al. (1998) Curr Opin Biotechnol 9: 97-101; and Rich et al. (2000) Curr Opin Biotechnol 11: 54-61.

In some embodiments, the biomolecular interactions between the agents and C5 can be assayed using BLI on an Octet (ForteBio Inc.). BLI is a label-free optical analytical technique that senses binding between a ligand (such as a C5 polypeptide) that is immobilized on a biosensor tip and an analyte (such as a test compound) in solution by measuring the change in the thickness of the protein layer on the biosensor tip in real-time.

In some embodiments, AlphaScreen (PerkinElmer) assays can be used to characterize binding of test agents to C5. The acronym ALPHA stands for Amplified Luminescent Proximity Homogeneous Assay. AlphaScreen is a bead-based proximity assay that senses binding between molecules (such as a C5 polypeptide and a test compound) attached to donor and acceptor beads by measuring the signal produced by energy transfer between the donor and acceptor beads. (See e.g., Eglen et al. (2008) Curr Chem Genomics 1:2-10).

In some embodiments, AlphaLISA® (PerkinElmer) assays can be used to characterize binding of test agents to C5 polypeptides. AlphaLISA is modified from the AlphaScreen assay described above to include europium-containing acceptor beads and functions as an alternative to traditional ELISA assays. (See, e.g., Eglen et al. (2008) Curr Chem Genomics 1:2-10.)

A variety of immunoassay techniques, including competitive and non-competitive immunoassays, can be used. The term “immunoassay” encompasses techniques including, without limitation, flow cytometry, FACS, enzyme immunoassays (EIA), such as enzyme multiplied immunoassay technique (EMIT), enzyme-linked immunosorbent assay (ELISA), IgM antibody capture ELISA (MAC ELISA) and microparticle enzyme immunoassay (MEIA), furthermore capillary electrophoresis immunoassays (CEIA), radio-immunoassays (RIA), immunoradiometric assays (IRMA), fluorescence polarization immunoassays (FPIA) and chemiluminescence assays (CL). If desired, such immunoassays can be automated. Immunoassays can also be used in conjunction with laser induced fluorescence. Liposome immunoassays, such as flow-injection liposome immunoassays and liposome immunosensors, are also suitable for use in the present invention. In addition, nephelometry assays, in which, for example, the formation of protein/antibody complexes results in increased light scatter that is converted to a peak rate signal as a function of the marker concentration, are suitable for use in the methods of the present invention. In a preferred embodiment of the present invention, the incubation products are detected by ELISA, RIA, fluoro immunoassay (FIA) or soluble particle immune assay (SPIA).

In some embodiments, binding of test agents to C5 polypeptides can be assayed using thermodenaturation methods involving differential scanning fluorimetry (DSF) and differential static light scattering (DSLS).

In some embodiments, binding of test agents to C5 polypeptides can be assayed using a mass spectrometry based method such as, but not limited to, an affinity selection coupled to mass spectrometry (AS-MS) platform. This is a label-free method where the protein and test compound are incubated, unbound molecules are washed away and protein-ligand complexes are analyzed by MS for ligand identification following a decomplexation step.

In some embodiments, binding of test agents to C5 can be quantitated using, for example, detectably labeled proteins such as radiolabeled (e.g., ³²P, ³⁵S, ¹⁴C or ³H), fluorescently labeled (e.g., FITC), or enzymatically labeled C5 polypeptide or test compound, by immunoassay, or by chromatographic detection.

In some embodiments, the present invention contemplates the use of fluorescence polarization assays and fluorescence resonance energy transfer (FRET) assays in measuring, either directly or indirectly, the degree of interaction between a C5 polypeptide and a test compound.

All of the above embodiments are suitable for development into high-throughput platforms. Methods for further characterizing the C5-inhibitory activity of compounds identified using the above methods are described herein and are known in the art.

### Complement Activity Assays

The C5 antagonists described herein can have activity in blocking the generation or activity of the C5a and/or C5b active fragments of a complement component C5 protein (e.g., a human C5 protein). Through this blocking effect, the C5 antagonists inhibit, e.g., the proinflammatory effects of C5a and the generation of the C5b-9 membrane attack complex (MAC) at the surface of a cell.

Methods for determining whether a particular agent is an inhibitor of human complement component C5 as described herein are known in the art Inhibition of human complement component C5 can also reduce the cell-lysing ability of complement in a subject's body fluids. Such reductions of the cell-lysing ability of complement present in the body fluid(s) can be measured by methods well known in the art such as, for example, by a conventional hemolytic assay such as the hemolysis assay described by Kabat and Mayer (eds.), “Experimental Immunochemistry, 2^{nd} Edition,” 135-240, Springfield, Ill., C C Thomas (1961), pages 135-139, or a conventional variation of that assay such as the chicken erythrocyte hemolysis method as described in, e.g., Hillmen et al. (2004) N Engl J Med 350(6):552. Methods for determining whether a candidate compound inhibits the cleavage of human C5 into forms C5a and C5b are known in the art and described in, e.g., Moongkarndi et al. (1982) Immunobiol. 162:397; Moongkarndi et al. (1983) Immunobiol. 165:323; Isenman et al. (1980) J Immunol. 124(1):326-31; Thomas et al. (1996) Mol. Immunol. 33(17-18):1389-401; and Evans et al. (1995) Mol. Immunol. 32(16):1183-95. For example, the concentration and/or physiologic activity of C5a and C5b in a body fluid can be measured by methods well known in the art. Methods for measuring C5a concentration or activity include, e.g., chemotaxis assays, RIAs, or ELISAs (see, e.g., Ward and Zvaifler (1971) J Clin Invest. 50(3):606-16 and Wurzner et al. (1991) Complement Inflamm. 8:328-340). For C5b, hemolytic assays or assays for soluble C5b-9 as discussed herein can be used. Other assays known in the art can also be used. Using assays of these or other suitable types, candidate agents capable of inhibiting human complement component C5 can be screened.

Immunological techniques such as, but not limited to, ELISA can be used to measure the protein concentration of C5 and/or its split products to determine the ability of a test compound to inhibit conversion of C5 into biologically active products. In some embodiments, C5a generation is measured. In some embodiments, C5b-9 neoepitope-specific antibodies are used to detect the formation of terminal complement.

Hemolytic assays can be used to determine the inhibitory activity of a test compound on complement activation. In order to determine the effect of a test compound on classical complement pathway-mediated hemolysis in a serum test solution in vitro, for example, sheep erythrocytes coated with hemolysin or chicken erythrocytes sensitized with anti-chicken erythrocyte antibody are used as target cells. The percentage of lysis is normalized by considering 100% lysis equal to the lysis occurring in the absence of the inhibitor. In some embodiments, the classical complement pathway is activated by a human IgM antibody, for example, as utilized in the Wieslab® Classical Pathway Complement Kit (Wieslab® COMPL CP310, Euro-Diagnostica, Sweden). Briefly, the test serum is incubated with a test compound in the presence of a human IgM antibody. The amount of C5b-9 that is generated is measured by contacting the mixture with an enzyme conjugated anti-C5b-9 antibody and a fluorogenic substrate and measuring the absorbance at the appropriate wavelength. As a control, the test serum is incubated in the absence of the test compound. In some embodiments, the test serum is a C5-deficient serum reconstituted with a C5 polypeptide. In some embodiments, the C5 polypeptide is a variant C5 polypeptide. In some embodiments, the C5 polypeptide is a wild-type C5 polypeptide (SEQ ID NO:3). In some embodiments, the test serum is pooled normal human serum (PNHS). In some embodiments, the test serum is pooled non-responder human serum.

To determine the effect of a test compound on alternative pathway-mediated hemolysis, unsensitized rabbit or guinea pig erythrocytes are used as the target cells. In some embodiments, the serum test solution is a C5-deficient serum reconstituted with a C5 polypeptide. The percentage of lysis is normalized by considering 100% lysis equal to the lysis occurring in the absence of the inhibitor. In some embodiments, the alternative complement pathway is activated by lipopolysaccharide molecules, for example, as utilized in the Wieslab® Alternative Pathway Complement Kit (Wieslab® COMPL AP330, Euro-Diagnostica, Sweden). Briefly, the test serum is incubated with a test compound in the presence of lipopolysaccharide. The amount of C5b-9 that is generated is measured by contacting the mixture with an enzyme conjugated anti-C5b-9 antibody and a fluorogenic substrate and measuring the fluorescence at the appropriate wavelength. As a control, the test serum is incubated in the absence of the test compound. In some embodiments, the test serum is a C5-deficient serum reconstituted with a C5 polypeptide. In some embodiments, the C5 polypeptide is a variant C5 polypeptide. In some embodiments, the C5 polypeptide is a wild-type C5 polypeptide (SEQ ID NO:3). In some embodiments, the test serum is pooled normal human serum (PNHS). In some embodiments, the test serum is pooled non-responder human serum.

In some embodiments, C5 activity, or inhibition thereof, is quantified using a CH50eq assay. The CH50eq assay is a method for measuring the total classical complement activity in serum. This test is a lytic assay, which uses antibody-sensitized erythrocytes as the activator of the classical complement pathway and various dilutions of the test serum to determine the amount required to give 50% lysis (CH50). The percent hemolysis can be determined, for example, using a spectrophotometer. The CH50eq assay provides an indirect measure of terminal complement complex (TCC) formation, since the TCC themselves are directly responsible for the hemolysis that is measured.

The assay is well known and commonly practiced by those of skill in the art. Briefly, to activate the classical complement pathway, undiluted serum samples (e.g., reconstituted human serum samples) are added to microassay wells containing the antibody-sensitized erythrocytes to thereby generate TCC. Next, the activated sera are diluted in microassay wells, which are coated with a capture reagent (e.g., an antibody that binds to one or more components of the TCC). The TCC present in the activated samples bind to the monoclonal antibodies coating the surface of the microassay wells. The wells are washed and to each well is added a detection reagent that is detectably labeled and recognizes the bound TCC. The detectable label can be, e.g., a fluorescent label or an enzymatic label. The assay results are expressed in CH50 unit equivalents per milliliter (CH50 U Eq/mL).

C3 and C4 are both key components of classical C5 convertase, and C3 is also a key component of alternative C5 convertase. These convertases are required for the conversion of C5 into C5a and C5b. The ability to block C5 binding to C3 and/or C4 is thus a desirable property for a C5 antagonist. Therefore, in some embodiments, characterization of C5 antagonist comprises measuring the ability of the test compound to block C5 binding to C3 and/or C4 by using any of the immunological and biochemical methods to determine binding known in the art or as described herein.

A variety of assay formats can be used and will be understood as such by a skilled artisan.

### Test Compounds

A test compound described herein can be, e.g., a small molecule, a protein, a protein fragment, a polypeptide, a peptide, a polypeptide analog, a peptidomimetic, a nucleic acid, a nucleic acid analog, an aptamer including but not limited to an RNA aptamer including an L-RNA aptamer, a spiegelmer, a locked nucleic acid (LNA), a peptide nucleic acid (PNA), or an antibody. In some embodiments, the small molecule can be a non-antibody antigen-binding protein, e.g., one of the antibody-related scaffold protein constructs as described in Hey et al. (2005) TRENDS in Biotechnology 23(1):514-522.

“Small molecule” as used herein, is meant to refer to an agent, which preferably has a molecular weight of less than about 6 kDa and more preferably less than about 2.5 kDa. Many pharmaceutical companies have extensive libraries of chemical and/or biological mixtures comprising arrays of small molecules, often fungal, bacterial, or algal extracts, which can be screened with any of the assays of the application. This application contemplates using, among other things, small chemical libraries, peptide libraries, or collections of natural products. Tan et al. described a library with over two million synthetic compounds that is compatible with miniaturized cell-based assays (J. Am. Chem. Soc. (1998) 120:8565-8566). It is within the scope of this application that such a library may be used to screen for inhibitors of human complement component C5. There are numerous commercially available compound libraries, such as the Chembridge DIVERSet. Libraries are also available from academic investigators, such as the Diversity set from the NCI developmental therapeutics program. Rational drug design may also be employed. For example, rational drug design can employ the use of crystal or solution structural information on the human complement component C5 protein. See, e.g., the structures described in Hagemann et al. (2008) J Biol Chem 283(12):7763-75; Zuiderweg et al. (1989) Biochemistry 28(1):172-85 and Laursen et al. (2011) EMBO J 30(3):606-616. Rational drug design can also be achieved based on known compounds, e.g., a known inhibitor of C5 (e.g., an antibody, or antigen-binding fragment thereof, that binds to a human complement component C5 protein). Rational drug design can be further based on the identity and location of mutated residues in variant C5 polypeptides that confer resistance to C5 antagonists. For example, in some embodiments the test compound can be rationally designed to bind an epitope of C5 set forth between or comprising residues 872 and 892 of SEQ ID NO:2 or 47, e.g., an epitope comprising at least five (e.g., at least six, seven, eight, nine, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 125, 150, 175, 200, 250, 300, 350, 400, 450, 500, 550, 600, 650, 700, 750, 800, or 850) consecutive amino acids of SEQ ID NO:2 or 47, inclusive of amino acid 885. Test compounds contemplated by the present invention include small molecules, polypeptides, polypeptide analogs, peptidomimetics, nucleic acids, nucleic acid analogs, aptamers, and antibodies.

In some embodiments, the test compound is a peptidomimetic. Peptidomimetics can be compounds in which at least a portion of a subject polypeptide is modified, and the three dimensional structure of the peptidomimetic remains substantially the same as that of the subject polypeptide. Peptidomimetics may be analogues of a subject polypeptide of the disclosure that are, themselves, polypeptides containing one or more substitutions or other modifications within the subject polypeptide sequence. Alternatively, at least a portion of the subject polypeptide sequence may be replaced with a nonpeptide structure, such that the three-dimensional structure of the subject polypeptide is substantially retained. In other words, one, two or three amino acid residues within the subject polypeptide sequence may be replaced by a non-peptide structure. In addition, other peptide portions of the subject polypeptide may, but need not, be replaced with a non-peptide structure. Peptidomimetics (both peptide and non-peptidyl analogues) may have improved properties (e.g., decreased proteolysis, increased retention or increased bioavailability). Peptidomimetics generally have improved oral availability, which makes them especially suited to treatment of disorders in a human or animal. It should be noted that peptidomimetics may or may not have similar two-dimensional chemical structures, but share common three-dimensional structural features and geometry. Each peptidomimetic may further have one or more unique additional binding elements.

In some embodiments, the test compound is an aptamer. Aptamers are short oligonucleotide sequences that can be used to recognize and specifically bind almost any molecule, including cell surface proteins. The systematic evolution of ligands by exponential enrichment (SELEX) process is powerful and can be used to readily identify such aptamers. Aptamers can be made for a wide range of proteins of importance for therapy and diagnostics, such as growth factors and cell surface antigens. These oligonucleotides bind their targets with similar affinities and specificities as antibodies do (see, e.g., Ulrich (2006) Handb Exp Pharmacol. 173:305-326).

In some embodiments, the test compound is an antibody, or antigen-binding fragment thereof. Suitable methods for producing an antibody, or antigen-binding fragments thereof, in accordance with the disclosure are known in the art (see, e.g., U.S. Pat. No. 6,355,245 and WO 2010/015608) and described herein. For example, monoclonal anti-C5 antibodies may be generated using complement component C5-expressing cells, a C5 polypeptide, or an antigenic fragment of C5 polypeptide, as an immunogen, thus raising an immune response in animals from which antibody-producing cells and in turn monoclonal antibodies may be isolated. The sequence of such antibodies may be determined and the antibodies or variants thereof produced by recombinant techniques. Recombinant techniques may be used to produce chimeric, CDR-grafted, humanized and fully human antibodies based on the sequence of the monoclonal antibodies as well as polypeptides capable of binding to human complement component C5.

Moreover, antibodies derived from recombinant libraries (“phage antibodies”) may be selected using C5-expressing cells, or polypeptides derived therefrom, as bait to isolate the antibodies or polypeptides on the basis of target specificity. The production and isolation of non-human and chimeric anti-C5 antibodies are well within the purview of the skilled artisan.

### Diagnostic Assays

The present disclosure provides diagnostic and prognostic methods for predicting the responsiveness of a subject to treatment with a C5 antagonist based on detecting the presence or absence of at least one mutation in the C5 gene. For example, detecting the presence of a mutation in the C5 gene in or around the region encoding the binding pocket of C5 antagonists such as eculizumab (located within the sequence set forth in SEQ ID NO:4) is useful for determining if a subject is likely to respond to treatment with the C5 antagonist. Based on the prognostic information, a clinician can recommend a treatment regimen.

In one embodiment, the method comprises determining the presence of mutations in the C5 gene (Table 1). The mutation can comprise a deletion, an insertion, or a substitution. A subject identified as having a mutation in the C5 gene is likely to be non-responsive to treatment with certain C5 antagonists. In addition, knowledge of the identity of a particular allele (i.e. the genetic profile) in a subject allows customization of therapy in subjects suffering from a C5-associated disorder, enabling matching the subject's genetic profile to a particular treatment regimen. For example, if a subject is identified using the methods of the present disclosure as a likely non-responder to eculizumab treatment, an alternate C5 antagonist which binds C5 at a site different from eculizumab can be prescribed.

The method of this invention relates to nucleic acid molecules containing mutations, methods and reagents for the detection of the changes in the wild-type sequence of C5, uses of these mutations for the development of detection reagents, and assays or kits that utilize such reagents. Detection of nucleic acids encoding C5, as well as nucleic acids involved in the expression or stability of polypeptides or transcripts are also encompassed by the invention. General methods of nucleic acid detection are provided below.

Sample nucleic acid for use in the diagnostic and prognostic methods can be obtained from any cell type or tissue of a subject, if the sample nucleic acid is genomic DNA. If the sample nucleic acid is mRNA, the sample must be obtained from the cell type or tissues of a subject in which the mRNA is expressed. Similarly, if C5 protein or peptide is to be detected, the sample must be obtained from the cell type or tissue in which C5 expression is located, either expressed within the cell type or tissue, or translocated to the cell type or tissue.

For example, a subject's bodily fluid (e.g. blood) can be obtained by known techniques (e.g., venipuncture). Alternatively, nucleic acid tests can be performed on dry samples (e.g., hair or skin). Fetal nucleic acid samples can be obtained from maternal blood as described in International Patent Application No. WO 91/07660 to Bianchi.

Diagnostic procedures can also be performed in situ directly upon tissue sections (fixed and/or frozen) of patient tissue obtained from biopsies or resections, such that no nucleic acid purification is necessary. Nucleic acid reagents can be used as probes and/or primers for such in situ procedures (see, for example, Nuovo (1992) “PCR In Situ Hybridization: Protocols And Applications”, Raven Press, NY).

In one embodiment, the mutations of the present invention occur in the gene encoding the C5 polypeptide identified in Table 1 or fragments thereof or complements thereof. The probes of the present invention will preferably be “biologically active” with respect to either a structural attribute, such as the capacity of a nucleic acid to hybridize to another nucleic acid molecule or to be used by a polymerase as a primer. Alternatively, such an attribute can be catalytic, and thus involve the capacity of the agent to mediate a chemical reaction or response.

In one embodiment, the identity of at least one mutation site in C5 is determined. As used herein a mutation site includes one or more nucleotide substitutions, deletions, insertions, or base changes at a particular site in a nucleic acid sequence. In some embodiments, the identity of between about one and about six mutation sites is determined, though the identification of other numbers of sites is also possible. In some embodiments, the mutations and molecules of the present invention are utilized in determining the identity of at least one mutation site of the C5 and using that identity as a predictor of non-responsiveness to treatment with a C5 antagonist. The type of mutation present can also dictate the appropriate drug selection.

Methods of detecting mutations in the C5 gene as described herein are known in the art. Such methods include, but are not limited to, DNA sequencing, restriction fragment length polymorphism (RFLP) analysis, allele specific oligonucleotide (ASO) analysis, Denaturing/Temperature Gradient Gel Electrophoresis (DGGE/TGGE), Single-Strand Conformation Polymorphism (SSCP) analysis, Dideoxy fingerprinting (ddF), pyrosequencing analysis, acycloprime analysis, Reverse dot blot, GeneChip microarrays, Dynamic allele-specific hybridization (DASH), Peptide nucleic acid (PNA) and locked nucleic acids (LNA) probes, TaqMan, Molecular Beacons, Intercalating dye, FRET primers, AlphaScreen, SNPstream, genetic bit analysis (GBA), Multiplex minisequencing, SNaPshot, MassEXTEND, MassArray, GOOD assay, Microarray miniseq, arrayed primer extension (APEX), Microarray primer extension, Tag arrays, Coded microspheres, Template-directed incorporation (TDI), fluorescence polarization, Colorimetric oligonucleotide ligation assay (OLA), Sequence-coded OLA, Microarray ligation, Ligase chain reaction, Padlock probes, Rolling circle amplification, and Invader assay.

### Kits

The disclosure also features articles of manufacture or kits, which include a container with a label; and a composition containing one or more specific C5 polypeptides or fragments thereof, and reagents, for use in identifying C5 antagonists.

The disclosure also features articles of manufacture or kits, which include a container with a label; and a composition containing primer sequences, and reagents, for use in determining the presence or absence of a particular mutation in a sample obtained from a subject.

### EXAMPLES

While in no way intended to be limiting, exemplary methods of screening for novel compounds that inhibit complement activity are elaborated on below and exemplified in the working Examples.

### Example 1

Some patients have been identified as being non-responsive to C5 antagonist therapy, such as eculizumab treatment. The ability of C5 from such non-responder to bind known C5 antagonists such as eculizumab, relative to the ability of C5 from a subject responsive to known C5 antagonist therapy can be determined by using a binding assay. A lower binding of the non-responder C5 to the C5 antagonist as compared to the binding of the responder (wild-type) C5 to the antagonist indicates the presence of a mutation that directly or indirectly alters the epitope on C5 recognized by the C5 antagonist. Compound libraries may be screened for compounds that have a greater binding affinity for the C5 from the responder compared to the C5 from the non-responder. Such a compound is likely to bind C5 in or around the region that is involved in recognition by the C5 antagonist and hence is likely involved in recognition and binding by the C5 convertases. Such a compound may be formulated to provide suitable pharmaceutical compositions for treating patients who have wild-type C5.

### Example 2

Some patients have been identified as being non-responsive to known C5 antagonists such as eculizumab. The ability of C5 from the non-responder to bind known C5 antagonists, relative to the ability of C5 from a responder to bind the C5 antagonist, is determined by using a binding assay. A lower binding of the non-responder C5 to the known C5 antagonist as compared to the binding of the wild-type C5 to the known antagonist indicates the presence of a mutation that directly or indirectly alters the epitope on C5 recognized by the known C5 antagonist. Compound libraries are screened for compounds that have the ability to bind the C5 from the non-responder with a higher affinity than they bind to wild-type C5. Such compounds are likely to bind to the area of the mutation and thus to the region bound by the known C5 antagonist and will likely be useful in inhibiting binding of C5 to a C5 convertase. Such compounds may be further tested for their ability to inhibit C5 activity. Such a compound may be formulated to provide suitable pharmaceutical compositions useful for treating the non-responder.

### Example 3

The C5 gene is isolated from a sample obtained from a patient who is non-responsive to treatment with eculizumab. Nucleic acid for the variant C5 gene from the non-responder and nucleic acid encoding wild-type C5 (Table 1 and SEQ ID NO:1) are each cloned into separate expression vectors and transfected into mammalian host cells for recombinant production of the two C5 polypeptides. The variant and wild-type C5 polypeptides are used in a screening assay to identify compounds that have a greater affinity for the variant C5 polypeptide compared to the wild-type polypeptide. Such a compound is likely to bind the variant C5 polypeptide in, or adjacent to, the epitope recognized by eculizumab and is further tested for an ability to inhibit complement-mediated hemolysis. The compound is used in a pharmaceutical composition to treat patients who are non-responsive to eculizumab.

### Example 4

The C5 gene is isolated from a sample obtained from a patient who is non-responsive to treatment with eculizumab. Nucleic acid for the variant C5 gene from the non-responder and nucleic acid encoding wild-type C5 (Table 1 and SEQ ID NO:1) are each cloned into separate expression vectors and transfected into mammalian host cells for recombinant production of the two C5 polypeptides. The variant and wild-type C5 polypeptides are used in a screening assay to identify compounds that have a greater affinity for the wild-type C5 polypeptide compared to the variant polypeptide. Such a compound is likely to bind the wild-type C5 polypeptide in, or adjacent to, the epitope recognized by eculizumab and is further tested for an ability to inhibit complement-mediated hemolysis. The compound is used in a pharmaceutical composition to treat patients who are wild-type for C5.

TABLE 1

| Exon | NCBI Reference Sequence | SEQ ID NO: |
|---|---|---|
| 1 | NG_007364.1 (5001 to 5095) | 5 |
| 2 | NG_007364.1 (8894 to 9086) | 6 |
| 3 | NG_007364.1 (12111 to 12273 ) | 7 |
| 4 | NG_007364.1 (17326 to 17396) | 8 |
| 5 | NG_007364.1 (20383 to 20474) | 9 |
| 6 | NG_007364.1(23082 to 23164) | 10 |
| 7 | NG_007364.1 (19790 to 19880) | 11 |
| 8 | NG_007364.1 (28003 to 28117) | 12 |
| 9 | NG_007364.1 (29697 to 29823) | 13 |
| 10 | NG_007364.1 (31758 to 31873) | 14 |
| 11 | NG_007364.1 (33583 to 33768) | 15 |
| 12 | NG_007364.1 (35098 to 35301) | 16 |
| 13 | NG_007364.1 (37425 to 37634) | 17 |
| 14 | NG_007364.1 (32766 to 32915) | 18 |
| 15 | NG_007364.1 (38894 to 39023) | 19 |
| 16 | NG_007364.1 (40016 to 40078) | 20 |
| 17 | NG_007364.1 (41207 to 41404) | 21 |
| 18 | NG_007364.1 (46779 to 46869) | 22 |
| 19 | NG_007364.1 (48300 to 48373) | 23 |
| 20 | NG_007364.1 (49219 to 49358) | 24 |
| 21 | NG_007364.1 (57503 to 57730) | 25 |
| 22 | NG_007364.1 (58977 to 59037) | 26 |
| 23 | NG_007364.1 (63997 to 64086) | 27 |
| 24 | NG_007364.1 (65497 to 65709) | 28 |
| 25 | NG_007364.1 (66156 to 66231) | 29 |
| 26 | NG_007364.1 (72463 to 72622) | 30 |
| 27 | NG_007364.1 (73338 to 73433) | 31 |
| 28 | NG_007364.1 (75023 to 75194) | 32 |
| 29 | NG_007364.1 (78372 to 78577) | 33 |
| 30 | NG_007364.1 (80346 to 80498) | 34 |
| 31 | NG_007364.1 (83285 to 83347) | 35 |
| 32 | NG_007364.1 (85026 to 85107) | 36 |
| 33 | NG_007364.1 (86159 to 86216) | 37 |
| 34 | NG_007364.1 (91539 to 91641) | 38 |
| 35 | NG_007364.1 (92310 to 92384) | 39 |
| 36 | NG_007364.1 (92501 to 92606) | 40 |
| 37 | NG_007364.1 (93712 to 93795) | 41 |
| 38 | NG_007364.1 (94940 to 95029) | 42 |
| 39 | NG_007364.1 (97909 to 97992) | 43 |
| 40 | NG_007364.1 (101409 to 101547) | 44 |
| 41 | NG_007364.1 (102409 to 102942) | 45 |

### Example 5

The variant and wild-type C5 polypeptides of Example 3 are used to screen for compounds that potentiate or inhibit conversion of C5 into C5a and C5b. A mixture of a C5 polypeptide and a test compound is added to C5-depleted human serum and the amount of C5b generated is measured by a CH50 Eq assay as follows. The undiluted serum samples are added to microassay wells containing antibody-sensitized erythrocytes to thereby generate TCC. Next, the activated sera are diluted in microassay wells, which are coated with a capture reagent (e.g., an antibody that binds to one or more components of the TCC). The TCC present in the activated samples binds to the monoclonal antibodies coating the surface of the microassay wells. The wells are washed and to each well is added a detection reagent that is detectably labeled and recognizes the bound TCC. The detectable label can be, e.g., a fluorescent label or an enzymatic label. The assay results are expressed in CH50 unit equivalents per milliliter (CH50 U Eq/mL). The amount of C5b generated, as determined by the generation of TCC, is compared against the amount of C5b generated in the absence of a test compound. It will be understood by a person of ordinary skill in the art that it is not necessary for the control to be performed alongside the test assay and a historical control would work as well.

### Example 6

The variant and wild-type C5 polypeptides of Example 3 are used to screen for compounds that bind both the wild-type and variant C5 polypeptides and further inhibit the cleavage of C5 into C5a and C5b. Such a compound can be used in a pharmaceutical composition to treat patients who are non-responsive to eculizumab as well as those with wild-type C5 who are responsive to eculizumab.

While the present disclosure has been described with reference to the specific embodiments thereof, it should be understood by those skilled in the art that various changes may be made and equivalents may be substituted without departing from the true spirit and scope of the disclosure. In addition, many modifications may be made to adapt a particular situation, material, composition of matter, process, process step or steps, to the objective, spirit and scope of the present disclosure. All such modifications are intended to be within the scope of the disclosure.

Sequence Listing:

SEQ ID NO: 1 (human C5 cDNA sequence; NCBI Accession No. M57729 and
Haviland et al.)

| | |
|---|---|
| 1 | ctacctccaa ccatgggcct tttgggaata ctttgttttt taatcttcct ggggaaaacc |
| 61 | tggggacagg agcaaacata tgtcatttca gcaccaaaaa tattccgtgt tggagcatct |
| 121 | gaaaatattg tgattcaagt ttatggatac actgaagcat ttgatgcaac aatctctatt |
| 181 | aaaagttatc ctgataaaaa atttagttac tcctcaggcc atgttcattt atcctcagag |
| 241 | aataaattcc aaaactctgc aatcttaaca atacaaccaa aacaattgcc tggaggacaa |
| 301 | aacccagttt cttatgtgta tttggaagtt gtatcaaagc atttttcaaa atcaaaaaga |
| 361 | atgccaataa cctatgacaa tggatttctc ttcattcata cagacaaacc tgtttatact |
| 421 | ccagaccagt cagtaaaagt tagagtttat tcgttgaatg acgacttgaa gccagccaaa |
| 481 | agagaaactg tcttaacctt catagatcct gaaggatcag aagttgacat ggtagaagaa |
| 541 | attgatcata ttggaattat ctcttttcct gacttcaaga ttccgtctaa tcctagatat |
| 601 | ggtatgtgga cgatcaaggc taaatataaa gaggactttt caacaactgg aaccgcatat |
| 661 | tttgaagtta aagaatatgt cttgccacat ttttctgtct caatcgagcc agaatataat |
| 721 | ttcattggtt acaagaactt taagaatttt gaaattacta taaaagcaag atatttttat |
| 781 | aataaagtag tcactgaggc tgacgtttat atcacatttg gaataagaga agacttaaaa |
| 841 | gatgatcaaa aagaaatgat gcaaacagca atgcaaaaca caatgttgat aaatggaatt |
| 901 | gctcaagtca catttgattc tgaaacagca gtcaaagaac tgtcatacta cagtttagaa |
| 961 | gatttaaaca acaagtacct ttatattgct gtaacagtca tagagtctac aggtggattt |
| 1021 | tctgaagagg cagaaatacc tggcatcaaa tatgtcctct ctccctacaa actgaatttg |
| 1081 | gttgctactc ctcttttcct gaagcctggg attccatatc ccatcaaggt gcaggttaaa |
| 1141 | gattcgcttg accagttggt aggaggagtc ccagtaatac tgaatgcaca aacaattgat |
| 1201 | gtaaaccaag agacatctga cttggatcca agcaaaagtg taacacgtgt tgatgatgga |
| 1261 | gtagcttcct ttgtgcttaa tctcccatct ggagtgacgg tgctggagtt taatgtcaaa |
| 1321 | actgatgctc cagatcttcc agaagaaaat caggccaggg aaggttaccg agcaatagca |
| 1381 | tactcatctc tcagccaaag ttacctttat attgattgga ctgataacca taaggctttg |
| 1441 | ctagtgggag aacatctgaa tattattgtt acccccaaaa gcccatatat tgacaaaata |
| 1501 | actcactata attacttgat tttatccaag ggcaaaatta tccattttgg cacgagggag |
| 1561 | aaattttcag atgcatctta tcaaagtata aacattccag taacacagaa catggttcct |
| 1621 | tcatcccgac ttctggtcta ttatatcgtc acaggagaac agacagcaga attagtgtct |
| 1681 | gattcagtct ggttaaatat tgaagaaaaa tgtggcaacc agctccaggt tcatctgtct |
| 1741 | cctgatgcag atgcatattc tccaggccaa actgtgtctc ttaatatggc aactggaatg |
| 1801 | gattcctggg tggcattagc agcagtggac agtgctgtgt atggagtcca aagaggagcc |
| 1861 | aaaaagccct tggaaagagt atttcaattc ttagagaaga gtgatctggg ctgtggggca |
| 1921 | ggtggtggcc tcaacaatgc caatgtgttc cacctagctg gacttacctt cctcactaat |
| 1981 | gcaaatgcag atgactccca agaaaatgat gaaccttgta aagaaattct caggccaaga |
| 2041 | agaacgctgc aaaagaagat agaagaaata gctgctaaat ataaacattc agtagtgaag |
| 2101 | aaatgttgtt acgatggagc ctgcgttaat aatgatgaaa cctgtgagca gcgagctgca |
| 2161 | cggattagtt tagggccaag atgcatcaaa gctttcactg aatgttgtgt cgtcgcaagc |
| 2221 | cagctccgtg ctaatatctc tcataaagac atgcaattgg gaaggctaca catgaagacc |
| 2281 | ctgttaccag taagcaagcc agaaattcgg agttattttc cagaaagctg gttgtgggaa |
| 2341 | gttcatcttg ttcccagaag aaaacagttg cagtttgccc tacctgattc tctaaccacc |
| 2401 | tgggaaattc aaggcattgg catttcaaac actggtatat gtgttgctga tactgtcaag |
| 2461 | gcaaaggtgt tcaaagatgt cttcctggaa atgaatatac catattctgt tgtacgagga |
| 2521 | gaacagatcc aattgaaagg aactgtttac aactatagga cttctgggat gcagttctgt |
| 2581 | gttaaaatgt ctgctgtgga gggaatctgc acttcggaaa gcccagtcat tgatcatcag |
| 2641 | ggcacaaagt cctccaaatg tgtgcgccag aaagtagagg gctcctccag tcacttggtg |
| 2701 | acattcactg tgcttcctct ggaaattggc cttcacaaca tcaatttttc actggagact |
| 2761 | tggtttggaa aagaaatctt agtaaaaaca ttacgagtgg tgccagaagg tgtcaaaagg |
| 2821 | gaaagctatt ctggtgttac tttggatcct aggggtattt atggtaccat tagcagacga |
| 2881 | aaggagttcc catacaggat acccttagat ttggtcccca aaacagaaat caaaaggatt |
| 2941 | ttgagtgtaa aaggactgct tgtaggtgag atcttgtctg cagttctaag tcaggaaggc |
| 3001 | atcaatatcc taacccacct ccccaaaggg agtgcagagg cggagctgat gagcgttgtc |
| 3061 | ccagtattct atgtttttca ctacctggaa acaggaaatc attggaacat ttttcattct |
| 3121 | gacccattaa ttgaaaagca gaaactgaag aaaaaattaa aagaagggat gttgagcatt |
| 3181 | atgtcctaca gaaatgctga ctactcttac agtgtgtgga agggtggaag tgctagcact |
| 3241 | tggttaacag cttttgcttt aagagtactt ggacaagtaa ataaatacgt agagcagaac |
| 3301 | caaaattcaa tttgtaattc tttattgtgg ctagttgaga attatcaatt agataatgga |
| 3361 | tctttcaagg aaaattcaca gtatcaacca ataaaattac agggtacctt gcctgttgaa |
| 3421 | gcccgagaga acagcttata tcttacagcc tttactgtga ttggaattag aaaggctttc |
| 3481 | gatatatgcc ccctggtgaa aatcgacaca gctctaatta aagctgacaa ctttctgctt |
| 3541 | gaaaatacac tgccagccca gagcaccttt acattggcca tttctgcgta tgctctttcc |
| 3601 | ctgggagata aaactcaccc acagtttcgt tcaattgttt cagctttgaa gagagaagct |
| 3661 | ttggttaaag gtaatccacc catttatcgt ttttggaaag acaatcttca gcataaagac |
| 3721 | agctctgtac ctaacactgg tacggcacgt atggtagaaa caactgccta tgctttactc |
| 3781 | accagtctga acttgaaaga tataaattat gttaacccag tcatcaaatg gctatcagaa |
| 3841 | gagcagaggt atggaggtgg cttttattca acccaggaca ccatcaatgc cattgagggc |
| 3901 | ctgacggaat attcactcct ggttaaacaa ctccgcttga gtatggacat cgatgtttct |
| 3961 | tacaagcata aaggtgcctt acataattat aaaatgacag acaagaattt ccttgggagg |
| 4021 | ccagtagagg tgcttctcaa tgatgacctc attgtcagta caggatttgg cagtggcttg |
| 4081 | gctacagtac atgtaacaac tgtagttcac aaaaccagta cctctgagga agtttgcagc |
| 4141 | ttttatttga aaatcgatac tcaggatatt gaagcatccc actacagagg ctacggaaac |
| 4201 | tctgattaca aacgcatagt agcatgtgcc agctacaagc ccagcaggga agaatcatca |
| 4261 | tctggatcct ctcatgcggt gatggacatc tccttgccta ctggaatcag tgcaaatgaa |
| 4321 | gaagacttaa aagcccttgt ggaaggggtg gatcaactat tcactgatta ccaaatcaaa |
| 4381 | gatggacatg ttattctgca actgaattcg attccctcca gtgatttcct ttgtgtacga |
| 4441 | ttccggatat ttgaactctt tgaagttggg tttctcagtc ctgccacttt cacagtttac |
| 4501 | gaataccaca gaccagataa acagtgtacc atgttttata gcacttccaa tatcaaaatt |
| 4561 | cagaaagtct gtgaaggagc cgcgtgcaag tgtgtagaag ctgattgtgg gcaaatgcag |
| 4621 | gaagaattgg atctgacaat ctctgcagag acaagaaaac aaacagcatg taaaccagag |
| 4681 | attgcatatg cttataaagt tagcatcaca tccatcactg tagaaaatgt ttttgtcaag |
| 4741 | tacaaggcaa cccttctgga tatctacaaa actggggaag ctgttgctga gaaagactct |
| 4801 | gagattacct tcattaaaaa ggtaacctgt actaacgctg agctggtaaa aggaagacag |
| 4861 | tacttaatta tgggtaaaga agccctccag ataaaataca atttcagttt caggtacatc |
| 4921 | taccctttag attccttgac ctggattgaa tactggccta gagacacaac atgttcatcg |
| 4981 | tgtcaagcat ttttagctaa tttagatgaa tttgccgaag atatcttttt aaatggatgc |
| 5041 | taaaattcct gaagttcagc tgcatacagt ttgcacttat ggactcctgt tgttgaagtt |
| 5101 | cgtttttttg ttttcttctt tttttaaaca ttcatagctg gtcttatttg taaagctcac |
| 5161 | tttacttaga attagtggca cttgctttta ttagagaatg atttcaaatg ctgtaacttt |
| 5221 | ctgaaataac atggccttgg agggcatgaa gacagatact cctccaaggt tattggacac |
| 5281 | cggaaacaat aaattggaac acctcctcaa acctaccact caggaatgtt tgctggggcc |
| 5341 | gaaagaacag tccattgaaa gggagtatta caaaaacatg gcctttgctt gaaagaaaat |
| 5401 | accaaggaac aggaaactga tcattaaagc ctgagtttgc tttc |

SEQ ID NO: 2 (human C5 protein sequence; NCBI Accession No. AAA51925 and Haviland
et al.)

| | |
|---|---|
| 1 | mgllgilcfl iflgktwgqe qtyvisapki frvgaseniv iqvygyteaf datisiksyp |
| 61 | dkkfsyssgh vhlssenkfq nsailtiqpk qlpggqnpvs yvylevvskh fskskrmpit |
| 121 | ydngflfiht dkpvytpdqs vkvrvyslnd dlkpakretv ltfidpegse vdmveeidhi |
| 181 | giisfpdfki psnprygmwt ikakykedfs ttgtayfevk eyvlphfsys iepeynfigy |
| 241 | knfknfeiti karyfynkvv teadvyitfg iredlkddqk emmqtamqnt mlingiaqvt |
| 301 | fdsetavkel syysledlnn kylyiavtvi estggfseea eipgikyvls pyklnlvatp |
| 361 | lflkpgipyp ikvqvkdsld qlvggvpvil naqtidvnqe tsdldpsksv trvddgvasf |
| 421 | vlnlpsgvtv lefnvktdap dlpeenqare gyraiayssl sqsylyidwt dnhkallvge |
| 481 | hlniivtpks pyidkithyn ylilskgkii hfgtrekfsd asyqsinipv tqnmvpssrl |
| 541 | lvyyivtgeq taelvsdsvw lnieekcgnq lqvhlspdad ayspgqtvsl nmatgmdswv |
| 601 | alaavdsavy gvqrgakkpl ervfqfleks dlgcgagggl nnanvfhlag ltfltnanad |
| 661 | dsgendepck eilrprrtlq kkieeiaaky khsvvkkccy dgacvnndet ceqraarisl |
| 721 | gprcikafte ccvvasqlra nishkdmqlg rlhmktllpv skpeirsyfp eswlwevhlv |
| 781 | prrkqlqfal pdslttweiq gigisntgic vadtvkakvf kdvflemnip ysvvrgeqiq |
| 841 | lkgtvynyrt sgmqfcvkms avegictses pvidhqgtks skcvrqkveg ssshlvtftv |
| 901 | 1pleiglhni nfsletwfgk eilvktlrvv pegvkresys gvtldprgiy gtisrrkefp |
| 961 | yripldlvpk teikrilsvk gllvgeilsa vlsqeginil thlpkgsaea elmsvvpvfy |
| 1021 | vfhyletgnh wnifhsdpli ekqklkkklk egmlsimsyr nadysysvwk ggsastwlta |
| 1081 | falrvlgqvn kyveqnqnsi cnsllwlven yqldngsfke nsqyqpiklq gtlpvearen |
| 1141 | slyltaftvi girkafdicp lvkidtalik adnfllentl paqstftlai sayalslgdk |
| 1201 | thpqfrsivs alkrealvkg nppiyrfwkd nlqhkdssvp ntgtarmvet tayalltsln |
| 1261 | lkdinyvnpv ikwlseeqry gggfystqdt inaiegltey sllvkqlrls mdidvsykhk |
| 1321 | galhnykmtd knflgrpvev llnddlivst gfgsglatvh vttvvhktst seevcsfylk |
| 1381 | idtqdieash yrgygnsdyk rivacasykp sreesssgss havmdislpt gisaneedlk |
| 1441 | alvegvdqlf tdyqikdghv ilqlnsipss dflcvrfrif elfevgflsp atftvyeyhr |
| 1501 | pdkqctmfys tsnikiqkvc egaackcvea dcgqmqeeld ltisaetrkq tackpeiaya |
| 1561 | ykvsitsitv envfvkykat lldiyktgea vaekdseitf ikkvtctnae lvkgrqylim |
| 1621 | gkealqikyn fsfryiypld sltwieywpr dttcsscqaf lanldefaed iflngc |

SEQ ID NO: 3 (human C5 protein lacking the amino terminal leader sequence)

| | |
|---|---|
| 1 | qeqtyvisap kifrvgasen iviqvygyte afdatisiks ypdkkfsyss |
| | ghvhlssenk |
| 61 | fqnsailtiq pkqlpggqnp vsyvylevvs khfskskrmp itydngflfi |
| | htdkpvytpd |
| 121 | qsvkvrvysl nddlkpakre tvltfidpeg sevdmveeid higiisfpdf |
| | kipsnprygm |
| 181 | wtikakyked fsttgtayfe vkeyvlphfs vsiepeynfi gyknfknfei |
| | tikaryfynk |
| 241 | Vvteadvyit fgiredlkdd qkemmqtamq ntmlingiaq Vtfdsetavk |
| | elsyysledl |
| 301 | nnkylyiavt viestggfse eaeipgikyv lspyklnlva Tplflkpgip |
| | ypikvqvkds |
| 361 | ldqlvggvpv ilnaqtidvn qetsdldpsk svtrvddgva sfvlnlpsgv |
| | tvlefnvktd |
| 421 | apdlpeenqa regyraiays slsqsylyid wtdnhkallv gehlniivtp |
| | kspyidkith |
| 481 | ynylilskgk iihfgtrekf sdasyqsini pvtqnmvpss rllvyyivtg |
| | eqtaelvsds |
| 541 | vwlnieekcg nqlqvhlspd adayspgqtv slnmatgmds wvalaavdsa |
| | vygvqrgakk |
| 601 | plervfqfle ksdlgcgagg glnnanvfhl agltfltnan addsqendep |
| | ckeilrprrt |
| 661 | lqkkieeiaa kykhsvvkkc cydgacvnnd etceqraari slgprcikaf |
| | teccvvasql |
| 721 | ranishkdmq lgrlhmktll pvskpeirsy fpeswlwevh lvprrkqlqf |
| | alpdslttwe |
| 781 | iqgigisntg icvadtvkak vfkdvflemn ipysvvrgeq iqlkgtvyny |
| | rtsgmqfcvk |
| 841 | msavegicts espvidhqgt ksskcvrqkv egssshlvtf tvlpleiglh |
| | ninfsletwf |
| 901 | gkeilvktlr vvpegvkres ysgvtldprg iygtisrrke fpyripldlv |
| | pkteikrils |
| 961 | vkgllvgeil savlsqegin ilthlpkgsa eaelmsvvpv fyvfhyletg |
| | nhwnifhsdp |
| 1021 | liekqklkkk lkegmlsims yrnadysysv wkggsastwl tafalrvlgq |
| | vnkyveqnqn |
| 1081 | sicnsllwlv enyqldngsf kensqyqpik lqgtlpvear enslyltaft |
| | vigirkafdi |
| 1141 | cplvkidtal ikadnfllen tlpaqstftl aisayalslg dkthpqfrsi |
| | vsalkrealv |
| 1201 | kgnppiyrfw kdnlqhkdss vpntgtarmv ettayallts lnlkdinyvn |
| | pvikwlseeq |
| 1261 | rygggfystq dtinaieglt eysllvkqlr lsmdidvsyk hkgalhnykm |
| | tdknflgrpv |
| 1321 | evllnddliv stgfgsglat vhvttvvhkt stseevcsfy lkidtqdiea |
| | shyrgygnsd |
| 1381 | ykrivacasy kpsreesssg sshavmdisl ptgisaneed lkalvegvdq |
| | lftdyqikdg |
| 1441 | hvilqlnsip ssdflcvrfr ifelfevgfl spatftvyey hrpdkqctmf |
| | ystsnikiqk |
| 1501 | vcegaackcv eadcgqmqee ldltisaetr kqtackpeia yaykvsitsi |
| | tvenvfvkyk |
| 1561 | atlldiyktg eavaekdsei tfikkvtctn aelvkgrqyl imgkealqik |
| | ynfsfryiyp |
| 1621 | ldsltwieyw prdttcsscq aflanldefa ediflngc |

SEQ ID NO: 4
VIDHQGTKSSKCVRQKVEGSS
SEQ ID NO: 46
KSSKC
SEQ ID NO: 47 (variant human C5 protein sequence comprising the R885H
substitution)

| | |
|---|---|
| 1 | mgllgilcfl iflgktwgqe qtyvisapki frvgaseniv iqvygyteaf datisiksyp |
| 61 | dkkfsyssgh vhlssenkfq nsailtiqpk qlpggqnpvs yvylevvskh fskskrmpit |
| 121 | ydngflfiht dkpvytpdqs vkvrvyslnd dlkpakretv ltfidpegse vdmveeidhi |
| 181 | giisfpdfki psnprygmwt ikakykedfs ttgtayfevk eyvlphfsys iepeynfigy |
| 241 | knfknfeiti karyfynkvv teadvyitfg iredlkddqk emmqtamqnt mlingiaqvt |
| 301 | fdsetavkel syysledlnn kylyiavtvi estggfseea eipgikyvls pyklnlvatp |
| 361 | lflkpgipyp ikvqvkdsld qlvggvpvil naqtidvnqe tsdldpsksv trvddgvasf |
| 421 | vlnlpsgvtv lefnvktdap dlpeenqare gyraiayssl sqsylyidwt dnhkallvge |
| 481 | hlniivtpks pyidkithyn ylilskgkii hfgtrekfsd asyqsinipv tqnmvpssrl |
| 541 | lvyyivtgeq taelvsdsvw lnieekcgnq lqvhlspdad ayspgqtvsl nmatgmdswv |
| 601 | alaavdsavy gvqrgakkpl ervfqfleks dlgcgagggl nnanvfhlag ltfltnanad |
| 661 | dsqendepck eilrprrtlq kkieeiaaky khsvvkkccy dgacvnndet ceqraarisl |
| 721 | gprcikafte ccvvasqlra nishkdmqlg rlhmktllpv skpeirsyfp eswlwevhlv |
| 781 | prrkqlqfal pdslttweiq gigisntgic vadtvkakvf kdvflemnip ysvvrgeqiq |
| 841 | lkgtvynyrt sgmqfcvkms avegictses pvidhqgtks skcvhqkveg ssshlvtftv |
| 901 | lpleiglhni nfsletwfgk eilvktlrvv pegvkresys gvtldprgiy gtisrrkefp |
| 961 | yripldlvpk teikrilsvk gllvgeilsa vlsqeginil thlpkgsaea elmsvvpvfy |
| 1021 | vfhyletgnh wnifhsdpli ekqklkkklk egmlsimsyr nadysysvwk ggsastwlta |
| 1081 | falrvlgqvn kyveqnqnsi cnsllwlven yqldngsfke nsqyqpiklq gtlpvearen |
| 1141 | slyltaftvi girkafdicp lvkidtalik adnfllentl paqstftlai sayalslgdk |
| 1201 | thpqfrsivs alkrealvkg nppiyrfwkd nlqhkdssvp ntgtarmvet tayalltsln |
| 1261 | lkdinyvnpv ikwlseeqry gggfystqdt inaiegltey sllvkqlrls mdidvsykhk |
| 1321 | galhnykmtd knflgrpvev llnddlivst gfgsglatvh vttvvhktst seevcsfylk |
| 1381 | idtqdieash yrgygnsdyk rivacasykp sreesssgss havmdislpt gisaneedlk |
| 1441 | alvegvdqlf tdyqikdghv ilqlnsipss dflcvrfrif elfevgflsp atftvyeyhr |
| 1501 | pdkqctmfys tsnikiqkvc egaackcvea dcgqmqeeld ltisaetrkq tackpeiaya |
| 1561 | ykvsitsitv envfvkykat lldiyktgea vaekdseitf ikkvtctnae lvkgrqylim |
| 1621 | gkealqikyn fsfryiypld sltwieywpr dttcsscqaf lanldefaed iflngc |

SEQ ID NO: 48 (variant human C5 protein comprising the R885H substitution and
lacking the amino terminal leader sequence)

| | |
|---|---|
| 1 | qeqtyvisap kifrvgasen iviqvygyte afdatisiks ypdkkfsyss |
| | ghvhlssenk |
| 61 | fqnsailtiq pkqlpggqnp vsyvylevvs khfskskrmp itydngflfi |
| | htdkpvytpd |
| 121 | qsvkvrvysl nddlkpakre tvltfidpeg sevdmveeid higiisfpdf |
| | kipsnprygm |
| 181 | wtikakyked fsttgtayfe vkeyvlphfs vsiepeynfi gyknfknfei |
| | tikaryfynk |
| 241 | Vvteadvyit fgiredlkdd qkemmqtamq ntmlingiaq Vtfdsetavk |
| | elsyysledl |
| 301 | nnkylyiavt viestggfse eaeipgikyv lspyklnlva Tplflkpgip |
| | ypikvqvkds |
| 361 | ldqlvggvpv ilnaqtidvn qetsdldpsk svtrvddgva sfvlnlpsgv |
| | tvlefnvktd |
| 421 | apdlpeenqa regyraiays slsqsylyid wtdnhkallv gehlniivtp |
| | kspyidkith |
| 481 | ynylilskgk iihfgtrekf sdasyqsini pvtqnmvpss rllvyyivtg |
| | eqtaelvsds |
| 541 | vwlnieekcg nqlqvhlspd adayspgqtv slnmatgmds wvalaavdsa |
| | vygvqrgakk |
| 601 | plervfqfle ksdlgcgagg glnnanvfhl agltfltnan addsqendep |
| | ckeilrprrt |
| 661 | lqkkieeiaa kykhsvvkkc cydgacvnnd etceqraari slgprcikaf |
| | teccvvasql |
| 721 | ranishkdmq lgrlhmktll pvskpeirsy fpeswlwevh lvprrkqlqf |
| | alpdslttwe |
| 781 | iqgigisntg icvadtvkak vfkdvflemn ipysvvrgeq iqlkgtvyny |
| | rtsgmqfcvk |
| 841 | msavegicts espvidhqgt ksskcvhqkv egssshlvtf tvlpleiglh |
| | ninfsletwf |
| 901 | gkeilvktlr vvpegvkres ysgvtldprg iygtisrrke fpyripldlv |
| | pkteikrils |
| 961 | vkgllvgeil savlsqegin ilthlpkgsa eaelmsvvpv fyvfhyletg |
| | nhwnifhsdp |
| 1021 | liekqklkkk lkegmlsims yrnadysysv wkggsastwl tafalrvlgq |
| | vnkyveqnqn |
| 1081 | sicnsllwlv enyqldngsf kensqyqpik lqgtlpvear enslyltaft |
| | vigirkafdi |
| 1141 | cplvkidtal ikadnfllen tlpaqstftl aisayalslg dkthpqfrsi |
| | vsalkrealv |
| 1201 | kgnppiyrfw kdnlqhkdss vpntgtarmv ettayallts lnlkdinyvn |
| | pvikwlseeq |
| 1261 | rygggfystq dtinaieglt eysllvkqlr lsmdidvsyk hkgalhnykm |
| | tdknflgrpv |
| 1321 | evllnddliv stgfgsglat vhvttvvhkt stseevcsfy lkidtqdiea |
| | shyrgygnsd |
| 1381 | ykrivacasy kpsreesssg sshavmdisl ptgisaneed lkalvegvdq |
| | lftdyqikdg |
| 1441 | hvilqlnsip ssdflcvrfr ifelfevgfl spatftvyey hrpdkqctmf |
| | ystsnikiqk |
| 1501 | vcegaackcv eadcgqmqee ldltisaetr kqtackpeia yaykvsitsi |
| | tvenvfvkyk |
| 1561 | atlldiyktg eavaekdsei tfikkvtctn aelvkgrqyl imgkealqik |
| | ynfsfryiyp |
| 1621 | ldsltwieyw prdttcsscq aflanldefa ediflngc |

What is claimed is:

1. A method of identifying a compound that binds to a wild-type C5 polypeptide at a region within or overlapping with the region of the wild-type C5 polypeptide bound by a known wild-type C5 antagonist, the method comprising:
(i) providing a variant C5 polypeptide to which the known wild-type C5 antagonist compound: (a) does not bind or (b) binds with lower affinity as compared to the affinity of the known wild-type C5 antagonist for the wild-type C5 polypeptide;
(ii) determining whether a test compound binds to the variant C5 polypeptide; and
(iii) determining whether the test compound binds to the wild-type C5 polypeptide;
wherein a test compound that binds to the wild-type C5 polypeptide, but not to the variant C5 polypeptide or a test compound that preferentially binds to the wild-type C5 polypeptide as compared to the variant C5 polypeptide, is indicative of a compound that binds to the wild-type C5 polypeptide at a region within or overlapping with the region of the wild-type C5 polypeptide bound by the known wild-type C5 antagonist.

2. The method of claim 1, further comprising selecting a test compound that binds to the wild-type C5 polypeptide, but not to the variant C5 polypeptide or a test compound that preferentially binds to the wild-type C5 polypeptide as compared to the variant C5 polypeptide.

3. The method of claim 1 or 2, wherein the test compound inhibits cleavage of C5 into fragments C5a and C5b.

4. The method of claim 1 or 2, further comprising determining whether the test compound inhibits the cleavage of C5 into fragments C5a and C5b.

5. The method of claim 4, wherein a hemolytic assay is used to determine whether the test compound inhibits the cleavage of C5 into fragments C5a and C5b.

6. The method of claim 1 or 2, wherein the wild-type C5 polypeptide comprises an amino acid sequence set forth in SEQ ID NO:2 or a fragment thereof.

7. The method of claim 1 or 2, wherein the variant C5 polypeptide comprises a deletion, an insertion, or a substitution, as compared to the wild-type C5 polypeptide.

8. The method of claim 7, wherein the deletion, insertion, or substitution is at a C5 convertase-binding site.

9. The method of claim 7, wherein the deletion, insertion, or substitution is present between residues 872 and 892 of SEQ ID NO:2.

10. The method of claim 7, wherein the deletion, insertion, or substitution is present at the epitope to which the known wild-type C5 antagonist binds.

11. The method of claim 1 or 2, wherein the variant C5 polypeptide is present in a subject non-responsive to treatment with the known C5 antagonist.

12. The method of claim 1 or 2, wherein the known wild-type C5 antagonist is an anti-C5 antibody or an antigen binding fragment thereof, a small molecule, a polypeptide, a polypeptide analog, a peptidomimetic, or an aptamer.

13. The method of claim 12, wherein the known wild-type C5 antagonist is eculizumab.

14. The method of claim 12, wherein the known wild-type C5 antagonist is pexelizumab.

15. The method of claim 12, wherein the known wild-type C5 antagonist is selected from the group consisting of MB12/22, MB12/22-RGD, ARC187, ARC1905, SSL7, and OmCI.

16. The method of any one of claims 1-15, wherein the step of determining whether the test compound binds to the variant C5 polypeptide or the wild-type polypeptide is performed by surface plasmon resonance, biolayer interferometry, or mass spectrometry.

17. The method of any one of claims 1-15, wherein the step of determining whether the test compound binds to the variant C5 polypeptide or the wild-type polypeptide is performed using an immunoassay.

18. The method of claim 17, wherein the immunoassay is an enzyme-linked immunosorbent assay (ELISA) or a radioimmunoassay (RIA).

19. The method of any one of claims 1-15, wherein the step of determining whether the test compound binds to the variant C5 polypeptide comprises determining the binding affinity of the test compound for the variant C5 polypeptide.

20. The method of any one of claims 1-19, wherein the step of determining whether the test compound binds to the wild-type C5 polypeptide comprises determining the binding affinity of the test compound for the wild-type C5 polypeptide.

21. The method of claim 19 or 20, wherein the binding affinity is determined by surface plasmon resonance, biolayer interferometry, or mass spectrometry.

22. The method of any one of claims 1-21, wherein the test compound is selected from the group consisting of an antibody, a small molecule, a polypeptide, a polypeptide analog, a peptidomimetic, and an aptamer.

23. The method of any one of claims 1-22, wherein the test compound is rationally designed to bind the wild-type C5 polypeptide.

24. The method of claim 23, wherein the test compound is rationally designed to bind a C5 convertase-binding site of C5.

25. The method of claim 23, wherein the test compound is rationally designed to bind an epitope of C5 set forth between residues 872 and 892 of SEQ ID NO:2.

26. The method of any one of claims 1-25, wherein the variant C5 polypeptide comprises the amino acid sequence depicted in SEQ ID NO:47.

27. The method of any one of claims 1-25, wherein the variant C5 polypeptide comprises the amino acid sequence depicted in SEQ ID NO:48.

28. The method of any one of claims 1-25, wherein the variant C5 polypeptide comprises at least five consecutive amino acids of SEQ ID NO:47, inclusive of histidine 885.

29. The method of claim 28, wherein the variant C5 polypeptide comprises at least 10 consecutive amino acids of SEQ ID NO:47.

30. The method of claim 28, wherein the variant C5 polypeptide comprises at least 50 consecutive amino acids of SEQ ID NO:47.

31. The method of any one of claims 1-25, wherein the variant C5 polypeptide: (a) comprises at least 20 amino acids, (b) is at least 80% identical to a corresponding at least 20 amino acid sequence of SEQ ID NO:47, and (c) comprises histidine 885 of SEQ ID NO:47.

32. The method of any one of claims 1-25, wherein the test compound is rationally designed to bind an epitope of C5 comprising at least five amino acids of SEQ ID NO:2 or 47, inclusive of amino acid 885.

33. The method of claim 32, wherein the epitope of C5 comprises at least 10 consecutive amino acids of SEQ ID NO:2 or 47.

34. The method of claim 33, wherein the epitope of C5 comprises at least 20 consecutive amino acids of SEQ ID NO:2 or 47.

35. The method of claim 33, wherein the epitope of C5 comprises at least 10 consecutive amino acids of SEQ ID NO:47, inclusive of histidine 885.

36. A method of identifying a compound that binds to a variant C5 polypeptide at a region within or overlapping with the region of the wild-type C5 polypeptide bound by a known wild-type C5 antagonist, the method comprising:
(i) providing a variant C5 polypeptide to which the known wild-type C5 antagonist compound: (a) does not bind or (b) binds with lower affinity as compared to the affinity of the known wild-type C5 antagonist for the wild-type C5 polypeptide;
(ii) determining whether a test compound binds to the variant C5 polypeptide; and
(iii) determining whether the test compound binds to the wild-type C5 polypeptide;
wherein a test compound that binds to the variant C5 polypeptide, but not to the wild-type C5 polypeptide or a test compound that preferentially binds to a variant C5 polypeptide as compared to the wild-type C5 polypeptide, is indicative of a compound that binds to the variant C5 polypeptide at a region within or overlapping with the region of the wild-type C5 polypeptide bound by the known wild-type C5 antagonist.

37. The method of claim 36, further comprising selecting a test compound that binds to the variant C5 polypeptide, but not to the wild-type C5 polypeptide or a test compound that preferentially binds to a variant C5 polypeptide as compared to the wild-type C5 polypeptide.

38. A method of identifying a compound that binds to a wild-type C5 polypeptide at a region within or overlapping with the region of the wild-type C5 polypeptide bound by a known wild-type C5 antagonist, the method comprising:
(i) providing a variant C5 polypeptide to which the known wild-type C5 antagonist compound: (a) does not bind or (b) binds with lower affinity as compared to the affinity of the known wild-type C5 antagonist for the wild-type C5 polypeptide;
(ii) determining the binding affinity of a test compound to the variant C5 polypeptide;
(iii) determining the binding affinity of the test compound to the wild-type C5 polypeptide; and
(iv) comparing the binding affinity from step (ii) to the binding affinity from step (iii),
wherein greater affinity of the test compound for the wild-type C5 polypeptide, as compared to the affinity of the test compound for the variant C5 polypeptide, is indicative of a compound that binds to the wild-type C5 polypeptide at a region within or overlapping with the region of the wild-type C5 polypeptide bound by the known wild-type C5 antagonist.

39. The method of claim 38, further comprising selecting a test compound having a greater affinity for the wild-type C5 polypeptide as compared to the affinity of the test compound for the variant C5 polypeptide.

40. A method of identifying a compound that binds to a variant C5 polypeptide at a region within or overlapping with the region of the wild-type polypeptide bound by a known wild-type antagonist, the method comprising:
(i) providing a variant C5 polypeptide to which the known wild-type C5 antagonist compound (a) does not bind or (b) binds with lower affinity as compared to the affinity of the known wild-type C5 antagonist for the wild-type C5 polypeptide;
(ii) determining the binding affinity of a test compound to the variant C5 polypeptide;
(iii) determining the binding affinity of the test compound to the wild-type C5 polypeptide; and
(iv) comparing the binding affinity from step (ii) to the binding affinity from step (iii),
wherein greater affinity of the test compound for the variant C5 polypeptide, as compared to the affinity of the test compound for the wild-type C5 polypeptide, is indicative of a compound that binds to the variant C5 polypeptide at a region within or overlapping with the region of the wild-type C5 polypeptide bound by the known wild-type C5 antagonist.

41. The method of claim 40, further comprising selecting a test compound having a greater affinity for the variant C5 polypeptide as compared to the affinity of the test compound for the wild-type C5 polypeptide.

42. The method of any one of claims 36-41, wherein the test compound inhibits cleavage of C5 into fragments C5a and C5b.

43. The method of any one of claims 36-42, further comprising determining whether the test compound inhibits the cleavage of C5 into fragments C5a and C5b.

44. The method of claim 43, wherein a hemolytic assay is used to determine whether the test compound inhibits the cleavage of C5 into fragments C5a and C5b.

45. The method of any one of claims 36-44, wherein the wild-type C5 polypeptide comprises an amino acid sequence set forth in SEQ ID NO:2 or a fragment thereof.

46. The method of any one of claims 36-45, wherein the variant C5 polypeptide comprises a deletion, an insertion, or a substitution, as compared to the wild-type C5 polypeptide.

47. The method of claim 46, wherein the deletion, insertion, or substitution is at a C5 convertase-binding site.

48. The method of claim 46, wherein the deletion, insertion, or substitution is present between residues 872 and 892 of SEQ ID NO:2.

49. The method of claim 46, wherein the deletion, insertion, or substitution is present at the epitope to which the known wild-type C5 antagonist binds.

50. The method of any one of claims 36-49, wherein the variant C5 polypeptide is present in subjects non-responsive to treatment with the known C5 antagonist.

51. The method of any one of claims 36-50, wherein the known wild-type C5 antagonist is an anti-C5 antibody or an antigen binding fragment thereof, a small molecule, a polypeptide, a polypeptide analog, a peptidomimetic, or an aptamer.

52. The method of claim 51, wherein the known wild-type C5 antagonist is eculizumab.

53. The method of claim 51, wherein the known wild-type C5 antagonist is pexelizumab.

54. The method of claim 51, wherein the known wild-type C5 antagonist is selected from the group consisting of MB12/22, MB12/22-RGD, ARC187, ARC1905, SSL7, and OmCI.

55. The method of any one of claims 36-54, wherein the step of determining whether the test compound binds to the variant C5 polypeptide or the wild-type polypeptide is performed by surface plasmon resonance, biolayer interferometry, or mass spectrometry.

56. The method of any one of claims 36-55, wherein the step of determining whether the test compound binds to the variant C5 polypeptide or the wild-type polypeptide is performed using an immunoassay.

57. The method of claim 56, wherein the immunoassay is an enzyme-linked immunosorbent assay (ELISA) or a radioimmunoassay (RIA).

58. The method of any one of claims 36-57, wherein the step of determining whether the test compound binds to the variant C5 polypeptide comprises determining the binding affinity of the test compound for the variant C5 polypeptide.

59. The method of any one of claims 36-58, wherein the step of determining whether the test compound binds to the wild-type C5 polypeptide comprises determining the binding affinity of the test compound for the wild-type C5 polypeptide.

60. The method of claim 58 or 59, wherein the binding affinity is determined by surface plasmon resonance, biolayer interferometry, or mass spectrometry.

61. The method of any one of claims 36-60, wherein the test compound is selected from the group consisting of an antibody, a small molecule, a polypeptide, a polypeptide analog, a peptidomimetic, and an aptamer.

62. The method of any one of claims 36-61, wherein the test compound is rationally designed to bind the wild-type C5 polypeptide.

63. The method of claim 62, wherein the test compound is rationally designed to bind a C5 convertase-binding site of C5.

64. The method of claim 62, wherein the test compound is rationally designed to bind an epitope of C5 set forth between residues 872 and 892 of SEQ ID NO:2.

65. The method of any one of claims 36-64, wherein the variant C5 polypeptide comprises the amino acid sequence depicted in SEQ ID NO:47.

66. The method of any one of claims 36-64, wherein the variant C5 polypeptide comprises the amino acid sequence depicted in SEQ ID NO:48.

67. The method of any one of claims 36-64, wherein the variant C5 polypeptide comprises at least five consecutive amino acids of SEQ ID NO:47, inclusive of histidine 885.

68. The method of claim 67, wherein the variant C5 polypeptide comprises at least 10 consecutive amino acids of SEQ ID NO:47.

69. The method of claim 67, wherein the variant C5 polypeptide comprises at least 50 consecutive amino acids of SEQ ID NO:47.

70. The method of any one of claims 36-64, wherein the variant C5 polypeptide: (a) comprises at least 20 amino acids, (b) is at least 80% identical to a corresponding at least 20 amino acid sequence of SEQ ID NO:47, and (c) comprises histidine 885 of SEQ ID NO:47.

71. The method of any one of claims 36-64, wherein the test compound is rationally designed to bind an epitope of C5 comprising at least five consecutive amino acids of SEQ ID NO:2 or 47, inclusive of amino acid 885.

72. The method of claim 71, wherein the epitope of C5 comprises at least 10 consecutive amino acids of SEQ ID NO:2 or 47.

73. The method of claim 71, wherein the epitope of C5 comprises at least 20 consecutive amino acids of SEQ ID NO:2 or 47.

74. The method of claim 71, wherein the epitope of C5 comprises at least 10 consecutive amino acids of SEQ ID NO:2, inclusive of arginine 885.

75. A method of screening for a compound that binds to a wild-type C5 polypeptide, the method comprising:
(i) providing a variant C5 polypeptide to which a known wild-type C5 antagonist compound: (a) does not bind or (b) binds with lower affinity as compared to the affinity of the known wild-type C5 antagonist for a wild-type C5 polypeptide;
(ii) providing a library of test compounds;
(iii) screening a plurality of the test compounds for binding to the wild-type C5 polypeptide to identify test compounds that bind to the wild-type C5 polypeptide;
(iv) screening one or more of the test compounds identified in (iii) for binding to the variant C5 polypeptide; and
(v) selecting at least one test compound that binds to the wild-type C5 polypeptide but does not bind to the variant C5 polypeptide or preferentially binds to the wild-type C5 polypeptide as compared to the binding of the test compound to the variant C5 polypeptide.

76. A method of screening for a compound that binds to a variant C5 polypeptide, the method comprising:
(i) providing a variant C5 polypeptide to which a known wild-type C5 antagonist compound: (a) does not bind or (b) binds with lower affinity as compared to the affinity of the known wild-type C5 antagonist for a wild-type C5 polypeptide;
(ii) providing a library of test compounds;
(iii) screening a plurality of the test compounds for binding to the variant C5 polypeptide to identify test compounds that bind to the variant C5 polypeptide;
(iv) screening one or more of the test compounds identified in (iii) for binding to the wild-type C5 polypeptide; and
(v) selecting at least one test compound that binds to the variant C5 polypeptide but does not bind to the wild-type C5 polypeptide or preferentially binds to the variant C5 polypeptide as compared to the binding of the test compound to the wild-type C5 polypeptide.

77. The method of claim 75 or 76, wherein the test compound inhibits cleavage of C5 into fragments C5a and C5b.

78. The method of claim 75 or 76, further comprising determining whether the test compound inhibits the cleavage of C5 into fragments C5a and C5b.

79. The method of claim 78, wherein a hemolytic assay is used to determine whether the test compound inhibits the cleavage of C5 into fragments C5a and C5b.

80. The method of claim 75 or 76, wherein the wild-type C5 polypeptide comprises an amino acid sequence set forth in SEQ ID NO:2 or a fragment thereof.

81. The method of claim 75 or 76, wherein the variant C5 polypeptide comprises a deletion, an insertion, or a substitution.

82. The method of claim 81, wherein the deletion, insertion, or substitution is at a C5 convertase-binding site.

83. The method of claim 81, wherein the deletion, insertion, or substitution is present between residues 872 and 892 of SEQ ID NO:2.

84. The method of claim 81, wherein the deletion, insertion, or substitution is present at the epitope to which the known wild-type C5 antagonist binds.

85. The method of claim 75 or 76, wherein the variant C5 polypeptide is present in subjects non-responsive to treatment with the known C5 antagonist.

86. The method of claim 75 or 76, wherein the known wild-type C5 antagonist is an anti-C5 antibody or an antigen binding fragment thereof, a small molecule, a polypeptide, a polypeptide analog, a peptidomimetic, or an aptamer.

87. The method of claim 86, wherein the known wild-type C5 antagonist is eculizumab.

88. The method of claim 86, wherein the known wild-type C5 antagonist is pexelizumab.

89. The method of claim 86, wherein the known wild-type C5 antagonist is selected from the group consisting of MB12/22, MB12/22-RGD, ARC187, ARC1905, SSL7, and OmCI.

90. The method of any one of claims 75-89, wherein the step of determining whether the test compound binds to the variant C5 polypeptide or the wild-type polypeptide is performed by surface plasmon resonance, biolayer interferometry, or mass spectrometry.

91. The method of any one of claims 75-90, wherein the step of determining whether the test compound binds to the variant C5 polypeptide or the wild-type polypeptide is performed using an immunoassay.

92. The method of claim 91, wherein the immunoassay is an enzyme-linked immunosorbent assay (ELISA) or a radioimmunoassay (RIA).

93. The method of any one of claims 75-90, wherein the step of determining whether the test compound binds to the variant C5 polypeptide comprises determining the binding affinity of the test compound for the variant C5 polypeptide.

94. The method of any one of claims 75-93, wherein the step of determining whether the test compound binds to the wild-type C5 polypeptide comprises determining the binding affinity of the test compound for the wild-type C5 polypeptide.

95. The method of claim 93 or 94, wherein the binding affinity is determined by surface plasmon resonance, biolayer interferometry, or mass spectrometry.

96. The method of any one of claims 75-95, wherein the test compound is selected from the group consisting of an antibody, a small molecule, a polypeptide, a polypeptide analog, a peptidomimetic, and an aptamer.

97. The method of any one of claims 75-96, wherein the test compound is rationally designed to bind the wild-type C5 polypeptide.

98. The method of claim 97, wherein the test compound is rationally designed to bind a C5 convertase-binding site of C5.

99. The method of claim 97, wherein the test compound is rationally designed to bind an epitope of C5 set forth between residues 872 and 892 of SEQ ID NO:2.

100. The method of any one of claims 75-99, wherein the variant C5 polypeptide comprises the amino acid sequence depicted in SEQ ID NO:47.

101. The method of any one of claims 75-99, wherein the variant C5 polypeptide comprises the amino acid sequence depicted in SEQ ID NO:48.

102. The method of any one of claims 75-99, wherein the variant C5 polypeptide comprises at least five consecutive amino acids of SEQ ID NO:47, inclusive of histidine 885.

103. The method of claim 102, wherein the variant C5 polypeptide comprises at least 10 consecutive amino acids of SEQ ID NO:47.

104. The method of claim 102, wherein the variant C5 polypeptide comprises at least 50 consecutive amino acids of SEQ ID NO:47.

105. The method of any one of claims 75-99, wherein the variant C5 polypeptide: (a) comprises at least 20 amino acids, (b) is at least 80% identical to a corresponding at least 20 amino acid sequence of SEQ ID NO:47, and (c) comprises histidine 885 of SEQ ID NO:47.

106. The method of any one of claims 75-99, wherein the test compound is rationally designed to bind an epitope of C5 comprising at least five consecutive amino acids of SEQ ID NO:2 or 47, inclusive of amino acid 885.

107. The method of claim 106, wherein the epitope of C5 comprises at least 10 consecutive amino acids of SEQ ID NO:2 or 47.

108. The method of claim 106, wherein the epitope of C5 comprises at least 20 consecutive amino acids of SEQ ID NO:2 or 47.

109. The method of claim 106, wherein the epitope of C5 comprises at least 10 consecutive amino acids of SEQ ID NO:2, inclusive of arginine 885.

110. A method of identifying a compound that binds to a wild-type polypeptide at a region within or overlapping with the region of the wild-type polypeptide bound by a known agonist or antagonist of the wild-type polypeptide, the method comprising:
(i) providing a wild-type polypeptide to which a known agonist or antagonist compound binds;
(ii) providing a variant form of the wild-type polypeptide (variant polypeptide) to which the agonist or antagonist: (a) does not bind or (b) binds with lower affinity as compared to the affinity of the known agonist or antagonist for the wild-type polypeptide;
(iii) determining whether a test compound binds to the variant polypeptide; and
(iv) determining whether the test compound binds to the wild-type polypeptide;
wherein a test compound that binds to the wild-type polypeptide, but not to the variant polypeptide, or a test compound that preferentially binds to the wild-type polypeptide as compared to the variant polypeptide is indicative of a compound that binds to the wild-type polypeptide at a region within or overlapping with the region of the wild-type polypeptide bound by the known agonist or antagonist.

111. A method of identifying a compound that binds to a variant polypeptide at a region within or overlapping with the region of the wild-type form of the polypeptide bound by a known agonist or antagonist of the wild-type polypeptide, the method comprising:
(i) providing a wild-type polypeptide to which a known agonist or antagonist compound binds;
(ii) providing a variant form of the wild-type polypeptide (variant polypeptide) to which the agonist or antagonist: (a) does not bind or (b) binds with lower affinity as compared to the affinity of the known agonist or antagonist for the wild-type polypeptide;
(iii) determining whether a test compound binds to the variant polypeptide; and
(iv) determining whether the test compound binds to the wild-type polypeptide;
wherein a test compound that binds to the variant polypeptide, but not to the wild-type polypeptide, or a test compound that preferentially binds to the variant polypeptide as compared to the wild-type polypeptide is indicative of a compound that binds to the variant polypeptide at a region within or overlapping with the region of the wild-type polypeptide bound by the known agonist or antagonist.

112. A method of screening for a compound that binds to a wild-type polypeptide at a region within or overlapping with the region of the wild-type polypeptide bound by a known agonist or antagonist of the wild-type polypeptide, the method comprising:
(i) providing a wild-type polypeptide to which a known agonist or antagonist compound binds;
(ii) providing a variant form of the wild-type polypeptide (variant polypeptide) to which the agonist or antagonist: (a) does not bind or (b) binds with lower affinity as compared to the affinity of the known agonist or antagonist for the wild-type polypeptide;
(iii) providing a library of test compounds;
(iv) screening a plurality of the test compounds for binding to the variant polypeptide;
(v) screening a plurality of the test compounds for binding to the wild-type polypeptide; and
(vi) selecting one or more test compounds that bind to the wild-type polypeptide, but not to the variant polypeptide or that preferentially bind to the wild-type polypeptide as compared to the variant polypeptide, wherein such compounds are indicative of compounds that bind to the wild-type polypeptide at a region within or overlapping with the region of the wild-type polypeptide bound by the known agonist or antagonist.

113. A method of screening for a compound that binds to a variant form of a wild-type polypeptide at a region within or overlapping with the region of the wild-type polypeptide bound by a known agonist or antagonist of the wild-type polypeptide, the method comprising:
(i) providing a wild-type polypeptide to which a known agonist or antagonist compound binds;
(ii) providing a variant form of the wild-type polypeptide (variant polypeptide) to which the agonist or antagonist: (a) does not bind or (b) binds with lower affinity as compared to the affinity of the known agonist or antagonist for the wild-type polypeptide;
(iii) providing a library of test compounds;
(iv) screening a plurality of the test compounds for binding to the variant polypeptide;
(v) screening a plurality of the test compounds for binding to the wild-type polypeptide; and
(vi) selecting one or more test compounds that bind to the variant polypeptide, but not to the wild-type polypeptide or that preferentially bind to the variant polypeptide as compared to the wild-type polypeptide, wherein such compounds are indicative of compounds that bind to the variant polypeptide at a region within or overlapping with the region of the wild-type polypeptide bound by the known agonist or antagonist.

114. The method of any one of claims 110-113, wherein the step of determining whether the test compound binds to the variant polypeptide comprises determining the binding affinity of the test compound for the variant polypeptide.

115. The method of any one of claims 110-114, wherein the step of determining whether the test compound binds to the wild-type polypeptide comprises determining the binding affinity of the test compound for the wild-type polypeptide.

116. The method of any one of claims 110-115, wherein the test compound is a small molecule.

117. The method of claim 116, wherein a library of small molecule test compounds is subjected to the method.

118. The method of any one of claims 110-117, wherein the wild-type polypeptide is a human polypeptide.

119. The method of any one of claims 110-118, wherein the known agonist or antagonist is a drug for treating human disease.

120. The method of any one of claims 110-119, wherein the known agonist or antagonist is an antibody or antigen-binding fragment thereof.

121. The method of any one of claims 110-120, wherein the wild-type polypeptide is a growth factor, a cytokine, or a chemokine.

122. The method of any one of claims 110-120, wherein the wild-type polypeptide is a growth factor receptor polypeptide or a growth factor-binding fragment thereof, a cytokine receptor polypeptide or a cytokine-binding fragment thereof, or a chemokine receptor polypeptide or a chemokine-binding fragment thereof.

123. The method of any one of claims 110-120, wherein the wild-type polypeptide is a component of the complement cascade.

124. The method of claim 123, wherein the component of the complement cascade is selected from the group consisting of C1, C1r, C1s, C1q, C2, C3, C3a, C3b, C4, C4a, C4b, C5, C5a, C5b, C6, C7, C8, C9, MASP1, MASP2, properdin, factor D, factor B, factor H, and factor I.

125. The method of claim 123 or 124, wherein the test compound inhibits cleavage of C5 into fragments C5a and C5b.

126. The method of claim 125, further comprising determining whether the test compound inhibits the cleavage of C5 into fragments C5a and C5b.

127. The method of claim 126, wherein a hemolytic assay is used to determine whether the test compound inhibits the cleavage of C5 into fragments C5a and C5b.

128. The method of any one of claims 123-127, wherein the wild-type polypeptide is a wild-type C5 polypeptide and wherein the wild-type C5 polypeptide comprises the amino acid sequence set forth in SEQ ID NO:2 or a fragment thereof.

129. The method of any one of claims 110-128, wherein the variant polypeptide comprises a deletion, an insertion, or a substitution relative to the wild-type polypeptide.

130. The method of any one of claims 110-129, wherein the variant polypeptide is a variant C5 polypeptide.

131. The method of claim 130, wherein the variant C5 polypeptide comprises a deletion, insertion, or substitution at a C5 convertase-binding site.

132. The method of claim 131, wherein the deletion, insertion, or substitution is present between residues 872 and 892 of SEQ ID NO:2.

133. The method of any one of claims 130-132, wherein the variant C5 polypeptide comprises the amino acid sequence depicted in SEQ ID NO:47.

134. The method of any one of claims 130-133, wherein the variant C5 polypeptide comprises the amino acid sequence depicted in SEQ ID NO:48.

135. The method of any one of claims 130-133, wherein the variant C5 polypeptide comprises at least five consecutive amino acids of SEQ ID NO:47, inclusive of histidine 885.

136. The method of claim 135, wherein the variant C5 polypeptide comprises at least 10 consecutive amino acids of SEQ ID NO:47.

137. The method of claim 135, wherein the variant C5 polypeptide comprises at least 50 consecutive amino acids of SEQ ID NO:47.

138. The method of any one of claims 130-133, wherein the variant C5 polypeptide: (a) comprises at least 20 amino acids, (b) is at least 80% identical to a corresponding at least 20 amino acid sequence of SEQ ID NO:47, and (c) comprises histidine 885 of SEQ ID NO:47.

139. The method of any one of claims 110-138, wherein the test compound is rationally designed to bind the wild-type polypeptide.

140. The method of claim 139, wherein the test compound is rationally designed to bind a wild-type C5 polypeptide.

141. The method of claim 140, wherein the test compound is rationally designed to bind a C5 convertase-binding site of C5.

142. The method of claim 140, wherein the test compound is rationally designed to bind an epitope of wild-type C5 comprising residues 872 and 892 of SEQ ID NO:2.

143. The method of claim 139, wherein the test compound is rationally designed to bind an epitope of C5 comprising at least five amino acids of SEQ ID NO:2 or 47, inclusive of amino acid 885.

144. The method of claim 143, wherein the epitope of C5 comprises at least 10 consecutive amino acids of SEQ ID NO:2 or 47.

145. The method of claim 143, wherein the epitope of C5 comprises at least 20 consecutive amino acids of SEQ ID NO:2 or 47.

146. The method of claim 143, wherein the epitope of C5 comprises at least 10 consecutive amino acids of SEQ ID NO:2, inclusive of arginine 885.

147. The method of any one of claims 110-146, wherein the deletion, insertion, or substitution is present within or adjacent to the epitope to which the known agonist or antagonist binds.

148. The method of any one of claims 110-146, wherein the variant polypeptide is present in a subject non-responsive to treatment with the known agonist or antagonist.

149. The method of any one of claim 110-120 or 123-147, wherein the known antagonist is eculizumab.

150. The method of any one of claim 110-120 or 123-147, wherein the known antagonist is pexelizumab.

151. The method of any one of claim 110-120 or 123-147, wherein the known antagonist is selected from the group consisting of: MB12/22, MB12/22-RGD, ARC187, ARC1905, SSL7, and OmCI.

152. A method for identifying a compound that binds to a wild-type polypeptide at a region within or overlapping with the region of a variant form of the wild-type polypeptide (variant polypeptide) bound by a known agonist or antagonist of the variant polypeptide, the method comprising:
(i) providing a variant polypeptide to which a known agonist or antagonist compound binds;
(ii) providing the wild-type polypeptide to which the known agonist or antagonist: (a) does not bind or (b) binds with lower affinity as compared to the affinity of the known agonist or antagonist for the variant polypeptide;
(iii) determining whether a test compound binds to the variant polypeptide; and
(iv) determining whether the test compound binds to the wild-type polypeptide;
wherein a test compound that binds to the wild-type polypeptide, but not to the variant polypeptide, or a test compound that preferentially binds to the wild-type polypeptide as compared to the variant polypeptide is indicative of a compound that binds to the wild-type polypeptide at a region within or overlapping with the region of the variant polypeptide bound by the known agonist or antagonist.

153. A method for identifying a compound that binds to a variant form of a wild-type polypeptide at a region within or overlapping with the region of the variant form of the polypeptide (variant polypeptide) bound by a known agonist or antagonist of the variant polypeptide, the method comprising:
(i) providing a variant polypeptide to which a known agonist or antagonist compound binds;
(ii) providing the wild-type polypeptide to which the known agonist or antagonist: (a) does not bind or (b) binds with lower affinity as compared to the affinity of the known agonist or antagonist for the variant polypeptide;
(iii) determining whether a test compound binds to the variant polypeptide; and
(iv) determining whether the test compound binds to the wild-type polypeptide;
wherein a test compound that binds to the variant polypeptide, but not to the wild-type polypeptide, or a test compound that preferentially binds to the variant polypeptide as compared to the wild-type polypeptide is indicative of a compound that binds to the variant polypeptide at a region within or overlapping with the region of the variant polypeptide bound by the known agonist or antagonist.

154. A method of screening for a compound that binds to a wild-type polypeptide at a region within or overlapping with the region of a variant form of the wild-type polypeptide (variant polypeptide) bound by a known agonist or antagonist of the variant polypeptide, the method comprising:
(i) providing the variant polypeptide to which a known agonist or antagonist compound binds;
(ii) providing the wild-type polypeptide to which the known agonist or antagonist: (a) does not bind or (b) binds with lower affinity as compared to the affinity of the known agonist or antagonist for the variant polypeptide;
(iii) providing a library of test compounds;
(iv) screening a plurality of the test compounds for binding to the variant polypeptide;
(v) screening a plurality of the test compounds for binding to the wild-type polypeptide; and
(vi) selecting one or more test compounds that bind to the wild-type polypeptide, but not to the variant polypeptide or that preferentially bind to the wild-type polypeptide as compared to the variant polypeptide, wherein such compounds are indicative of compounds that bind to the wild-type polypeptide at a region within or overlapping with the region of the variant polypeptide bound by the known agonist or antagonist.

155. A method for screening for a compound that binds to a variant form of a wild-type polypeptide (variant polypeptide) at a region within or overlapping with the region of the variant polypeptide bound by a known agonist or antagonist of the variant polypeptide, the method comprising:
(i) providing a variant polypeptide to which a known agonist or antagonist compound binds;
(ii) providing the wild-type polypeptide to which the known agonist or antagonist: (a) does not bind or (b) binds with lower affinity as compared to the affinity of the known agonist or antagonist for the variant polypeptide;
(iii) providing a library of test compounds;
(iv) screening a plurality of the test compounds for binding to the variant polypeptide;
(v) screening a plurality of the test compounds for binding to the wild-type polypeptide; and
(vi) selecting one or more test compounds that bind to the variant polypeptide, but not to the wild-type polypeptide or that preferentially bind to the variant polypeptide as compared to the wild-type polypeptide, wherein such compounds are indicative of compounds that bind to the variant polypeptide at a region within or overlapping with the region of the variant polypeptide bound by the known agonist or antagonist.
